(19) United States
(12) Patent Application Publication
(10) Pub. No.: US 2013/0011361 A1
(43) Pub. Date: Jan. 10, 2013

(54) PYRAZOLE DERIVATIVES WHICH MODULATE STEAROYL-COA DESATURASE

(75) Inventors: Natalie Dales, Arlington, MA (US); Jianmin Fu, Coquitlam (CA); Qi Jia, Burnaby (CA); Natalia Pokrovskaia, New Westminster (CA); Shaoyi Sun, Coquitlam (CA); Zaihui Zhang, Vancouver (CA)

(73) Assignees: XENON PHARMACEUTICALS INC., Burnaby, BC (CA); NOVARTIS AG, Basel (CH)

(21) Appl. No.: 13/391,900

(22) PCT Filed: Oct. 1, 2010
(86) PCT No.: PCT/EP10/64672

§ 371 Date: Feb. 23, 2012

Related U.S. Application Data
(60) Provisional application No. 61/247,634, filed on Oct. 1, 2009.

(51) Int. Cl. A61K 31/4439; A61K 31/4196; C07D 401/14; C07D 403/14; A61K 31/4178; C07D 413/14; A61K 31/422; C07D 417/14; A61K 31/427; A61K 31/455; A61K 38/28; A61K 38/26; A61P 3/10; A61P 9/10; A61P 3/00; A61P 9/08; A61P 7/02; A61P 3/04; A61P 3/06; A61P 9/12; A61P 19/06; A61P 17/00; A61P 17/10; A61P 17/06; A61P 17/02; A61K 31/785; C07D 403/04

(52) U.S. Cl. 424/78.08; 548/263.2; 514/384; 546/272.4; 514/341; 546/274.4; 548/312.4; 514/392; 548/247; 514/378; 548/204; 514/365; 548/236; 514/374; 514/356; 514/6.5; 514/7.2; 514/5.3; 514/7.4; 514/11.7

(57) ABSTRACT

The present invention provides heterocyclic derivatives of formula (I) that modulate the activity of stearoyl-CoA desaturase. Methods of using such derivatives to modulate the activity of stearoyl-CoA desaturase and pharmaceutical compositions comprising such derivatives are also encompassed.

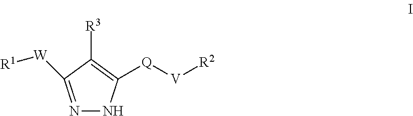

The present invention relates generally to the field of inhibitors of stearoyl-CoA desaturase, such as heterocyclic derivatives, and uses for such compounds in treating and/or preventing various human diseases, including those mediated by stearoyl-CoA desaturase (SCD) enzymes, preferably SCD1, especially diseases related to elevated lipid levels, cardiovascular disease, diabetes, obesity, metabolic syndrome, dermatological disorders and the like.

Acyl desaturase enzymes catalyze the formation of a double bond in fatty acids derived from either dietary sources or de novo synthesis in the liver. In mammals, at least three fatty acid desaturases exists, each with differing specificity: delta-9, delta-6, and delta-5, which introduce a double bond at the 9-10, 6-7, and 5-6 positions respectively.

Stearoyl-CoA desaturases (SCDs) act with cofactors (other agents) such as NADPH, cytochrome b5, cytochrome b5 reductase, Fe, and molecular O₂ to introduce a double bond into the C9-C10 position (delta 9) of saturated fatty acids, when conjugated to Coenzyme A (CoA). The preferred substrates are palmitoyl-CoA (16:0) and stearoyl-CoA (18:0), which are converted to palmitoleoyl-CoA (16:1) and oleyl-CoA (18:1), respectively. The resulting mono-unsaturated fatty acids are substrates for further metabolism by fatty acid elongases or incorporation into phospholipids, triglycerides, and cholesterol esters. A number of mammalian SCD genes have been cloned. For example, two genes have been identified in humans (hSCD1 and hSCD5) and four SCD genes have been isolated from mouse (SCD1, SCD2, SCD3, and SCD4). While the basic biochemical role of SCD has been known in rats and mice since the 1970s (Jeffcoat, R. et al., Eur. J. Biochem. (1979), Vol. 101, No. 2, pp. 439-445; de Antueno, R. et al., Lipids (1993), Vol. 28, No. 4, pp. 285-290), it has only recently been directly implicated in human disease processes.

The two human SCD genes have been previously described: hSCD1 by Brownlie et. al., PCT published patent application, WO 01/62954, and hSCD5 by Brownlie, PCT published patent application, WO 02/26944.

The present invention provides heterocyclic derivatives and pharmaceutical compositions containing such derivatives that are useful in modulating stearoyl-CoA desaturase (SCD) activity and regulating lipid levels, especially plasma lipid levels, and which are useful in the treatment of SCD-mediated diseases such as diseases related to dyslipidemia and disorders of lipid metabolism, especially diseases related to elevated lipid levels, cardiovascular disease, diabetes, obesity, metabolic syndrome, dermatological disorders and the like.

Accordingly, in one aspect, the invention provides a compound of Formula (I):

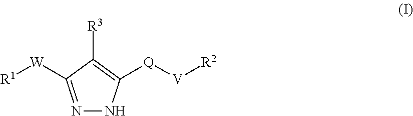

wherein,
Q is

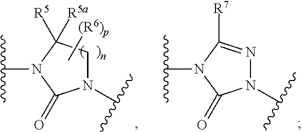

W is —N(R⁸)C(O)—, —C(O)N(R⁸)—, C₁-C₆alkylene, C₂-C₆alkeneylene, C₂-C₆alkynylene or a direct bond;
v is selected from a C₁-C₆alkylene;
   n is 1, 2, or 3;
   p is 0, 1, 2, 3, 4, 5, or 6;
   R¹ is hydrogen, an optionally substituted C₁-C₇alkyl, C₂-C₆alkenyl, C₂-C₆alkynyl, C₁-C₇alkoxy, hydroxyC₁-C₄alkyl, C₁-C₇alkoxyC₁-C₄alkyl, an optionally substituted C₃-C₇cycloalkyl, an optionally substituted C₃-C₇cycloalkylC₁-C₄alkyl, an optionally substituted C₆-C₁₀aryl, haloC₁-C₄alkyl, an optionally substituted C₆-C₁₀arylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
   R² is C₃-C₇alkyl, haloC₁-C₄alkyl, C₂-C₆alkenyl, C₂-C₆alkynyl, C₁-C₇alkoxy, hydroxy, hydroxyC₁-C₄alkyl, C₁-C₆alkoxyC₁-C₄alkyl, an optionally substituted C₃-C₇cycloalkyl, an optionally substituted C₆-C₁₀aryl, an optionally substituted C₂-C₁₀heterocyclyl, or and optionally substituted C₁-C₁₀heteroaryl, provided that V—R² is not quinolin-4-ylmethyl when R¹ is an alkyl;
   R³ is hydrogen, C₁-C₆alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₂-C₁₀heterocyclyl, C₆-C₁₀aryl, C₆-C₁₀arylC₁-C₄alkyl, C₁-C₁₀heteroaryl, halo, haloC₁-C₄alkyl, trifluoromethoxy, cyano, hydroxy, or —N(R⁸)₂;
   R⁵ and R^{5a} are independently selected from hydrogen, C₁-C₆alkyl, haloC₁-C₄alkyl, hydroxy, hydroxyC₁-C₄alkyl, C₁-C₆alkoxy, C₃-C₇cycloalkylC₁-C₄alkyl and C₆-C₁₀arylC₁-C₄alkyl;
   or R⁵ and R^{5a} are together to form an oxo (═O) group, or to form a C₃-C₇cycloalkyl;
   R⁶, for each occurrence, is independently selected from C₁-C₆alkyl, C₆-C₁₀aryl, C₃-C₇cycloalkyl, C₁-C₁₀heteroaryl, C₂-C₁₀heterocyclyl, hydroxyC₁-C₄alkyl, haloC₁-C₄alkyl, C₁-C₆alkoxy, C₃-C₇cycloalkylC₁-C₄alkyl, C₆-C₁₀arylC₁-C₄alkyl-N(R⁸)C(O)R¹², —C(O)N(R⁸)R¹², —OC(O)N(R⁸)R¹², —N(R⁸)C(O)OR¹², —N(R⁸)C(O)N(R⁸)R¹², —OR¹², —SR¹², —N(R⁸)R¹², —S(O)^{t}R¹², —N(R⁸)S(O)₂R¹², —S(O)₂N(R⁸)R¹², —OS(O)₂N(R⁸)R¹², —C(O)R¹², —OC(O)R¹², —N(R⁸)C(═N(R^{8a}))N(R⁸)R¹², —N(R⁸)C(═S)N(R⁸)R¹², —N(R⁸)((R^{8a})N═)CR¹², and —C(═N(R^{8a}))N(R⁸)R¹²;
   or R⁵ and R⁸ on adjacent carbons together to form a C₃-C₇cycloalkyl or C₆-C₁₀aryl;
   R⁷ is hydrogen, C₁-C₇alkyl, haloC₁-C₄alkyl, C₉-C₁₀aryl, C₃-C₇cycloalkyl, C₁-C₁₀heteroaryl, C₂-C₁₀heterocyclyl, hydroxyC₁-C₄alkyl, C₃-C₇cycloalkylC₁-C₄alkyl or aralkyl;
   R⁸, for each occurrence, is independently selected from hydrogen, C₁-C₇alkyl, hydroxyC₁-C₄alkyl, C₃-C₇cycloalkyl, C₃-C₇cycloalkylC₁-C₄alkyl, C₆-C₁₀aryl, C₁-C₁₀heteroaryl, C₂-C₁₀heterocyclyl and aralkyl; and
   R¹, for each occurrence, is independently selected from hydrogen, C₁-C₇alkyl, C₃-C₇cycloalkyl, C₃-C₇cycloalkylC₁-C₄alkyl, and cyano;
   R¹², for each occurrence, is independently selected from hydrogen, C₃-C₇alkyl, C₂-C₆alkenyl, C₂-C₆alkynyl, C₁-C₇alkoxy, hydroxy, hydroxyC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, aralkyl, aralkyloxy, C₂-C₁₀heterocyclyl, C₂-C₁₀heterocyclylC₁-C₄alkyl, C₁-C₁₀heteroaryl, and C₁-C₁₀heteroarylC₁-C₄alkyl;
      or a pharmaceutically acceptable salt thereof.

In another aspect, the invention provides methods of treating an SCD-mediated disease or condition in a mammal, preferably a human, wherein the methods comprise administering to the mammal in need thereof a therapeutically effective amount of a compound of the invention as set forth above.

In another aspect, the invention provides compounds or pharmaceutical compositions useful in treating, preventing and/or diagnosing a disease or condition relating to SCD biological activity such as the diseases encompassed by cardiovascular disorders and/or metabolic syndrome (including dyslipidemia, insulin resistance and obesity).

In another aspect, the invention provides compounds or pharmaceutical compositions useful in treating, preventing and/or diagnosing a disease or condition relating to SCD biological activity such as the diseases encompassed by dermatological disorders including acne.

In another aspect, the invention provides methods of preventing or treating a disease or condition related to elevated lipid levels, such as plasma lipid levels, especially elevated triglyceride or cholesterol levels, in a patient afflicted with such elevated levels, comprising administering to said patient a therapeutically or prophylactically effective amount of a compound or composition as disclosed herein. The present invention also relates to novel compounds having therapeutic ability to reduce lipid levels in an animal, especially triglyceride and cholesterol levels.

In another aspect, the invention provides pharmaceutical compositions comprising a compound of the invention as set forth above, or a pharmaceutically acceptable salt thereof, and a pharmaceutically acceptable excipient. In one embodiment, the present invention relates to a pharmaceutical composition comprising a compound of the invention, or a pharmaceutically acceptable salt thereof, in a pharmaceutically acceptable carrier, wherein the compound is present in an amount effective to modulate triglyceride level or to treat diseases related to dyslipidemia and disorders of lipid metabolism when administered to an animal, preferably a mammal, most preferably a human patient. In one embodiment, an animal, such as a human, has an elevated lipid level, such as elevated plasma triglycerides or cholesterol, before administration of said composition and said compound is present in an amount effective to reduce said lipid level.

In another aspect, the invention provides methods for treating a patient for, or protecting a patient from developing, a disease or condition mediated by stearoyl-CoA desaturase (SCD), which methods comprise administering to a patient afflicted with such disease or condition, or at risk of developing such disease or condition, a therapeutically effective amount of a compound, or a pharmaceutically acceptable salt thereof, that inhibits activity of SCD in a patient when administered thereto.

In another aspect, the invention provides methods for treating a range of diseases involving lipid metabolism and/or lipid homeostasis utilizing compounds identified by the methods disclosed herein. In accordance therewith, there is disclosed herein a range of compounds having said activity, based on a screening assay for identifying, from a library of test compounds, a therapeutic agent which modulates the biological activity of said SCD and is useful in treating a human disorder or condition relating to serum levels of lipids, such as triglycerides, VLDL, HDL, LDL, and/or total cholesterol.

### Definitions

Certain chemical groups named herein are preceded by a shorthand notation indicating the total number of carbon atoms that are to be found in the indicated chemical group. For example, C₇-C₁₂alkyl describes an alkyl group, as defined below, having a total of 7 to 12 carbon atoms; C₄-C₁₂cycloalkyl describes a cycloalkyl group, as defined below, having a total of 4 to 12 carbon atoms; and a C6-C10arylC1-C4alkyl describes an arylalkyl group, as defined below, wherein the aryl group has a total of 6 to. The total number of carbons in the shorthand notation does not include carbons that may exist in substituents of the group described.

Accordingly, as used in the specification and appended claims, unless specified to the contrary, the following terms have the meaning indicated:
“Cyano” refers to the —CN radical;
“Hydroxy” refers to the —OH radical;
“Nitro” refers to the —NO₂ radical;
“Amino” refers to the —N(R¹⁴)₂ radical;
“Mercapto” refers to the —SR¹⁴ radical;
“Carboxy” refers to the —COOH radical;
“Trifluoromethyl” refers to the —CF₃ radical;
“Trifluoromethoxy” refers to the —OCF₃ radical;

“Alkyl” refers to a straight or branched hydrocarbon chain radical consisting solely of carbon and hydrogen atoms, containing no unsaturation, having from one to twelve carbon atoms, preferably one to eight carbon atoms, one to seven carbon atoms, one to six carbon atoms or one to four carbon atoms, and which is attached to the rest of the molecule by a single bond. Examples of alkyl groups include methyl, ethyl, n-propyl, 1-methylethyl (iso-propyl), n-butyl, n-pentyl, 1,1-dimethylethyl (t-butyl), and the like. An alkyl group may be optionally substituted by one or more of the following groups: alkyl, alkenyl, halo, haloalkyl, cyano, aryl, cycloalkyl, heterocyclyl, heteroaryl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁴, —N(R¹⁴)(S(O)ₜR¹⁶), —SR¹⁶, —S(O)ₜR¹⁶, and —S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl or heteroarylalkyl; and each R¹⁶ is alkyl, cycloalkyl, cycloalkylalkyl, aryl, aralkyl (e.g. tolyl), heterocyclyl, heterocyclylalkyl, heteroaryl or heteroarylalkyl; and each t is 1 to 2.

“Alkenyl” refers to a straight or branched hydrocarbon chain radical group consisting solely of carbon and hydrogen atoms, containing at least one double bond, having from two to twelve carbon atoms, preferably two to eight carbon atoms or two to six carbon atoms and which is attached to the rest of the molecule by a single bond. Examples of alkenyl groups include ethenyl, prop-1-enyl, but-1-enyl, pent-1-enyl, penta-1,4-dienyl, and the like. An alkenyl group may be optionally substituted by one or more of the following groups: alkyl, alkenyl, halo, haloalkyl, aryl, aralkyl, cycloalkyl, cycloalkylalkyl, heterocyclyl, heterocyclylalkyl, heteroaryl, heteroarylalkyl, —OR¹⁴, —OC(O)—R¹⁴N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶), —SR¹⁶, —S(O)ₜR¹⁶, and —S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; and each R¹⁶ is alkyl, cycloalkyl, cycloalkylalkyl, aryl, aralkyl, heterocyclyl, heterocyclylalkyl, heteroaryl or heteroarylalkyl; and where each t is 1 to 2.

“Alkynyl” refers to a straight or branched hydrocarbon chain radical group consisting solely of carbon and hydrogen atoms, containing at least one triple bond, having from two to twelve carbon atoms, preferably two to eight carbon atoms or two to six carbon atoms and which is attached to the rest of the molecule by a single bond. An alkynyl group may be optionally substituted by one or more of the following groups: alkyl, alkenyl, halo, haloalkyl, aryl, aralkyl, cycloalkyl, cycloalkylalkyl, heterocyclyl, heterocyclylalkyl, heteroaryl, heteroarylalkyl, —OR¹⁴, —OC(O)—R¹⁴N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶), —SR¹⁶, —S(O)ₜR¹⁶, and —S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalky; and each R¹⁶ is alkyl, cycloalkyl, cycloalkylalkyl, aryl, aralkyl, heterocyclyl, heterocyclylalkyl, heteroaryl or heteroarylalkyl; and where each t is 1 to 2.

“Alkylene” refers to a straight or branched divalent saturated hydrocarbon chain consisting solely of carbon and hydrogen atoms, having from one to twelve carbon atoms, preferably from 1 to 6 carbon atoms, more preferable from 1 to 4 carbon atoms and linking the rest of the molecule to a radical group. Examples of alkylene groups include methylene, ethylene, propylene, n-butylene, and the like. The alkylene is attached to the rest of the molecule through a single bond and to the radical group through a single bond. The points of attachment of the alkeylene to the rest of the molecule and to the radical group can be through one carbon or any two carbons within the chain. An alkylene group may be optionally substituted by one or more of the following groups: alkyl, alkenyl, halo, haloalkyl, aryl, aralkyl, cycloalkyl, cycloalkylalkyl, heterocyclyl, heterocyclylalkyl, heteroaryl, heteroarylalkyl, —OR¹⁴, —OC(O)—R¹⁴, —N(R¹⁴)₂, —C(O)R¹⁴, —C(O)OR¹⁴, —C(O)N(R¹⁴)₂, —N(R¹⁴)C(O)OR¹⁶, —N(R¹⁴)C(O)R¹⁶, —N(R¹⁴)(S(O)ₜR¹⁶), —SR¹⁶, —S(O)ₜR¹⁶, and —S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; and each R¹⁶ is alkyl, haloalkyl, cycloalkyl, cycloalkylalkyl, aryl, aralkyl, heterocyclyl, heterocyclylalkyl, heteroaryl or heteroarylalkyl; and where each t is 1 to 2.

“Alkoxy” refers to a radical of the formula —ORₐ where Rₐ is an alkyl radical as generally defined above. The alkyl part of the alkoxy radical may be optionally substituted as defined above for an alkyl radical.

“Alkoxyalkyl” refers to a radical of the formula —R_{b}—O—Rₐ where Rₐ is an alkyl radical as defined above and R_{b} is an alkylene radical as defined above. The oxygen atom may be bonded to any carbon in the alkyl and alkylene radical. The alkyl and alkylene part of the alkoxyalkyl radical may be optionally substituted as defined above for an alkyl and an alkylene, respectively.

“Aryl” refers to aromatic monocyclic or multicyclic hydrocarbon ring system consisting only of hydrogen and carbon and containing from six to nineteen carbon atoms, preferably six to ten carbon atoms, where the ring system is aromatic. Aryl groups include, but are not limited to groups such as fluorenyl, phenyl and naphthyl. An aryl may be optionally substituted by one or more substituents selected from the group consisting of alkyl, alkenyl, alkynyl, halo, haloalkyl, cyano, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)R¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl; and where each t is 1 to 2.

“Arylalkyl” refers to a radical of the formula —R_{b}R_{c} where R_{b} is an alkylene radical as defined above and R_{c} is an aryl radical as defined above. Examples of arylalkyl groups include benzyl, phenylethyl, 2-naphthylprop-1-yl and the like. The aryl part of the arylalkyl radical may be optionally substituted as described above for an aryl group. The alkyl part of the arylalkyl radical may be optionally substituted as defined above for an alkyl group.

“Aryloxy” refers to a radical of the formula —OR_{c} where R_{c} is an aryl group as defined above. The aryl part of the aryloxy radical may be optionally substituted as defined above for an aryl group.

“Arylalkyloxy” refers to a radical of the formula —OR_{d} where R_{d} is an arylalkyl group as defined above. The arylalkyl part of the arylalkyloxy radical may be optionally substituted as defined above for an arylalkyl.

“Cycloalkyl” refers to a stable non-aromatic monocyclic or bicyclic hydrocarbon radical consisting solely of carbon and hydrogen atoms, having from three to fifteen carbon atoms, preferably having from three to twelve carbon atoms or from three to seven atoms, and which is saturated or unsaturated, but not aromatic, and is attached to the rest of the molecule by a single bond. Examples of cycloalkyls include cyclopropyl, cyclobutyl, cyclopentyl, cyclohexyl, decalinyl and the like. Cycloalkyl radicals may be optionally substituted by one or more substituents selected from the group consisting of alkyl, alkenyl, alkynyl, halo, haloalkyl, cyano, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁶—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl; and where each t is 1 to 2.

“Cycloalkylalkyl” refers to a radical of the formula —R_{b}R_{d} where R_{b} is an alkylene radical as defined above and Rₑ is a cycloalkyl radical as defined above. The cycloalkyl part of the cycloalkylalkyl radical may be optionally substituted as defined above for a cycloalkyl radical. The alkylene part of the cycloalkylalkyl radical may be optionally substituted as defined above for an alkylene radical.

“Halo” refers to bromo, chloro, fluoro or iodo.

“Haloalkyl” refers to an alkyl radical, as defined above, that is substituted by one or more halo radicals, as defined above. Examples of haloalkyl groups include trifluoromethyl, difluoromethyl, trichloromethyl, 2,2,2-trifluoroethyl, 1-fluoromethyl-2-fluoroethyl, 3-bromo-2-fluoropropyl, 1-bromomethyl-2-bromoethyl, and the like. The alkyl part of the haloalkyl radical may optionally be further substituted as defined above for an alkyl group.

“Heterocyclyl” refers to a stable 3- to 18-membered, non-aromatic ring radical which consists of carbon atoms and from one to five heteroatoms selected from the group consisting of nitrogen, oxygen and sulfur, preferably having from two to ten carbon atoms. The heterocyclyl radical may be a monocyclic, bicyclic or tricyclic ring system, which may include fused or bridged ring systems, wherein the fused or bridged rings may be saturated, partially unsaturated, or aromatic. For the purposes of this invention, a ring system containing heteroatoms is considered to be a heterocycyl if the point of attachment to another moiety is on a non-aromatic ring. Nitrogen or sulfur atoms in the heterocyclyl radical may be optionally oxidized; the nitrogen atom may be optionally alkylated/substituted. Examples of such heterocyclyl radicals include, but are not limited to, dioxolanyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl, homopiperidinyl, homopiperazinyl, and quinuclidinyl. Heterocyclyl radicals may be optionally substituted by one or more substituents selected from the group consisting of alkyl, alkenyl, halo, haloalkyl, cyano, oxo, thioxo, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), R¹⁵—SR¹⁶, —R¹⁵S(O)ₜR¹⁶, and —R¹⁵—S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl; and where each t is 1 to 2; and where each of the above substituents is unsubstituted.

“Heterocyclylalkyl” refers to a radical of the formula —R_{b}R_{f} where R_{b} is an alkylene radical as defined above and R_{f} is a heterocyclyl radical as defined above, and if the heterocyclyl is a nitrogen-containing heterocyclyl, the heterocyclyl may be attached to the alkylene radical at the nitrogen atom or at a carbon atom. The alkylene part of the heterocyclylalkyl radical may be optionally substituted as defined above for an alkylene group. The heterocyclyl part of the heterocyclylalkyl radical may be optionally substituted as defined above for a heterocyclyl group.

“Heteroaryl” refers to a 5- to 18-membered aromatic ring radical which consists of carbon atoms and from one to five heteroatoms selected from the group consisting of nitrogen, oxygen and sulfur, preferably having from one to ten carbon atoms. A heteroaryl radical may be a monocyclic, bicyclic or tricyclic ring system, which may include fused or bridged ring systems, wherein the fused or bridged ring system may be saturated, partially saturated or aromatic. For the purposes of this invention, a ring system that includes heteroatoms is a heteroaryl if the point of attachment to another moeity is an aromatic ring. Nitrogen or sulfur atoms in the heteroaryl radical may be optionally oxidized; the nitrogen atom may be optionally alkylated/substituted. Examples include, but are not limited to, azepinyl, acridinyl, benzimidazolyl, benzthiazolyl, benzindolyl, benzothiadiazolyl, benzonaphthofuranyl, benzoxazolyl, benzodioxolyl, benzodioxinyl, benzopyranyl, benzopyranonyl, benzofuranyl, benzofuranonyl, benzothienyl, benzo[b]thiophenyl, benzothiophenyl, benzotriazolyl, benzo[4,6]imidazo[1,2-a]pyridinyl, benzo[c][1,2,5]oxadiazolyl, benzo[c][1,2,5]thiadiazolyl, carbazolyl, cinnolinyl, dibenzofuranyl, furanyl, furanonyl, isoquinolinyl, isothiazolyl, imidazolyl, indolyl, indazolyl, isoindolyl, indolinyl, isoindolinyl, indolizinyl, isoxazolyl, naphthyridinyl, oxadiazolyl, 2-oxoazepinyl, oxazolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, isoquinolinyl, thiazolyl, thiadiazolyl, triazolyl, tetrazolyl, triazinyl, and thiophenyl. A heteroaryl may be optionally substituted by one or more substituents selected from the group consisting of alkyl, alkenyl, alkynyl, halo, haloalkyl, cyano, oxo, thioxo, nitro, aryl, aralkyl, cycloalkyl, cycloalkylalkyl, heterocyclyl, heterocyclylalkyl, heteroaryl, heteroarylalkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), R¹⁵—SR¹⁶, —R¹⁵—S(O)ₜR¹⁶, and —R¹⁵—S(O)ₜN(R¹⁴)₂, where each R¹⁴ is independently hydrogen, alkyl, haloalkyl, cycloalkyl, cycloalkylalkyl, aryl, aralkyl, heterocyclyl, heterocyclylalkyl, heteroaryl, or heteroarylalkyl; each R¹⁵ is independently a direct bond or a straight or branched alkylene or alkenylene chain; and each R¹⁶ is alkyl, haloalkyl, cycloalkyl, cycloalkylalkyl, aryl, aralkyl, heterocyclyl, heterocyclylalkyl, heteroaryl or heteroarylalkyl and where t is 1 to 2.

“Heteroarylalkyl” refers to a radical of the formula —R_{b}R_{f} where R_{b} is an alkylene as defined above and R_{g} is a heteroaryl radical as defined above. The heteroaryl part of the heteroarylalkyl radical may be optionally substituted as defined above for a heteroaryl group. The alkylene part of the heteroarylalkyl radical may be optionally substituted as defined above for an alkylene group.

“Hydroxyalkyl” refers to an alkyl radical as defined above in which one or more (preferably one, two or three) hydrogen atoms have been replaced with a hydroxy group. The hydroxy group may be attached to the alkyl radical on any carbon within the alkyl radical. A hydroxyalkyl group may be optionally further substituted as defined above for an alkyl group.

“A multi-ring structure” refers to a multicyclic ring system comprised of two to four rings wherein the rings are independently selected from cycloalkyl, aryl, heterocyclyl or heteroaryl as defined above. Each cycloalkyl may be optionally substituted as defined above for a cycloalkyl group. Each aryl may be optionally substituted as defined above for an aryl group. Each heterocyclyl may be optionally substituted as defined above for a heterocyclyl group. Each heteroaryl may be optionally substituted as defined above for a heteroaryl group. The rings may be attached to each other through direct bonds or some or all of the rings may be fused to each other.

“Prodrugs” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof, but is converted in vivo to an active compound of the invention. Prodrugs are typically rapidly transformed in vivo to yield the parent compound of the invention, for example, by hydrolysis in blood or conversion in the gut or liver. The prodrug compound often offers advantages of solubility, tissue compatibility or delayed release in a mammalian organism (see, Bundgard, H., Design of Prodrugs (1985), pp. 7-9, 21-24 (Elsevier, Amsterdam)).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, Anglican Pharmaceutical Association arid Pergamon Press, 1987.

The term “prodrug” is also meant to include any covalently bonded carriers which release the active compound of the invention in vivo when such prodrug is administered to a mammalian subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention wherein a hydroxy, amino or mercapto or acid group is bonded to any group that, when the prodrug of the compound of the invention is administered to a mammalian subject, cleaves to form a free hydroxy, free amino or free mercapto or acid group, respectively. Examples of prodrugs include, but are not limited to, acetate, formate and benzoate derivatives of alcohol or amides of amine functional groups in the compounds of the invention and the like.

“Stable compound” and “stable structure” are meant to indicate a compound that is sufficiently robust to survive isolation to a useful degree of purity from a reaction mixture, and formulation into an efficacious therapeutic agent. A skilled artisan will recognize unstable combinations of substituents.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both pharmaceutically acceptable acid addition salts and pharmaceutically acceptable base addition salts.

“Pharmaceutically acceptable acid addition salt” refers to those salts which retain the biological effectiveness and properties of the free bases, which do not have undesirable biological activity or other undesirable activity, and which are formed with inorganic acids such as, but not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphorirc acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which do not have undesirable biological activity or other undesirable activity. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Often crystallizations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

A “pharmaceutical composition” refers to a formulation of a compound of the invention and a medium generally accepted in the art for the delivery of the biologically active compound to mammals, e.g., humans. Such a medium includes all pharmaceutically acceptable carriers, diluents or excipients thereof.

“Therapeutically effective amount” refers to that amount of a compound of the invention which, when administered to a mammal, preferably a human, is sufficient to effect treatment, as defined below, of an SCD-mediated disease or condition in the mammal, preferably a human. The amount of a compound of the invention which constitutes a “therapeutically effective amount” will vary depending on the compound, the condition and its severity, and the age and body weight of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or disorder of interest, and includes: (i) preventing the disease or condition from occurring in a mammal, in particular, when such mammal is predisposed to the condition but has not yet been diagnosed as having it; (ii) inhibiting the disease or condition, e.g., arresting its development; (iii) relieving the disease or condition, e.g., causing regression of the disease or condition; or (iv) reducing the risk of developing the disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

The compounds of the invention, or their pharmaceutically acceptable salts may contain one or more asymmetric centers and may thus give rise to enantiomers, diastereomers, and other stereoisomeric forms that may be defined, in terms of absolute stereochemistry, as (R)− or (S)− or, as (D)− or (L)− for amino acids. The present invention is meant to include all such possible isomers, as well as their racemic and optically pure forms. Optically active (+) and (−), (R)− and (S)−, or (D)− and (L)− isomers may be prepared using chiral synthons or chiral reagents, or resolved using conventional techniques, such as HPLC using a chiral column. When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

The present invention includes all pharmaceutically acceptable isotopically-labeled compounds of the invention wherein one or more atoms are replaced by atoms having the same atomic number, but an atomic mass or mass number different from the atomic mass or mass number usually found in nature.

Examples of isotopes suitable for inclusion in the compounds of the invention comprises isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³¹P and ³²P, and sulphur, such as ³⁵S.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Isotopically-labeled compounds of the invention can generally be prepared by conventional techniques known to those skilled in the art or by processes analogous to those described in the accompanying Examples and Preparations Sections using an appropriate isotopically-labeled reagent in place of the non-labeled reagent previously employed.

The chemical naming protocol and structure diagrams used herein employ and rely on the chemical naming features as utilized by Chemdraw versions 10.0 or 11.0 (available from Cambridgesoft Corp., Cambridge, Mass.) or ISIS draw version 2.5 (available from MDL information systems).

### EMBODIMENTS OF THE INVENTION

Throughout this specification and in the claims that follow, unless the context requires otherwise, the word “comprise”, or variations such as “comprises”, “comprised of”, “comprising” or “comprising of”, will be understood to imply the inclusion of a stated integer or step or group of integers or steps but not the exclusion of any other integer or step or group of integers or steps and therefore inclusive and open-ended in that additional elements.

Various embodiments of the invention are described below. It will be appreciated that the features specified in each embodiment may be combined with other specified features, to provide further embodiments.

One embodiment of the invention is a compound of Formula (I):

Accordingly, in one aspect, the invention provides a compound of Formula (I):

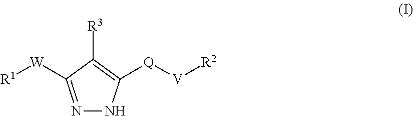

wherein,
Q is

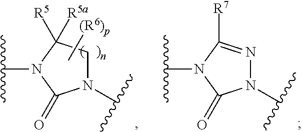

W is —N(R⁸)C(O)—, —C(O)N(R⁸)—, C₁-C₈alkylene, C₂-C₆alkeneylene, C₂-C₆alkynylene or a direct bond;
V is selected from a C₁-C₆alkylene;
   n is 1, 2, or 3;
   p is 0, 1, 2, 3, 4, 5, or 6;
   R¹ is hydrogen, an optionally substituted C₁-C₇alkyl, C₂-C₆alkenyl, C₂-C₆alkynyl, C₁-C₇alkoxy, hydroxyC₁-C₄alkyl, C₁-C₇alkoxyC₁-C₄alkyl, an optionally substituted C₃-C₇cycloalkyl, an optionally substituted C₃-C₇cycloalkylC₁-C₄alkyl, an optionally substituted C₆-C₁₀aryl, haloC₁-C₄alkyl, an optionally substituted C₆-C₁₀arylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
   R² is C₃-C₇alkyl, haloC₁-C₄alkyl, C₂-C₆alkenyl, C₂-C₆alkynyl, C₁-C₇alkoxy, hydroxy, hydroxyC₁-C₄alkyl, C₁-C₆alkoxyC₁-C₄alkyl, an optionally substituted C₃-C₇cycloalkyl, an optionally substituted C₆-C₁₀aryl, an optionally substituted C₂-C₁₀heterocyclyl, or and optionally substituted C₁-C₁₀heteroaryl, provided that V—R² is not quinolin-4-ylmethyl when R¹ is an alkyl;
   R³ is C₁-C₆alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₂-C₁₀heterocyclyl, C₆-C₁₀aryl, C₆-C₁₀arylC₁-C₄alkyl, C₁-C₁₀heteroaryl, halo, haloC₁-C₄alkyl, trifluoromethoxy, cyano, hydroxy, or —N(R⁸)₂;
   R⁵ and R^{5a} are independently selected from hydrogen, C₁-C₆alkyl, haloC₁-C₄alkyl, hydroxy, hydroxyC₁-C₄alkyl, C₁-C₆alkoxy, C₃-C₇cycloalkylC₁-C₄alkyl and C₆-C₁₀arylC₁-C₄alkyl;
   or R⁵ and R^{5a} are together to form an oxo (═O) group, or to form a C₃-C₇cycloalkyl;
   R⁶, for each occurrence, is independently selected from C₁-C₆alkyl, C₆-C₁₀aryl, C₃-C₇cycloalkyl, C₁-C₁₀heteroaryl, C₂-C₁₀heterocyclyl, hydroxyC₁-C₄alkyl, haloC₁-C₄alkyl, C₁-C₆alkoxy, C₃-C₇cycloalkylC₁-C₄alkyl, C₆-C₁₀arylC₁-C₄alkyl-N(R⁸)C(O)R¹², —C(O)N(R⁸)R¹², —OC(O)N(R⁸)R¹², —N(R⁸)C(O)OR¹², —N(R⁶)C(O)N(R⁸)R¹², —OR¹², —SR¹², —N(R⁸)R¹², —S(O)ₜR¹², —N(R⁸)S(O)₂R¹², —S(O)₂N(R⁸)R¹², —OS(O)₂N(R⁸)R¹², —C(O)R¹², —OC(O)R¹², —N(R⁸)C(═N(R^{8a}))N(R⁸)R¹², —N(R⁸)C(═S)N(R⁸)R¹², —N(R⁸)((R^{8a})N═)CR¹², and —C(═N(R^{8a})N(R⁸)R¹²;
   or R⁵ and R⁶ on adjacent carbons together to form a C₃-C₇cycloalkyl or C₆-C₁₀aryl;
   R⁷ is hydrogen, C₁-C₇alkyl, haloC₁-C₄alkyl, C₆-C₁₀aryl, C₃-C₇cycloalkyl, C₁-C₁₀heteroaryl, C₂-C₁₀heterocyclyl, hydroxyC₁-C₄alkyl, C₃-C₇cycloalkylC₁-C₄alkyl or aralkyl;
   R⁸, for each occurrence, is independently selected from hydrogen, C₁-C₇alkyl, hydroxyC₁-C₄alkyl, C₃-C₇cycloalkyl, C₃-C₇cycloalkylC₁-C₄alkyl, C₆-C₁₀aryl, C₁-C₁₀heteroaryl, C₂-C₁₀heterocyclyl and aralkyl; and
   R^{8a}, for each occurrence, is independently selected from hydrogen, C₁-C₇alkyl, C₃-C₇cycloalkyl, C₃-C₇cycloalkylC₁-C₄alkyl, and cyano;
   R¹², for each occurrence, is independently selected from hydrogen, C₃-C₇alkyl, C₂-C₆alkenyl, C₂-C₆alkynyl, C₁-C₇alkoxy, hydroxy, hydroxyC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, aralkyl, aralkyloxy, C₂-C₁₀heterocyclyl, C₂-C₁₀heterocyclylC₁-C₄alkyl, C₁-C₁₀heteroaryl, and C₁-C₁₀heteroarylC₁-C₄alkyl;
      or a pharmaceutically acceptable salt thereof.

In one embodiment, the invention provides compounds of formula (I) wherein Q is

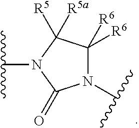

In another embodiment, the invention provides compounds of formula (I), wherein Q is

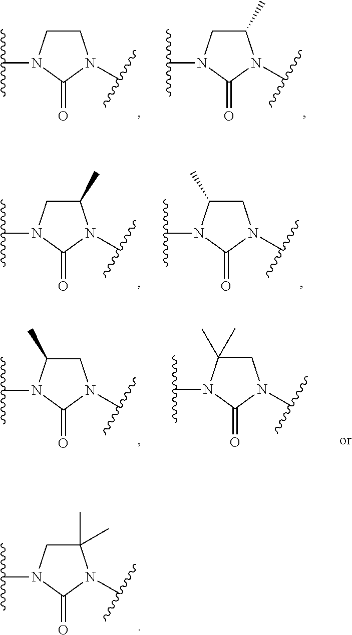

In another embodiment, the invention provides compounds of formula (I), wherein Q is

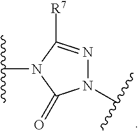

In another embodiment, the invention provides compounds of formula (I), wherein Q is

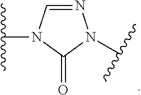 [SMILES: *n1cnn(*)c1=O]

In another embodiment, the invention provides compounds of formula (I), wherein W is —N(R⁸)C(O)—, and R¹ is hydrogen, C₁-C₇alkyl, an optionally substituted C₆-C₁₀aryl, an optionally substituted C₈-C₁₀arylC₁-C₄alkyl or an optionally substituted C₁-C₁₀heteroarylC₁-C₄alkyl. In one aspect of this embodiment, the aryl or heteroaryl group of C₆-C₁₀aryl, C₆-C₁₀arylC₁-C₄alkyl, C₁-C₁₀heteroaryl or C₁-C₁₀heteroarylC₁-C₄alkyl are optionally substituted with one or more substituents independently selected from the group consisting of C₁-C₆alkyl, C₂-C₆alkenyl, C₂-C₆alkynyl, halo, C₁-C₆haloalkyl, cyano, nitro, C₁-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁵, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁵, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein W is a direct bond and R¹ is an optionally substituted C₆-C₁₀aryl or an optionally substituted C₁-C₁₀heteroaryl. In one aspect of this embodiment, the aryl or heteroaryl group of R¹ are optionally substituted with one or more substituents independently selected from the group consisting of C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₈alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁵), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁵ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₈heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein R² is hydroxy, an optionally substituted C₃-C₇cycloalkyl, haloC₁-C₄alkyl, an optionally substituted C₆-C₁₀aryl, an optionally substituted C₆-C₁₀arylC₁-C₄alkyloxy or an optionally substituted C₁-C₁₀heteroaryl. In one aspect of this embodiment, the aryl group of the arylalkyloxy, the cycloalkyl, aryl and heteroaryl are optionally substituted with one or more substituents independently selected from the group consisting of of C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein V—R² is selected from the group consisting of:

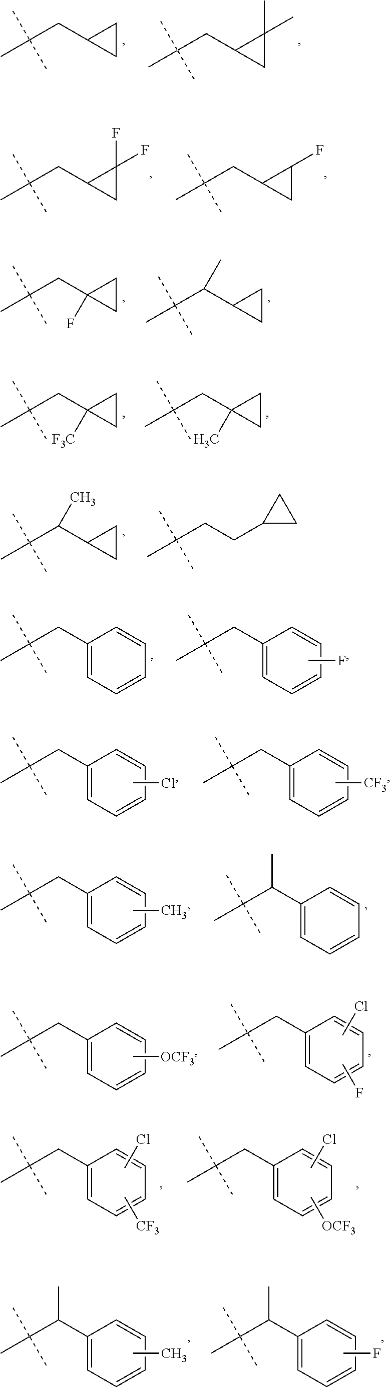

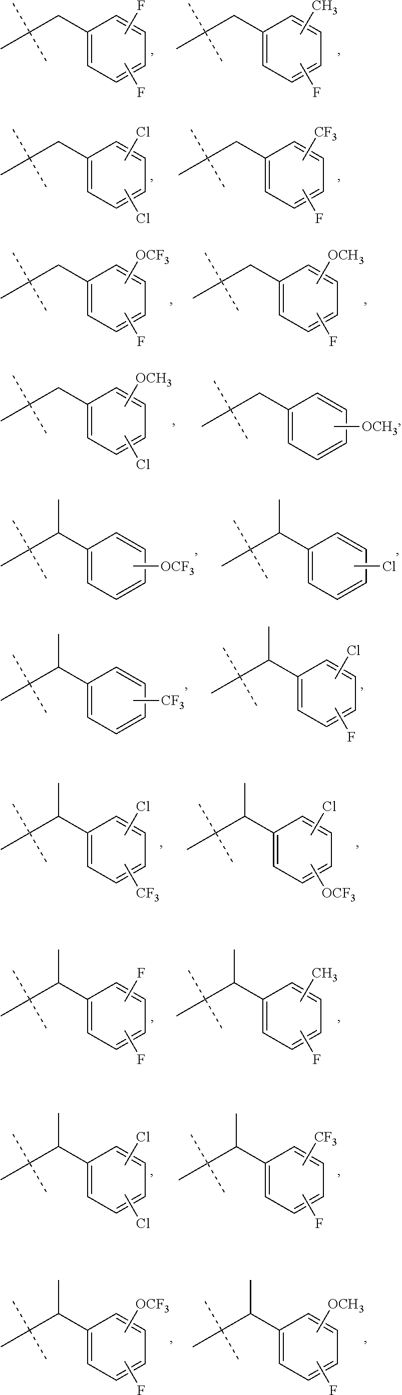

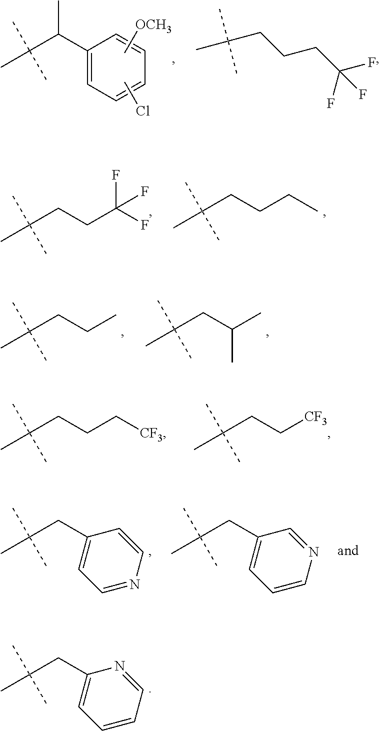

In another embodiment, the invention provides compounds of formula (I), wherein —V—R² is

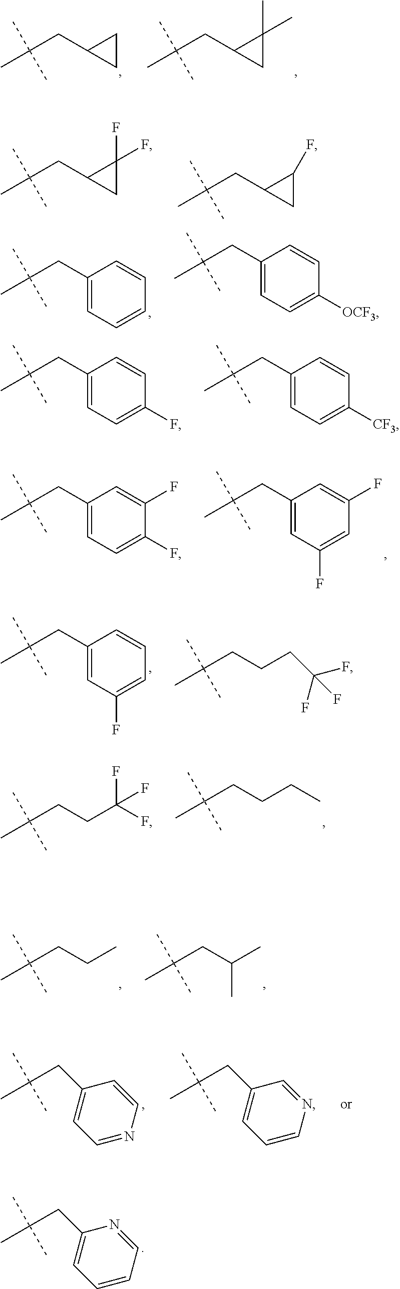

In another embodiment, the invention provides compounds of formula (I), wherein R⁵ and R^{5a} both hydrogen.

In another embodiment, the invention provides compounds of formula (I), wherein R⁵ and R^{5a} are each independently selected from hydrogen or a C₁-C₄alkyl.

In another embodiment, the invention provides compounds of formula (I), wherein R⁵ and R^{5a} are each independently selected from a C₁-C₄alkyl.

In another embodiment, the invention provides compounds of formula (I), wherein R⁷ is hydrogen.

In another embodiment, the invention provides compounds of formula (I), wherein n is 1.

In another embodiment, the invention provides compounds of formula (I), wherein n is 2.

In another embodiment, the invention provides compounds of formula (I), wherein p is 0.

In another embodiment, the invention provides compounds of formula (I), wherein p is 1 and R⁶ is selected from a C₁-C₄alkyl.

In another embodiment, the invention provides compounds of formula (I), wherein p is 2 and R⁶ is selected from a C₁-C₄alkyl.

In another embodiment, the invention provides compounds of formula (I), wherein R⁷ is hydrogen.

In another embodiment, the invention provides compounds of formula (I), wherein R⁷ is a C₁-C₄alkyl.

In another embodiment, the invention provides compounds of formula (I), wherein;
Q is

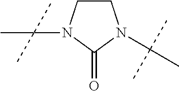

W is —N(R⁸)C(O)—;
   V is a C₁-C₆alkylene;
R¹ is hydrogen, C₁-C₇alkyl, haloC₁-C₄alkyl, an optionally substituted C₆-C₁₀aryl, an optionally substituted C₆-C₁₀arylC₁-C₄alkyl, an optionally substituted C₃-C₇cycloalkyl, an optionally substituted C₃-C₇cycloalkylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
R² is hydroxy, C₃-C₇alkyl, an optionally substituted C₃-C₇cycloalkyl, an optionally substituted C₆-C₁₀aryl, or an optionally substituted C₁-C₁₀heteroaryl;
R³ is hydrogen; and
R⁸ is hydrogen or C₁-C₄alkyl.

In one aspect of this embodiment, each aryl, cycloalkyl, heterocyclyl, or heteroaryl portion of an R¹ or R² group is independently optionally substituted with one or more substituents selected from C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein
Q is

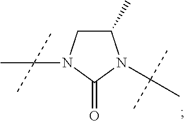

W is —N(R⁶)C(O)—;
V is a C₁-C₆alkylene;
R¹ is hydrogen, an optionally substituted aralkyl, or an optionally substituted C₁-C₁₀heteroarylC₁-C₄alkyl;
R² is C₃-C₇alkyl, haloC₁-C₄alkyl, an optionally substituted C₃-C₇cycloalkyl, an optionally substituted C₆-C₁₀aryl, or an optionally substituted C₁-C₁₀heteroaryl;
R³ is hydrogen; and
R⁸ is hydrogen.

In one aspect of this embodiment, each aryl, cycloalkyl, heterocyclyl, or heteroaryl portion of an R¹ or R² group is independently optionally substituted with one or more substituents selected from C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₁-C₆heterocyclylC₁-C₄alkyl, C₁-C₁₀heteroaryl, C₁-C₁₀heteroarylC₁-C₈alkyl, —R¹⁵—OR¹⁴, —R¹⁵—OC(O)—R¹⁴, —R¹⁵—N(R¹⁴)₂, —C(O)R¹⁴, —R¹⁵—C(O)OR¹⁴, —R¹⁵—C(O)N(R¹⁴)₂, R¹⁵—N(R¹⁴)C(O)OR¹⁶, —R¹⁵—N(R¹⁴)C(O)(R¹⁶), —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁸ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein
Q is

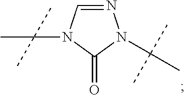

W is —N(R⁸)C(O)— or a direct bond;
V is a C₁-C₆alkylene;
R¹ is hydrogen, C₃-C₇alkyl, an optionally substituted C₆-C₁₀aryl, an optionally substituted C₆-C₁₀arylC₁-C₄alkyl, an optionally substituted C₃-C₇cycloalkyl, an optionally substituted C₃-C₇cycloalkylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;

R² is a C₁-C₄ alkyl, haloC₁-C₄alkyl, an optionally substituted C₃-C₇cycloalkyl, an optionally substituted C₆-C₁₀aryl, or an optionally substituted C₁-C₁₀heteroaryl;

R³ is hydrogen; and

R⁸ is hydrogen or C₁-C₄alkyl.

In one aspect of this embodiment, each aryl, cycloalkyl, heterocyclyl, or heteroaryl portion of an R¹ or R² group is independently optionally substituted with one or more substituents selected from C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein;
Q is

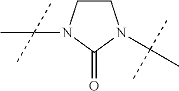

W is —N(R⁶)C(O)—;
R¹ is hydrogen, C₁-C₇alkyl, an optionally substituted C₆-C₁₀aryl, an optionally substituted C₆-C₁₀arylC₁-C₄alkyl, an optionally substituted C₃-C₇cycloalkyl, an optionally substituted C₃-C₇cycloalkylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
—V—R² is selected from the group consisting of:

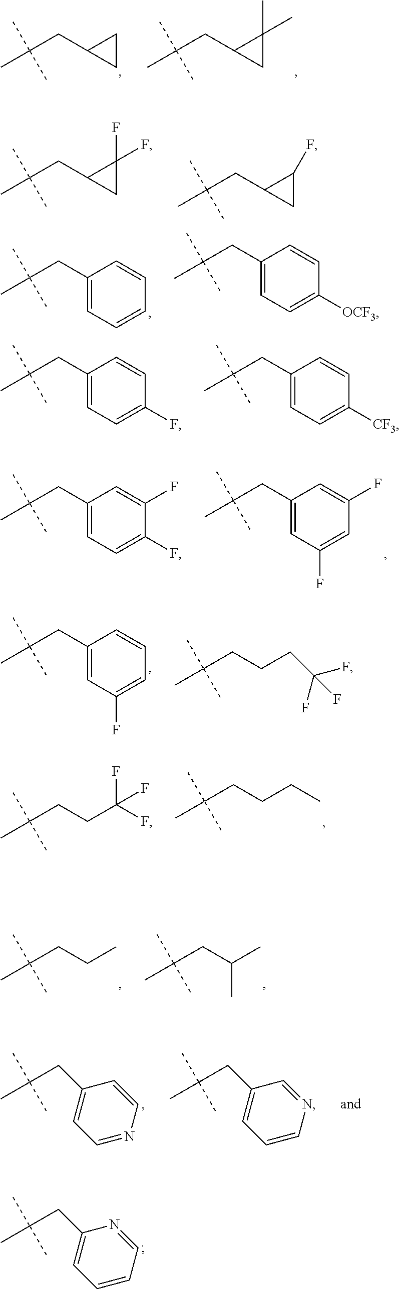

R³ is hydrogen; and
R⁸ is hydrogen or C₁-C₄alkyl.

In one aspect of this embodiment, each aryl, cycloalkyl, heterocyclyl, or heteroaryl portion of an R¹ group is optionally substituted with one or more substituents independently selected from C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₈heterocyclyl, C₂-C₈heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein
Q is

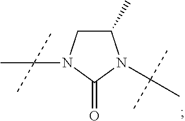

W is —N(R⁶)C(O)—;
R¹ is hydrogen, an optionally substituted aralkyl, or an optionally substituted C₁-C₁₀heteroarylC₁-C₄alkyl;
—V—R² is selected from the group consisting of:

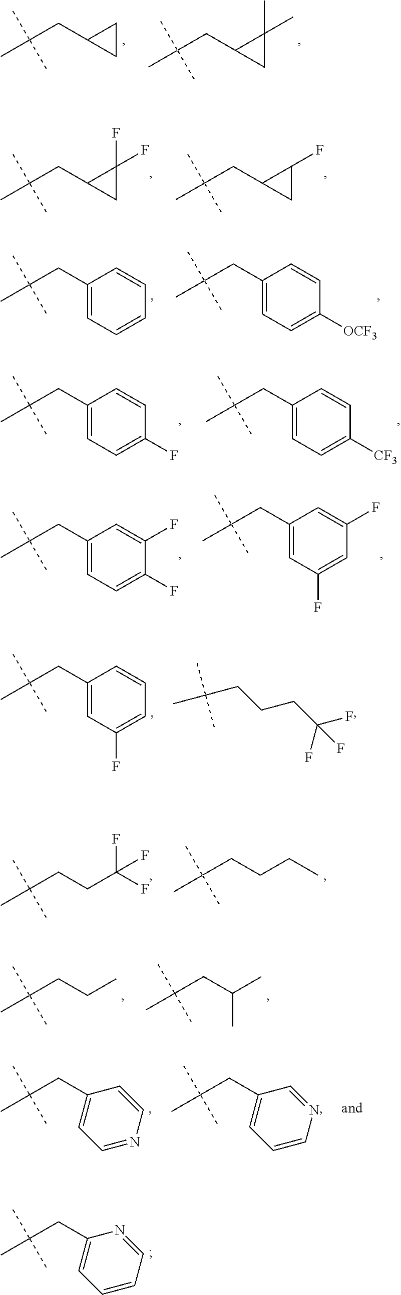

R³ is hydrogen; and
R⁸ is hydrogen.

In one aspect of this embodiment, each aryl, cycloalkyl, heterocyclyl, or heteroaryl portion of an R¹ group is optionally substituted with one or more substituents independently selected from C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein
Q is

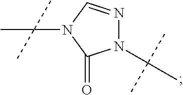

W is —N(R⁸)C(O)— or a direct bond;
R¹ is hydrogen, C₁-C₄alkyl, an optionally substituted C₆-C₁₀aryl, an optionally substituted C₆-C₁₀arylC₁-C₄alkyl, an optionally substituted C₃-C₇cycloalkyl, an optionally substituted C₃-C₇cycloalkylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
—V—R² is selected from the group consisting of:

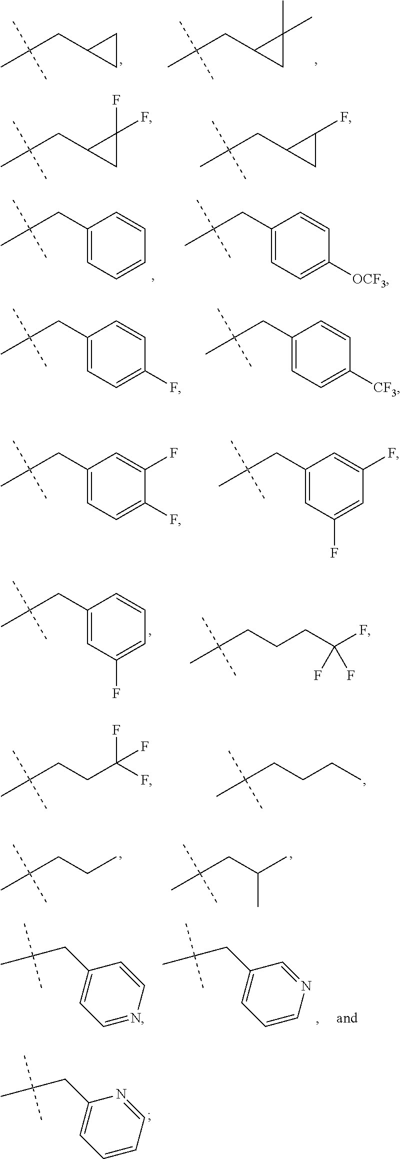

R³ is hydrogen; and
R⁸ is hydrogen or C₁-C₄alkyl.

In one aspect of this embodiment, each aryl, cycloalkyl, heterocyclyl, or heteroaryl portion of an R¹ group is optionally substituted with one or more substituents independently selected from C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR ¹⁶, —R¹⁵—S(O)ₜR¹⁶, and —R¹⁵—S(O)N(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

In another embodiment, the invention provides compounds of formula (I), wherein
W is —N(R⁸)C(O)—, and
R¹ is hydrogen, C₁-C₄alkyl,

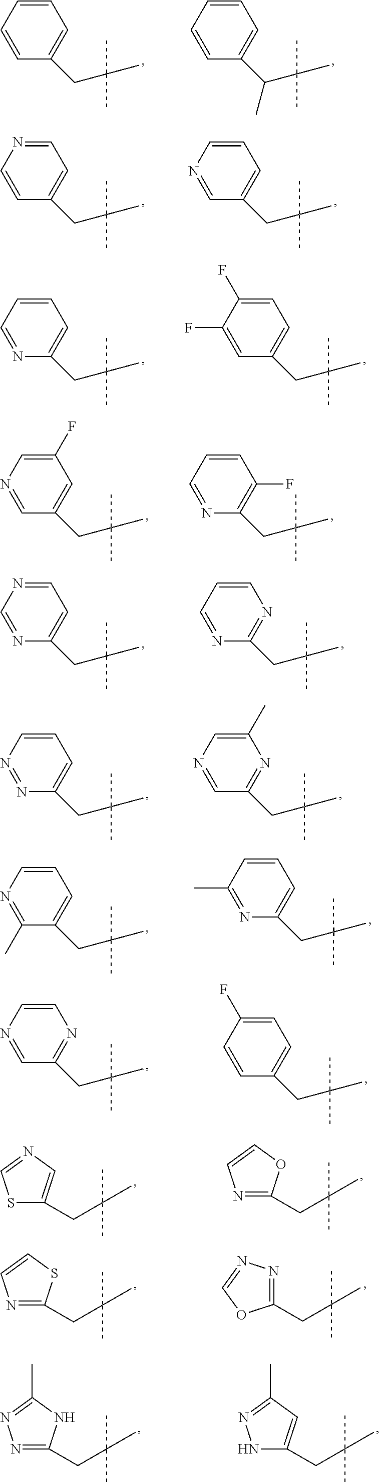

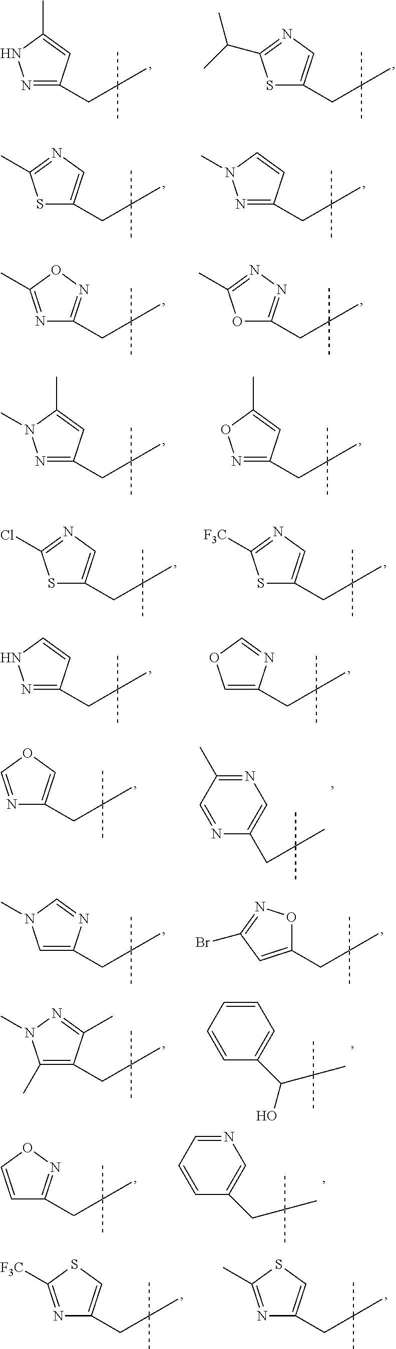

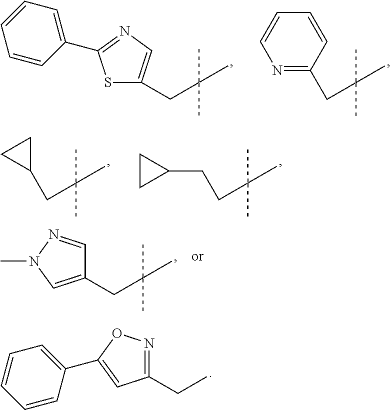

In one aspect of this embodiment, —V—R² is selected from the group consisting of:

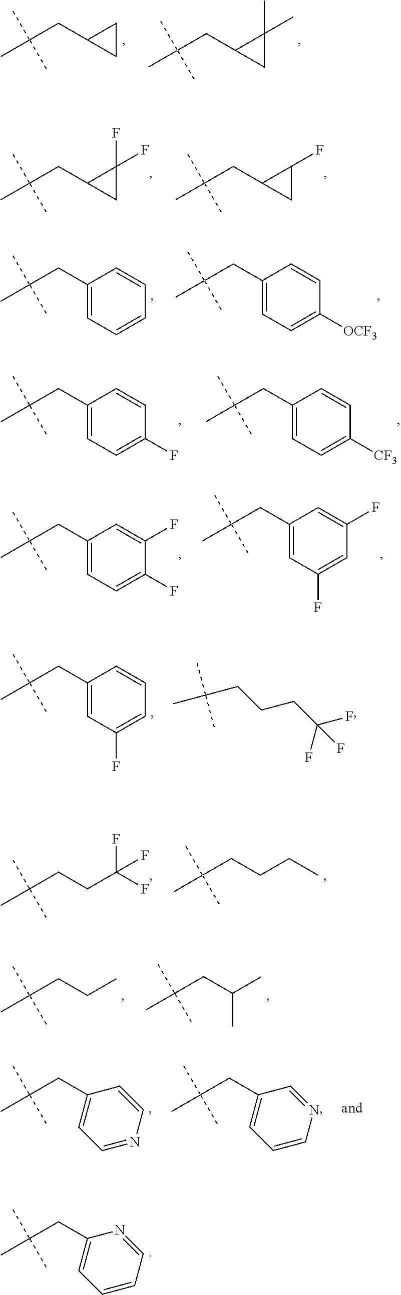

In another embodiment, the invention provides compounds of formula (I), wherein
W is a direct bond and
R₁ is

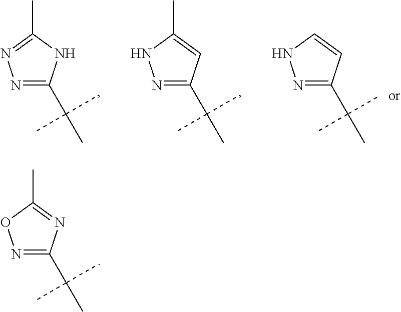

In one aspect of this embodiment, —V—R² is selected from the group consisting of:

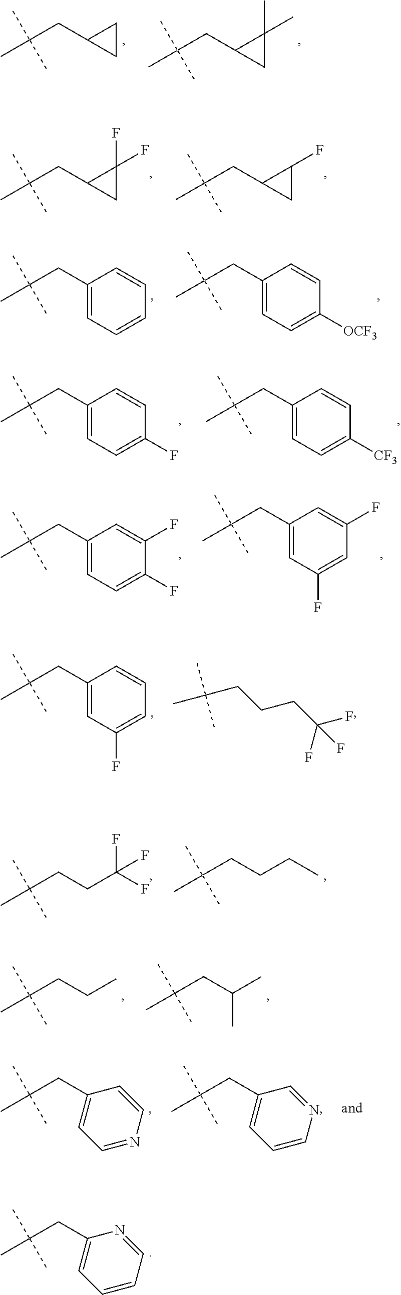

In another embodiment, the invention provides compounds of formula (I), wherein W is —N(R⁸)C(O)—, and R¹ is hydrogen, C₁-C₇alkyl,

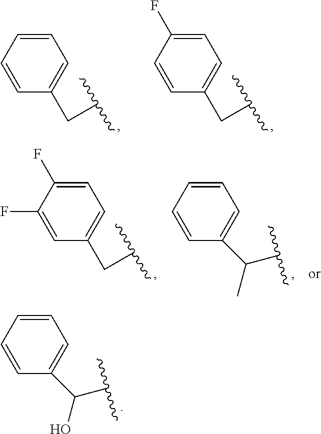

In another embodiment, the invention provides compounds of formula (I), wherein W is —N(R⁸)C(O)—, and R¹ is hydrogen, C₁-C₇alkyl,

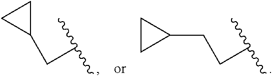 [SMILES: *CC1CC1 ; *CCC1CC1]

In another embodiment, the invention provides compounds of formula (I), wherein W is —N(R⁸)C(O)—, and R¹ is hydrogen, C₁-C₇alkyl,

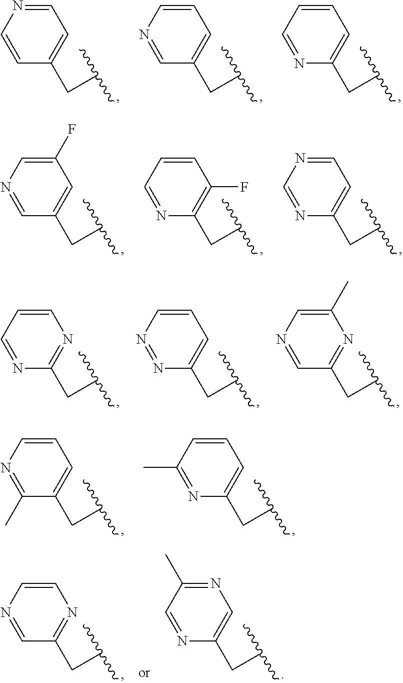

In another embodiment, the invention provides compounds of formula (I), wherein W is —N(R⁸)C(O)—, and R¹ is hydrogen, C₁-C₇alkyl,

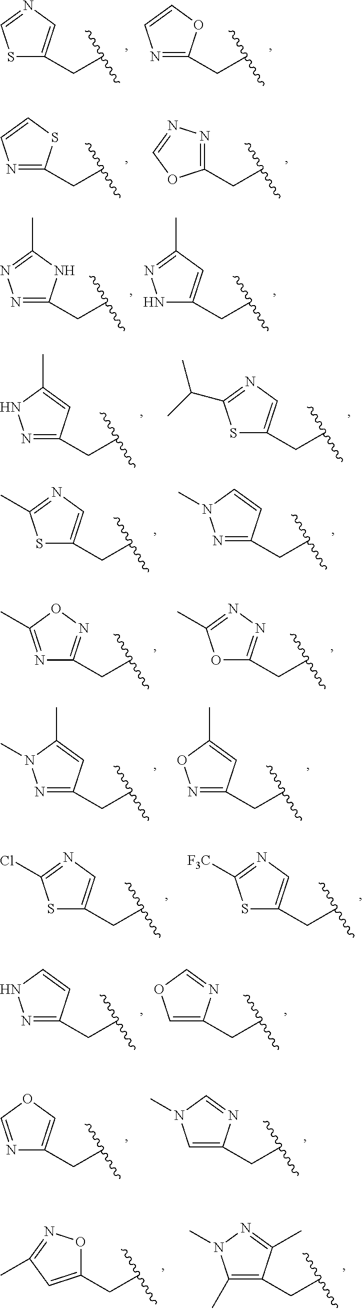

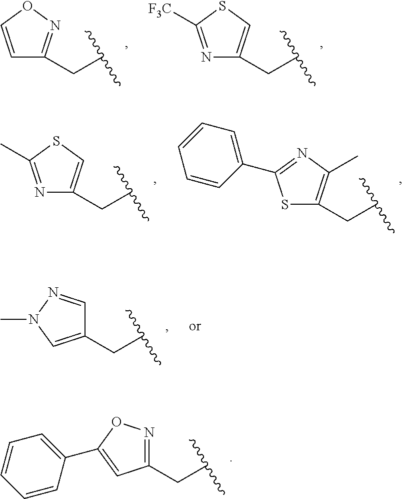

In another embodiment, the invention provides compounds of formula (I), wherein —V—R² is

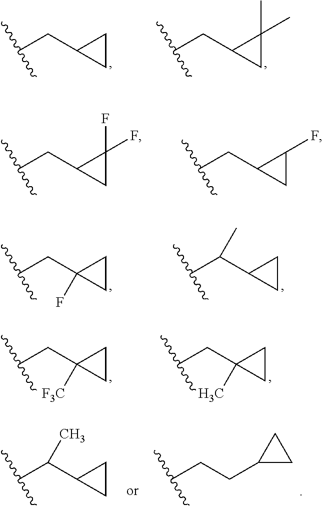

In another embodiment, the invention provides compounds of formula (I), wherein —V—R² is

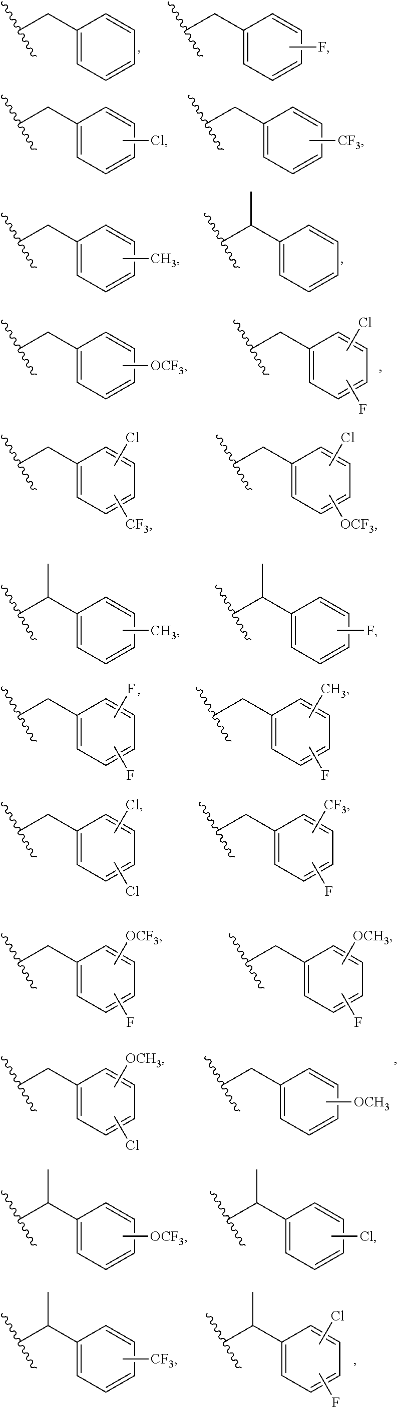

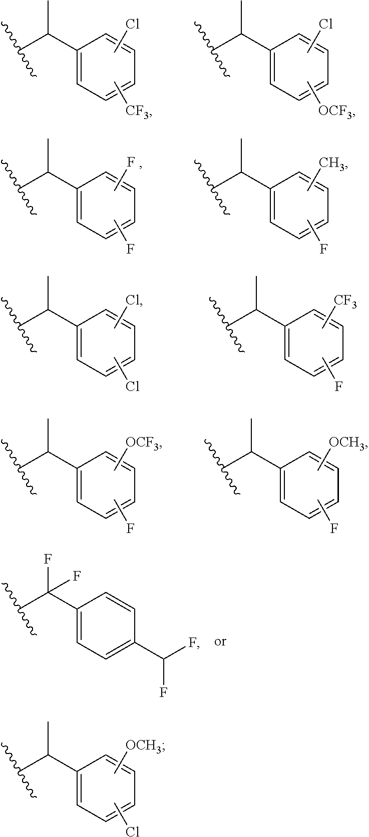

in another embodiment, the invention provides compounds of formula (I), wherein —V—R² is

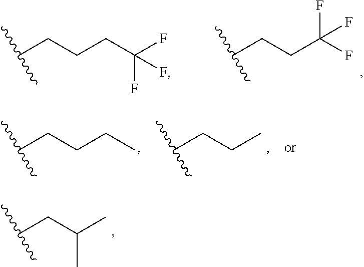

In another embodiment, the invention provides compounds of formula (I), wherein —V—R² is

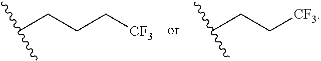 [SMILES: *CCCC(F)(F)F ; "or" ; *CCC(F)(F)F]

In another embodiment, the invention provides compounds of formula (I), wherein —V—R² is

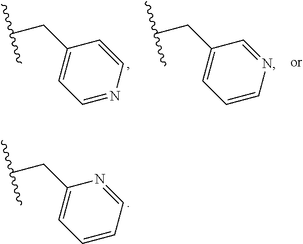

In another embodiment, the invention provides compounds of formula (I), wherein Q is

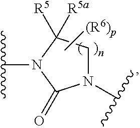

n is 1; p is 0; W is —N(H)C(O)—; R¹ is hydrogen, C₁-C₄ alkyl or an optionally substituted C₆-C₁₀arylC₁-C₄alkyl; R² is an optionally substituted C₆-C₁₀aryl; and each of R⁵ and R^{5a} is hydrogen. In one aspect of this embodiment, V is a C₁-C₄alkylene.

In another embodiment, the invention provides compounds of formula (I), wherein wherein Q is

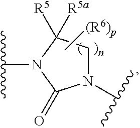

n is 1; p is 0; W is —N(R⁸)C(O)—; R¹ is an optionally substituted C₃-C₇cycloalkylC₁-C₄alkyl, an optionally substituted C₂-C₁₀heterocyclylC₁-C₄alkyl, an optionally substituted C₆-C₁₀arylC₁-C₄alkyl or an optionally substituted C₁-C₁₀heteroarylC₁-C₄alkyl; and R² is C₃-C₇alkyl, C₁-C₇alkoxy, hydroxyC₁-C₄alkyl, alkoxyC₁-C₄alkyl, an optionally substituted C₃-C₇cycloalkyl, haloC₁-C₄alkyl, an optionally substituted C₆-C₁₀aryl or an optionally substituted C₁-C₁₀heteroaryl. In one aspect of this embodiment, V is a C₁-C₄alkylene.

In another embodiment, the invention provides compounds of formula (I), wherein wherein Q is

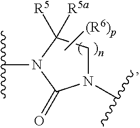

n is 1; p is 0; W is —N(H)C(O)—, R¹ is an optionally substituted C₁-C₁₀heteroarylC₁-C₄alkyl; and R² is an optionally substituted C₆-C₁₀aryl. In one aspect of this embodiment, V is a C₁-C₄alkylene.

In another embodiment, the invention provides compounds of formula (I), wherein wherein Q is

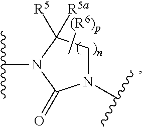

n is 1; p is 0; W is —N(H)C(O)—, R¹ is an optionally substituted C₁-C₁₀heteroarylC₁-C₄alkyl; and R² is an optionally substituted C₁-C₁₀heteroaryl. In one aspect of this embodiment, V is a C₁-C₄alkylene.

In another embodiment, the invention provides compounds of formula (I), wherein wherein Q is

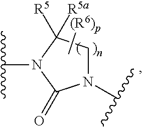

n is 1; p is 0; W is —N(H)C(O)—; R¹ is an optionally substituted C₁-C₁₀heteroarylC₁-C₄alkyl; and R² is C₃-C₇alkyl or an optionally substituted C₃-C₇cycloalkyl. In one aspect of this embodiment, V is a C₁-C₄alkylene.

In another embodiment, the invention provides compounds of formula (I), wherein wherein Q is

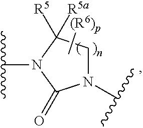

n is 1; p is 0; W is —N(H)C(O)—; R¹ is an optionally substituted C₆-C₁₀aryl C₁-C₄alkyl or an optionally substituted C₂-C₁₀heterocyclylC₁-C₄alkyl; and R² is an optionally substituted C₆-C₁₀aryl. In one aspect of this embodiment, V is a C₁-C₄alkylene.

In another embodiment, the invention provides compounds of formula (I), wherein wherein Q is

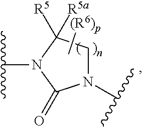

n is 1; p is 0; W is —N(H)C(O)—; R¹ is an optionally substituted C₆-C₁₀aryl C₁-C₄alkyl or an optionally substituted C₂-C₁₀heterocyclylC₁-C₄alkyl; and R² is an optionally substituted C₁-C₁₀heteroaryl. In one aspect of this embodiment, V is a C₁-C₄alkylene.

In another embodiment, the invention provides compounds of formula (I), wherein wherein Q is

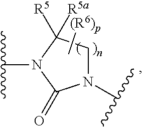

n is 1; p is 0; W is —N(H)C(O)—; R¹ is an optionally substituted C₆-C₁₀aryl C₁-C₄alkyl or an optionally substituted C₂-C₁₀heterocyclylC₁-C₄alkyl; and R² is alkyl or an optionally substituted C₃-C₇cycloalkyl. In one aspect of this embodiment, V is a C₁-C₄alkylene.

In another embodiment, the invention provides compounds of formula (I), wherein wherein Q is

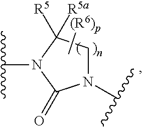

n is 1; p is 0; W is —N(H)C(O)—; R¹ is an optionally substituted C₆-C₁₀aryl or an optionally substituted C₂-C₁₀heterocyclylC₁-C₄alkyl; and R² is an optionally substituted C₁-C₁₀heteroaryl. In one aspect of this embodiment, V is a C₁-C₄alkylene.

In another embodiment, the invention provides compounds of formula (I), wherein wherein Q is

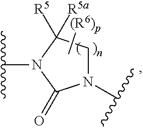

n is 1; p is 0; W is —N(H)C(O)—; R¹ is an optionally substituted C₃-C₇cycloalkylC₁-C₄alkyl; and R² is an optionally substituted C₈-C₁₀aryl. In one aspect of this embodiment, V is a C₁-C₄alkylene.

In another embodiment, the invention provides compounds of formula (I), wherein wherein Q is

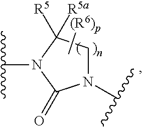

n is 1; p is 0; W is —N(H)C(O)—; R¹ is an optionally substituted C₃-C₇cycloalkylC₁-C₄alkyl; and R² is an optionally substituted C₁-C₁₀heteroaryl. In one aspect of this embodiment, V is a C₁-C₄alkylene.

In another embodiment, the invention provides compounds of formula (I), wherein wherein Q is

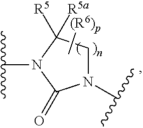

n is 1; p is 0; W is —N(H)C(O)—; R¹ is an optionally substituted C₃-C₇cycloalkylC₁-C₄alkyl; and R² is C₃-C₇alkyl or an optionally substituted C₃-C₇cycloalkyl. In one aspect of this embodiment, V is a C₁-C₄alkylene.

In another embodiment, the invention provides compounds of formula (I), wherein Wherein Q is

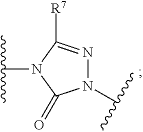

W is —N(R⁸)C(O)—; R¹ is hydrogen, C₁-C₇alkyl, C₂-C₈alkenyl, C₂-C₈alkynyl, C₁-C₇alkoxy, hydroxyC₁-C₄alkyl, alkoxyC₁-C₄alkyl, an optionally substituted C₃-C₇cycloalkyl, an optionally substituted C₃-C₇cycloalkylC₁-C₄alkyl, an optionally substituted C₆-C₁₀aryl, haloC₁-C₄alkyl, an optionally substituted C₆-C₁₀arylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl; R² is hydrogen, C₃-C₇alkyl, C₂-C₆alkenyl, C₂-C₆alkynyl, C₁-C₇alkoxy, hydroxy, hydroxyC₁-C₄alkyl, alkoxyC₁-C₄alkyl, an optionally substituted C₃-C₇cycloalkyl, an optionally substituted C₆-C₁₀aryl, haloC₁-C₄alkyl, an optionally substituted C₂-C₁₀heterocyclyl, or an optionally substituted C₁-C₁₀heteroaryl; R³ is hydrogen or C₁-C₄alkyl; and R⁷ is independently hydrogen, C₁-C₄alkyl, haloC₁-C₄alkyl or C₆-C₁₀aryl. In one aspect of this embodiment, V is a C₁-C₄alkylene.

In another embodiment, the invention provides compounds of formula (I), wherein Q is

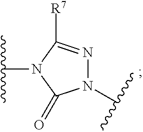

R¹ is an optionally substituted C₆-C₁₀arylC₁-C₄alkyl; and R² is an optionally substituted C₆-C₁₀aryl. In one aspect of this embodiment, V is a C₁-C₄alkylene.

In another embodiment, the invention provides compounds of formula (I), wherein Q is

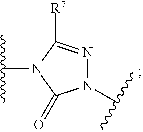

R¹ is an optionally substituted C₆-C₁₀arylC₁-C₄alkyl; and R² is an optionally substituted C₁-C₁₀heteroaryl or an optionally substituted C₃-C₇cycloalkyl. In one aspect of this embodiment, V is a C₁-C₄alkylene.

In another embodiment, the invention provides compounds of formula (I), wherein Q is

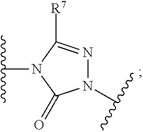

R¹ is hydrogen; and R² is an optionally substituted C₆-C₁₀aryl. In one aspect of this embodiment, V is a C₁-C₄alkylene.

In another embodiment, the invention provides compounds of formula (I), wherein Q is

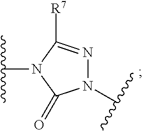

R¹ is hydrogen; and R² is a C₃-C₇alkyl or a haloC₁-C₄alkyl. In one aspect of this embodiment, V is a C₁-C₄alkylene.

In another embodiment, the invention provides compounds of formula (I), wherein Q is

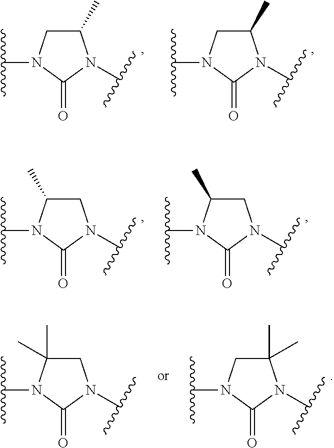

In another embodiment, the invention provides compounds of formula (I), selected from the group consisting of:
N-(3,4-Difluorobenzyl)-3-(1-(4-fluorobenzyl)-5-oxo-1H-1,2,4-triazol-4(5H)-yl)-1H-pyrazole-5-carboxamide,
3-(1-(4-Fluorobenzyl)-5-oxo-1H-1,2,4-triazol-4(5H)-yl)-N-(pyridin-3-ylmethyl)-1H-pyrazole-5-carboxamide,
N-Benzyl-3-(1-(4-fluorobenzyl)-5-oxo-1H-1,2,4-triazol-4(5H)-yl)-1H-pyrazole-5-carboxamide,
3-(1-(4-Fluorobenzyl)-5-oxo-1H-1,2,4-triazol-4 (5H)-yl)-N-methyl-1H-pyrazole-5-carboxamide,
3-(3-(4-Fluorobenzyl)-2-oxoimidazolidin-1-yl)-N-(pyridin-2-ylmethyl)-1H-pyrazole-5-carboxamide,
3-(3-(4-Fluorobenzyl)-2-oxoimidazolidin-1-yl)-N-((1-methyl-1H-pyrazol-4-yl)methyl)-1H-pyrazole-5-carboxamide,
3-(1-(4-Fluorobenzyl)-5-oxo-1H-1,2,4-triazol-4(5H)-yl)-N-(oxazol-4-ylmethyl)-1H -pyrazole-5-carboxamide,
3-(1-(4-Fluorobenzyl)-5-oxo-1H-1,2,4-triazol-4(5H)-yl)-N-((3-methyl-1H-pyrazol-5-yl)methyl)-1H-pyrazole-5-carboxamide,
3-(1-(4-Fluorobenzyl)-5-oxo-1H-1,2,4-triazol-4(5H)-yl)-N-(pyridin-4-ylmethyl)-1H-pyrazole-5-carboxamide,
3-(1-(4-Fluorobenzyl)-5-oxo-1H-1,2,4-triazol-4(5H)-yl)-N-(pyridin-2-ylmethyl)-1H-pyrazole-5-carboxamide,
3-(3-(4-Fluorobenzyl)-2-oxoimidazolidin-1-yl)-N-((5-methyl-1H-pyrazol-3-yl)methyl)-1H-pyrazole-5-carboxamide,
3-(3-(4-Fluorobenzyl)-2-oxoimidazolidin-1-yl)-N-(thiazol-2-ylmethyl)-1H-pyrazole-5-carboxamide,
N-Benzyl-3-(3-(4-fluorobenzyl)-2-oxoimidazolidin-1-yl)-1H-pyrazole-5-carboxamide,
3-(3-(4-Fluorobenzyl)-2-oxoimidazolidin-1-yl)-N-((5-methylisoxazol-3-yl)methyl)-1H-pyrazole-5-carboxamide,
3-(3-(4-Fluorobenzyl)-2-oxoimidazolidin-1-yl)-1H-pyrazole-5-carboxamide,
3-(3-(4-Fluorobenzyl)-2-oxoimidazolidin-1-yl)-N-(pyridin-3-ylmethyl)-1H-pyrazole-5-carboxamide,
3-(1-(4-Fluorobenzyl)-5-oxo-1H-1,2,4-triazol-4(5H)-yl)-1H-pyrazole-5-carboxamide, and
3-(1-(4-Fluorobenzyl)-5-oxo-1H-1,2,4-triazol-4(5H)-yl)-N-((5-methylisoxazol-3-yl)methyl)-1H-pyrazole-5-carboxamide;
or a pharmaceutically acceptable salt thereof.

In another embodiment Q, W, R¹, R², R³, R⁵, R^{5a}, R⁶, R⁷ and R⁸ groups are those defined by the Q, W, R¹, R², R³, R⁵, R^{5a}, R⁶, R⁷ and R⁸ groups, respectively, in Examples 1 to 9.8 in the Examples section below.

In another embodiment individual compounds according to the invention are those listed in Examples 1 to 9.8 in the Examples section below.

In one embodiment, the methods of the invention are directed towards the treatment and/or prevention of diseases mediated by stearoyl-CoA desaturase (SCD), especially human SCD (hSCD), preferably diseases related to dyslipidemia and disorders of lipid metabolism, and especially a disease related to elevated plasma lipid levels, cardiovascular disease, diabetes, obesity, metabolic syndrome, dermatological disorders and the like by administering an effective amount of a compound of the invention.

The present invention also relates to pharmaceutical composition containing the compounds of the invention. In one embodiment, the invention relates to a composition comprising compounds of the invention in a pharmaceutically acceptable carrier and in an amount effective to modulate triglyceride level or to treat diseases related to dyslipidemia and disorders of lipid metabolism, when administered to an animal, preferably a mammal, most preferably a human patient. In an embodiment of such composition, the patient has an elevated lipid level, such as elevated triglycerides or cholesterol, before administration of said compound of the invention and the compound of the invention is present in an amount effective to reduce said lipid level.

### Utility and Testing of the Compounds of the Invention

The present invention relates to compounds, pharmaceutical compositions and methods of using the compounds and pharmaceutical compositions for the treatment and/or prevention of diseases mediated by stearoyl-CoA desaturase (SCD), especially human SCD (hSCD), preferably diseases related to dyslipidemia and disorders of lipid metabolism, and especially a disease related to elevated plasma lipid levels, especially cardiovascular disease, diabetes, obesity, metabolic syndrome, dermatological disorders and the like, by administering to a patient in need of such treatment an effective amount of an SCD modulating, especially inhibiting, agent.

In general, the present invention provides a method for treating a patient for, or protecting a patient from developing, a disease related to dyslipidemia and/or a disorder of lipid metabolism, wherein lipid levels in an animal, especially a human being, are outside the normal range (i.e., abnormal lipid level, such as elevated plasma lipid levels), especially levels higher than normal, preferably where said lipid is a fatty acid, such as a free or complexed fatty acid, triglycerides, phospholipids, or cholesterol, such as where LDL-cholesterol levels are elevated or HDL-cholesterol levels are reduced, or any combination of these, where said lipid-related condition or disease is an SCD-mediated disease or condition, comprising administering to an animal, such as a mammal, especially a human patient, a therapeutically effective amount of a compound of the invention or a pharmaceutical composition comprising a compound of the invention wherein the compound modulates the activity of SCD, preferably human SCD1.

The compounds of the invention modulate, preferably inhibit, the activity of human SCD enzymes, especially human SCD1.

The general value of the compounds of the invention in modulating, especially inhibiting, the activity of SCD can be determined using the assay described below in Example 9.

Alternatively, the general value of the compounds in treating disorders and diseases may be established in industry standard animal models for demonstrating the efficacy of compounds in treating obesity, diabetes or elevated triglyceride or cholesterol levels or for improving glucose tolerance. Such models include Zucker obese fa/fa rats (available from Harlan Sprague Dawley, Inc. (Indianapolis, Ind.)), or the Zucker diabetic fatty rat (ZDF/GmiCrl-fa/fa) (available from Charles River Laboratories (Montreal, Quebec)), and Sprague Dawley rats (Charles Rivers), as used in models for diet-induced obesity (Ghibaudi, L. et al., (2002), Obes. Res. Vol. 10, pp. 956-963). Similar models have also been developed for mice and Lewis rat.

The compounds of the instant invention are inhibitors of delta-9 desaturases and are useful for treating diseases and disorders in humans and other organisms, including all those human diseases and disorders which are the result of aberrant delta-9 desaturase biological activity or which may be ameliorated by modulation of delta-9 desaturase biological activity.

As defined herein, an SCD-mediated disease or condition is defined as any disease or condition in which the activity of SCD is elevated and/or where inhibition of SCD activity can be demonstrated to bring about symptomatic improvements for the individual so treated. As defined herein, an SCD-mediated disease or condition includes, but is not limited to, a disease or condition which is, or is related to, cardiovascular disease, dyslipidemias (including but not limited to disorders of serum levels of triglycerides, hypertriglyceridemia, VLDL, HDL, LDL, fatty acid Desaturation Index (e.g. the ratio of 18:1/18:0 fatty acids, or other fatty acids, as defined elsewhere herein), cholesterol, and total cholesterol, hypercholesterolemia, as well as cholesterol disorders (including disorders characterized by defective reverse cholesterol transport)), familial combined hyperlipidemia, coronary artery disease, arteriosclerosis, atherosclerosis, heart disease, cerebrovascular disease (including but not limited to stroke, ischemic stroke and transient ischemic attack (TIA)), peripheral vascular disease, and ischemic retinopathy.

An SCD-mediated disease or condition also includes metabolic syndrome (including but not limited to dyslipidemia, obesity and insulin resistance, hypertension, microalbuminemia, hyperuricaemia, and hypercoagulability), Syndrome X, diabetes, insulin resistance, decreased glucose tolerance, non-insulin-dependent diabetes mellitus, Type II diabetes, Type I diabetes, diabetic complications, body weight disorders (including but not limited to obesity, overweight, cachexia, bulimia and anorexia), weight loss, wasting disorders, body mass index and leptin-related diseases. In a preferred embodiment, compounds of the invention will be used to treat diabetes mellitus and/or obesity.

An SCD-mediated disease also includes obesity related syndromes, disorders and diseases that include, but not limited to, obesity as a result of (i) genetics, (ii) diet, (iii) food intake volume, (iv) a metabolic disorder, (v) a hypothalamic disorder, (vi) age, (vii) abnormal adipose distribution, (viii) abnormal adipose compartment distribution, (ix) compulsive eating disorders, and (x) motivational disorders which include the desire to consume sugars, carbohydrates, alcohols or drugs. Symptoms associates with obesity related syndromes, disorders and diseases include, but not limited to, reduced activity. Obesity also increases the likelihood of sleep apnea, gallstones, osteoporosis and certain cancers.

As used herein, the term “metabolic syndrome” is a recognized clinical term used to describe a condition comprising combinations of Type II diabetes, impaired glucose tolerance, insulin resistance, hypertension, obesity, increased abdominal girth, hypertriglyceridemia, low HDL, hyperuricaemia, hypercoagulability and/or microalbuminemia. The American Heart Association has published guidelines for the diagnosis of metabolic syndrome, Grundy, S., et. at, (2006) Cardiol. Rev. Vol. 13, No. 6, pp. 322-327.

An SCD-mediated disease or condition also includes fatty liver, hepatic steatosis, vascular restenosis, hepatitis, non-alcoholic hepatitis, non-alcoholic steatohepatitis (NASH), alcoholic hepatitis, acute fatty liver, fatty liver of pregnancy, drug-induced hepatitis, erythrohepatic protoporphyria, iron overload disorders, hereditary hemochromatosis, hepatic fibrosis, hepatic cirrhosis, hepatoma, hepatomegaly and conditions related thereto.

An SCD-mediated disease or condition also includes biliary cholesterol crystallization and related conditions, such as but not limited to, gallstones, primary sclerosing cholangitis (PSC), progressive familial intrahepatic cholestasis (PFIC), high serum gamma-glutamyl transferase (GGT) PFIC, low-GGT PFIC (i.e. Byler disease, Byler syndrome), Caroli's disease, biliary helminthiasis, biliary strictures, choledocholithiasis, obstructive cholestasis, chronic cholestatic disease, presence of biliary sludge, and cholesterolosis of gallbladder.

An SCD-mediated disease or condition also includes but is not limited to a disease or condition which is, or is related to primary hypertriglyceridemia, or hypertriglyceridemia secondary to another disorder or disease, such as hyperlipoproteinemias, familial histiocytic reticulosis, lipoprotein lipase deficiency, apolipoprotein deficiency (such as ApoCII deficiency or ApoE deficiency), and the like, or hypertriglyceridemia of unknown or unspecified etiology.

An SCD-mediated disease or condition also includes a disorder of polyunsaturated fatty acid (PUFA) disorder, or a dermatological or skin disorder, including but not limited to eczema, acne, rosacea, skin ageing, seborrheic skin, psoriasis, keloid scar formation or prevention, diseases related to production or secretions from mucous membranes, such as monounsaturated fatty acids, wax esters, and the like. Preferably, the compounds of the invention will prevent or attenuate keloid scar formation by reduction of excessive sebum production that typically results in their formation. The investigation of the role of SCD inhibitors in the treatment of acne was advanced by the discovery that rodents lacking a functional SCD1 gene had changes to the condition of their eyes, skin, coat (Zheng Y., et al. “SCD1 is expressed in sebaceous glands and is disrupted in the asebia mouse”, Nat. Genet. (1999) 23:268-270. Miyazaki, M., “Targeted Disruption of Stearoyl-CoA Desaturase1 Gene in Mice Causes Atrophy of Sebaceous and Meibomian Glands and Depletion of Wax Esters in the Eyelid”, J. Nutr. (2001), Vol. 131, pp 2260-68., Binczek, E. et al., “Obesity resistance of the stearoyl-CoA desaturase-deficient mouse results from disruption of the epidermal lipid barrier and adaptive thermoregulation”, Biol. Chem. (2007) Vol. 388 No. 4, pp 405-18).

An SCD-mediated disease or condition also includes inflammation, sinusitis, asthma, bronchitis, pancreatitis, osteoarthritis, rheumatoid arthritis, cystic fibrosis, and premenstrual syndrome.

An SCD-mediated disease or condition also includes but is not limited to a disease or condition which is, or is related to cancer, polycystic ovary syndrome, neoplasia, malignancy, metastases, tumours (benign or malignant), carcinogenesis, hepatomas and the like.

An SCD-mediated disease or condition also includes a condition where increasing lean body mass or lean muscle mass is desired, such as is desirable in enhancing performance through muscle building. Myopathies and lipid myopathies such as carnitine palmitoyltransferase deficiency (CPT I or CPT II) are also included herein. Such treatments are useful in humans and in animal husbandry, including for administration to bovine, porcine or avian domestic animals or any other animal to reduce triglyceride production and/or provide leaner meat products and/or healthier animals.

An SCD-mediated disease or condition also includes a disease or condition that is, or is related to, neurological diseases, psychiatric disorders, multiple sclerosis, eye diseases, polycystic ovary syndrome, sleep-disordered (e.g disturbances of breathing or circadian rhythm, dysomnia, insomnia, sleep apnea, and narcolepsy), abnormal alanine transferase levels, respiratory disorders and immune disorders.

An SCD-mediated disease or condition also includes neurological diseases, including mild cognitive impairment (MCI), cerebral amyloid angipathy (CAA), down syndrome (DS), depression, schizophrenia, obsessive-compulsive disorder, and biopolar disorder.

An SCD-mediated disease or condition also includes neurodegenerative diseases, including Alzheimer's disease, Parkinson's disease, dementia with Lewy bodies, amyotrophic lateral sclerosis or Lou Gehrig's disease, Alpers' disease, Leigh's disease, Pelizaeus-Merzbacher disease, Olivopontocerebellar atrophy, Friedreich's ataxia, leukodystrophies, Rett syndrome, Ramsay Hunt syndrome type II, and Down's syndrome.

An SCD-mediated disease or condition also includes a disease or condition which is, or is related to, viral diseases or infections including but not limited to all positive strand RNA viruses, coronaviruses, SARS virus, SARS-associated coronavirus, Togaviruses, Picornaviruses, Coxsackievirus, Yellow Fever virus, Flaviviridae, ALPHAVIRUS (TOGAVIRIDAE) including Rubella virus, Eastern equine encephalitis virus, Western equine encephalitis virus, Venezuelan equine encephalitis virus, Sindbis virus, Semliki forest virus, Chikungunya virus, O'nyong'nyong virus, Ross river virus, Mayaro virus, Alphaviruses; ASTROVIRIDAE including Astrovirus, Human Astroviruses; CALICIVIRIDAE including Vesicular exanthema of swine virus, Norwalk virus, Calicivirus, Bovine calicivirus, Pig calcivirus, Hepatitis E; CORONAVIRIDAE including Coronavirus, SARS virus, Avian infectious bronchitis virus, Bovine coronavirus, Canine coronavirus, Feline infectious peritonitis virus, Human coronavirus 299E, Human coronavirus OC43, Murine hepatitis virus, Porcine epidemic diarrhea virus, Porcine hemagglutinating encephalomyelitis virus, Porcine transmissible gastroenteritis virus, Rat coronavirus, Turkey coronavirus, Rabbit coronavirus, Berne virus, Breda virus; FLAVIVIRIDAE including Hepatitis C virus, West Nile virus, Yellow Fever virus, St. Louis encephalitis virus, Dengue Group, Hepatitis G virus, Japanese B encephalitis virus, Murray Valley encephalitis virus, Central European tick-borne encephalitis virus, Far Eastern tick-borne encephalitis virus, Kyasanur forest virus, Louping ill virus, Powassan virus, Omsk hemorrhagic fever virus, Kumilinge virus, Absetarov anzalova hypr virus, ITheus virus, Rocio encephalitis virus, Langat virus, Pestivirus, Bovine viral diarrhea, Hog cholera virus, Rio Bravo Group, Tyuleniy Group, Ntaya Group, Uganda S Group, Modoc Group; PICORNAVIRIDAE including Coxsackie A virus, Rhinovirus, Hepatitis A virus, Encephalomyocarditis virus, Mengovirus, ME virus, Human poliovirus 1, Coxsackie B; POCYVIRIDAE including Potyvirus, Rymovirus, Bymovirus. Additionally it can be a disease or infection caused by or linked to Hepatitis viruses, Hepatitis B virus, Hepatitis C virus, human immunodeficiency virus (HIV) and the like. Treatable viral infections include those where the virus employs an RNA intermediate as part of the replicative cycle (hepatitis or HIV); additionally it can be a disease or infection caused by or linked to RNA negative strand viruses such as influenza and parainfluenza viruses.

The compounds identified in the instant specification inhibit the desaturation of various fatty acids (such as the C₉-C₁₀ desaturation of stearoyl-CoA), which is accomplished by delta-9 desaturases, such as stearoyl-CoA desaturase 1 (SCD1). As such, these compounds inhibit the formation of various fatty acids and downstream metabolites thereof. This may lead to an accumulation of stearoyl-CoA or palmitoyl-CoA and other upstream precursors of various fatty acids; which may possibly result in a negative feedback loop causing an overall change in fatty acid metabolism. Any of these consequences may ultimately be responsible for the overall therapeutic benefit provided by these compounds.

Typically, a successful SCD inhibitory therapeutic agent will meet some or all of the following criteria. Oral availability should be at or above 20%. Animal model efficacy is less than about 20 mg/Kg, 2 mg/Kg, 1 mg/Kg, or 0.5 mg/Kg and the target human dose is between 10 and 250 mg/70 Kg, although doses outside of this range may be acceptable. (“mg/Kg” means milligrams of compound per kilogram of body mass of the subject to whom it is being administered). The required dosage should preferably be no more than about once or twice a day or at meal times. The therapeutic index (or ratio of toxic dose to therapeutic dose) should be greater than 10. The IC₅₀ (“Inhibitory Concentration—50%”) is a measure of the amount of compound required to achieve 50% inhibition of SCD activity, over a specific time period, in an SCD biological activity assay. Any process for measuring the activity of SCD enzymes, preferably mouse or human SCD enzymes, may be utilized to assay the activity of the compounds useful in the methods of the invention in inhibiting said SCD activity. Compounds of the invention demonstrate an IC₅₀ (“Inhibitory Concentration of 50%”) in a 15 minute microsomal assay of preferably less than 10 mM, less than 5 μM, less than 2.5 μM, less than 1 μM, less than 750 nM, less than 500 nM, less than 250 nM, less than 100 nM, less than 50 nM, and most preferably less than 20 nM. Compounds of the invention may show reversible inhibition (i.e., competitive inhibition) and preferably do not inhibit other iron binding proteins.

The identification of compounds of the invention as SCD inhibitors was readily accomplished using the SCD enzyme and microsomal assay procedure described in Shanklin J. and Summerville C., Proc. Natl. Acad. Sci. USA (1991), Vol. 88, pp. 2510-2514. When tested in this assay, compounds of the invention had less than 50% remaining SCD activity at 10 μM concentration of the test compound, preferably less than 40% remaining SCD activity at 10 μM concentration of the test compound, more preferably less than 30% remaining SCD activity at 10 μM concentration of the test compound, and even more preferably less than 20% remaining SCD activity at 10 μM concentration of the test compound, thereby demonstrating that the compounds of the invention are potent inhibitors of SCD activity.

These results provide the basis for analysis of the structure-activity relationship (SAR) between test compounds and SCD. Certain-groups tend to provide more potent inhibitory compounds. SAR analysis is one of the tools those skilled in the art may employ to identify preferred embodiments of the compounds of the invention for use as therapeutic agents. Other methods of testing the compounds disclosed herein are also readily available to those skilled in the art. Thus, in addition, the determination of the ability of a compound to inhibit SCD may be accomplished in viva In one such embodiment this is accomplished by administering said chemical agent to an animal afflicted with a triglyceride (TG)- or very low density lipoprotein (VLDL)-related disorder and subsequently detecting a change in plasma triglyceride level in said animal thereby identifying a therapeutic agent useful in treating a triglyceride (-TG)- or very low density lipoprotein (VLDL)-related disorder. In such embodiment, the animal may be a human, such as a human patient afflicted with such a disorder and in need of treatment of said disorder.

In specific embodiments of such in vivo processes, said change in SCD1 activity in said animal is a decrease in activity, preferably wherein said SCD1 modulating agent does not substantially inhibit the biological activity of a delta-5 desaturase, delta-6 desaturase or fatty acid synthetase or other enzymes containing iron at the active site.

The model systems useful for compound evaluation may include, but are not limited to, the use of liver microsomes, such as from mice that have been maintained on a high carbohydrate diet, or from human donors, including persons suffering from obesity. Immortalized cell lines, such as HepG2 (from human liver), MCF-7 (from human breast cancer) and 3T3-L1 (from mouse adipocytes) may also be used. Primary cell lines, such as mouse primary hepatocytes, are also useful in testing the compounds of the invention. Where whole animals are used, mice used as a source of primary hepatocyte cells may also be used wherein the mice have been maintained on a high carbohydrate diet to increase SCD activity in mirocrosomes and/or to elevate plasma triglyceride levels (i.e., the 18:1/18:0 ratio); alternatively mice on a normal diet or mice with normal triglyceride levels may be used. Mouse models employing transgenic mice designed for hypertriglyceridemia are also available. Rabbits and hamsters are also useful as animal models, especially those expressing CETP (cholesterol ester transfer protein).

Another suitable method for determining the in vivo efficacy of the compounds of the invention is to indirectly measure their impact on inhibition of SCD enzyme by measuring a subject's Desaturation Index after administration of the compound.

“Desaturation Index” as employed in this specification means the ratio of the product over the substrate for the SCD enzyme as measured from a given tissue sample. This may be calculated using three different equations 18:1n-9/18:0 (oleic acid over stearic acid); 16:1n-7/16:0 (palmitoleic acid over palmitic acid); and/or 16:1n-7 +18:1n-7/16:0 (measuring all reaction products of 16:0 desaturation over 16:0 substrate).

Desaturation Index is primarily measured in liver or plasma triglycerides, but may also be measured in other selected lipid fractions from a variety of tissues. Desaturation Index, generally speaking, is a tool for plasma lipid profiling.

A number of human diseases and disorders are the result of aberrant SCD1 biological activity and may be ameliorated by modulation of SCD1 biological activity using the therapeutic agents of the invention.

Inhibition of SCD expression may also affect the fatty acid composition of membrane phospholipids, as well as production or levels of triglycerides and cholesterol esters. The fatty acid composition of phospholipids ultimately determines membrane fluidity, with a subsequent modulation of the activity of multiple enzymes present within the membrane, while the effects on the composition of triglycerides and cholesterol esters can affect lipoprotein metabolism and adiposity.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented.

For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

Alternatively, another format can be used to measure the effect of SCD inhibition on sebaceous gland function. In a typical study using ridnets, oral, intravenous or topical formulations of the SCD inhibitor are administered to a rodent for a period of 1 to 8 days. Skin samples are taken and prepared for histological assessment to determine sebaceous gland number, size, or lipid content. A reduction of sebaceous gland size, number or function would indicate that the SCD inhibitor would have a beneficial impact on acne vulgaris, (Clark, S. B. et al. “Pharmacological modulation of sebaceous gland activity: mechanisms and clinical applications”, Dermatol. Clin. (2007) Vol. 25, No. 2, pp 137-46. Geiger, J. M., “Retinoids and sebaceous gland activity” Dermatology (1995), Vol. 191, No. 4, pp 305-10).

### Pharmaceutical Compositions of the Invention and Administration

The present invention also relates to pharmaceutical composition containing the compounds of the invention disclosed herein. In one embodiment, the present invention relates to a composition comprising compounds of the invention in a pharmaceutically acceptable carrier and in an amount effective to modulate triglyceride level or to treat diseases related to dyslipidemia and disorders of lipid metabolism, when administered to an animal, preferably a mammal, most preferably a human patient. In an embodiment of such composition, the patient has an elevated lipid level, such as elevated triglycerides or cholesterol, before administration of said compound of the invention and the compound of the invention is present in an amount effective to reduce said lipid level.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids, such as water, saline, glycerol and ethanol, and the like. A thorough discussion of pharmaceutically acceptable carriers, diluents, and other excipients is presented in REMINGTON′S PHARMACEUTICAL SCIENCES (Mack Pub. Co., N.J. current edition).

Those skilled in the art are familiar with how to determine suitable doses of the compounds for use in treating the diseases and disorders contemplated herein.

Therapeutic doses are generally identified through a dose ranging study in humans based on preliminary evidence derived from animal studies. Doses must be sufficient to result in a desired therapeutic benefit without causing unwanted side effects for the patient. The preferred dosage range for an animal is 0.001 mg/Kg to 10,000 mg/Kg, including 0.5 mg/Kg, 1.0 mg/Kg, 2.0 mg/Kg, 5.0 mg/Kg, 10 mg/Kg and 20 mg/Kg, though doses outside this range may be acceptable. The dosing schedule may be once or twice per day, although more often or less often may be satisfactory.

Those skilled in the art are also familiar with determining administration methods (oral, intravenous, inhalation, sub-cutaneous, transdermal, topical, etc.), dosage forms, suitable pharmaceutical excipients and other matters relevant to the delivery of the compounds to a subject in need thereof.

In an alternative use of the invention, the compounds of the invention can be used in in vitro or in vivo studies as exemplary agents for comparative purposes to find other compounds also useful in treatment of, or protection from, the various diseases disclosed herein.

The pharmaceutical compositions according to the invention are those suitable for enteral, such as oral or rectal, transdermal, intravenous, intradermal, subcutanceous, intramuscular, colonical, ophthalmic, intraurethral, nasal (e.g. inhalation), intraperitoneal and parenteral administration to mammals, including man, to inhibit stearoyl-CoA desaturase, and for the treatment of conditions associated with stearoyl desaturase activity. In general, the pharmaceutical compositions comprise a therapeutically effective amount of a pharmacologically active compound of the instant invention, alone or in combination with one or more pharmaceutically acceptable carriers.

The pharmacologically active compounds of the invention are useful in the manufacture of pharmaceutical compositions comprising a therapeutically effective amount thereof in conjunction or admixture with excipients or carriers suitable for either enteral or parenteral application. For enteral or parenteral application, it is preferred to administer an effective amount of a pharmaceutical composition according to the invention as tablets or gelatin capsules. Such pharmaceutical compositions may comprise, for example, the active ingredient together with diluents (e.g., lactose, dextrose, sucrose, mannitol, sorbitol, cellulose and/or glycine), lubricants (e.g., silica, talcum, stearic acid, its magnesium or calcium salt and/or polyethyleneglycol), and for tablets also comprises binders (e.g., magnesium aluminum silicate, starch paste, gelatin, tragacanth, methylcellulose, sodium carboxymethylcellulose and/or polyvinylpyrrolidone) and disintegrants (e.g., starches, agar, alginic acid or its sodium salt) or effervescent mixtures and absorbants, colorants, flavors and sweeteners.

In another aspect of the present invention the compounds may be in the form of injectable compositions, e.g. preferably aqueous isotonic solutions or suspensions, and suppositories, which can be advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions may be prepared according to conventional mixing, granulating or coating methods, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate-controlling barrier to deliver the compound of the skin of the host at a controlled and pre-determined rate over a prolonged period of time, and means to secure the device to the skin.

The most suitable route will depend on the nature and severity of the condition being treated. Those skilled in the art are also familiar with determining administration methods, dosage forms, suitable pharmaceutical excipients and other matters relevant to the delivery of the compounds to a subject in need thereof.

The compounds of the invention may be usefully combined with one or more other therapeutic agents for the treatment of SCD-mediated diseases and conditions. Preferrably, the other therapeutic agent is selected from antidiabetics, hypolipidemic agents, anti-obesity agents, anti-hypertensive agents or inotropic agents.

Thus, an additional aspect of the present invention concerns a pharmaceutical composition comprising a therapeutically effective amount of a compound of the invention in combination with one or more other therapeutic or diagnostic agents. For example, the composition can be formulated to comprise a therapeutically effective amount of a compound of the invention as defined above, in combination with another therapeutic agent, each at an effective therapeutic dose as reported in the art. Such therapeutic agents may, for example, include insulin, insulin derivatives and mimetics; insulin secretagogues, such as the sulfonylureas, e.g., Glipizide, glyburide and Amaryl; insulinotropic sulfonylurea receptor ligands, such as meglitinides, e.g., nateglinide and repaglinide; PPARγ and/or PPARα (peroxisome proliferator-activated receptor) ligands such as MCC-555, MK767, L-165041, GW7282 or thiazolidinediones such as rosiglitazone, pioglitazone, balaglitazone, troglitazone and the like; insulin sensitizers, such as protein tyrosine phosphatase-1B (PTP-1B) inhibitors such as PTP-112; GSK3 (glycogen synthase kinase-3) inhibitors such as SB-517955, SB-4195052, SB-216763, NN-57-05441, NN-57-05445 or RXR ligands such as GW-0791, AGN-194204; sodium-dependent glucose cotransporter inhibitors, such as T-1095, glycogen phosphorylase A inhibitors, such as BAY R3401; biguanides, such as metformin; alpha-glucosidase inhibitors, such as acarbose; GLP-1 (glucagon like peptide-1), GLP-1 analogs, such as Exendin-4, and GLP-1 mimetics; DPPIV (dipeptidyl peptidase IV) inhibitors such as LAF237 (Vildagliptin) or sitagliptin; GIP and GIP mimetics such as those disclosed in WO 00/58360; PACAP and PACAP mimetics, such as those disclosed in WO 01/23420; hypolipidemic agents, such as 3-hydroxy-3-methyl-glutaryl coenzyme A (HMG-CoA) reductase inhibitors, e.g., lovastatin, pitavastatin, simvastatin, pravastatin, cerivastatin, mevastatin, velostatin, fluvastatin, dalvastatin, atorvastatin, rosuvastatin, fluindostatin and rivastatin, squalene synthase inhibitors or FXR (farnesoid X receptor) and LXR (liver X receptor) ligands, cholestyramine, fibrates, nicotinic acid and aspirin; anti-obesity agents, such as orlistat, anti-hypertensive agents, inotropic agents and hypolipidemic agents, e.g., loop diuretics, such as ethacrynic acid, furosemide and torsemide; angiotensin converting enzyme (ACE) inhibitors, such as benazepril, captopril, enalapril, fosinopril, lisinopril, moexipril, perinodopril, quinapril, ramipril and trandolapril; inhibitors of the Na-K-ATPase membrane pump, such as digoxin; neutralendopeptidase (NEP) inhibitors; ACE/NEP inhibitors, such as omapatrilat, sampatrilat and fasidotril; angiotensin II antagonists, such as candesartan, eprosartan, irbesartan, losartan, telmisartan and valsartan, in particular valsartan; β-adrenergic receptor blockers, such as acebutolol, atenolol, betaxolol, bisoprolol, metoprolol, nadolol, propranolol, sotalol and timolol; inotropic agents, such as digoxin, dobutamine and milrinone; calcium channel blockers, such as amlodipine, bepridil, diltiazem, felodipine, nicardipine, nimodipine, nifedipine, nisoldipine and verapamil. Other specific antidiabetic compounds are described by Patel Mona (Expert Opin Investig Drugs. (2003) April; 12 (4):623-33) in the FIGS. 1 to 7. A compound of the present invention may be administered either simultaneously, before or after the other active ingredient, either separately by the same or different route of administration or together in the same pharmaceutical formulation.

The structure of the active agents identified by code numbers (nos.), generic or trade names may be taken from the actual edition of the standard compendium “The Merck Index” or from databases, e.g. Patents International (e.g. IMS World Publications).

In another aspect is the use of the pharmaceutical composition as described above for production of a medicament for the treatment of SCD-mediated disease or condition.

in another aspect is the use of a pharmaceutical composition or combination as described above for the preparation of a medicament for the treatment of conditions associated with stearoyl-CoA desatruase activity.

In another aspect is a pharmaceutical composition as described above for the treatment of conditions associated with the inhibition of stearoyl-CoA desaturase.

### Preparations of Compounds

It is understood that in the following description, combinations of substituents and/or variables of the depicted formulae are permissible only if such contributions result in stable compounds.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters.

Protecting groups may be added or removed in accordance with standard techniques, which are well-known to those skilled in the art and as described herein. The use of protecting groups is described in detail in Green, T. W. and P. G. M. Wuts, Protective Groups in Organic Synthesis (2006), 4^{th} Ed., Wiley. The protecting group may also be a polymer resin such as a Wang resin or a 2-chlorotrityl-chloride resin.

It will also be appreciated by those skilled in the art, although such protected derivatives of compounds of this invention may not possess pharmacological activity as such, they may be administered to a mammal and thereafter metabolized in the body to form compounds of the invention which are pharmacologically active. Such derivatives may therefore be described as “prodrugs”. All prodrugs of compounds of this invention are included within the scope of the invention.

The following reaction schemes illustrate methods to make compounds of this invention. It is understood that one skilled in the art would be able to make these compounds by similar methods or by methods known to one skilled in the art. In general, starting components may be obtained from sources such as Sigma Aldrich, Lancaster Synthesis, Inc., Maybridge, Matrix Scientific, TCI, and Fluorochem USA, etc. or synthesized according to sources known to those skilled in the art (see, e.g., Advanced Organic Chemistry: Reactions, Mechanisms, and Structure, 5th edition (Wiley, December 2000)) or prepared as described in this invention. R¹, R², R³, R⁵, R^{5a}, R⁶, R⁷, R⁸, W and V are defined as in the Specification unless specifically defined. R′ is a protecting group.

In general, the cyclized urea compounds of Formula (I) of this invention can be synthesized following the general procedure as described in Scheme where Q is

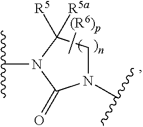

R⁵, R^{5a} and R⁶ are hydrogen, W is —N(R⁶)C(O)— and V is an alkylene.

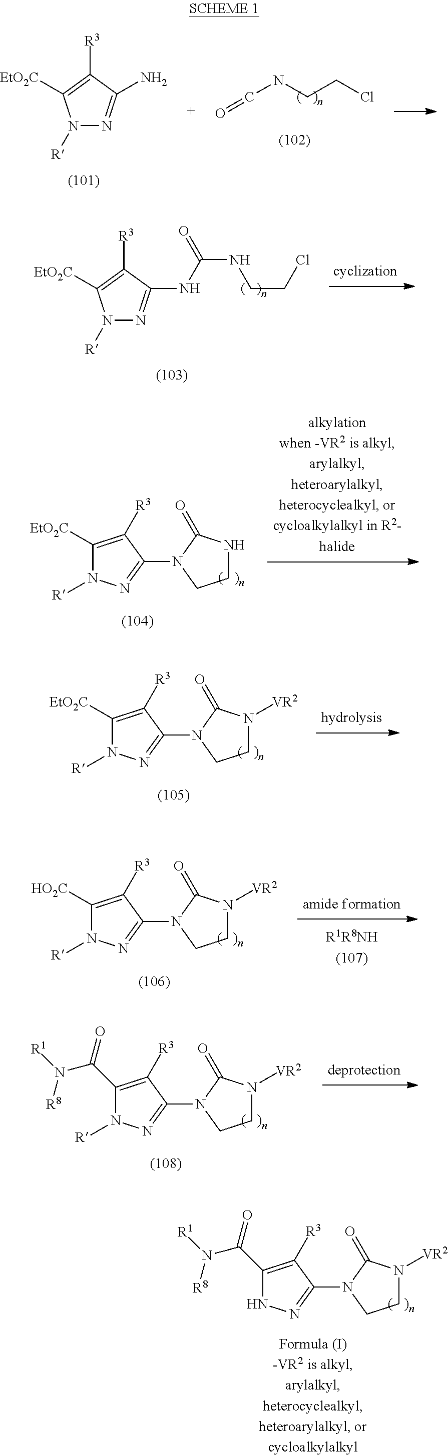

The starting materials for the above reaction scheme are commercially available or can be prepared according to methods known to one skilled in the art or by methods disclosed herein. In general, the compounds of the invention are prepared in the above reaction scheme as follows:

The aminopyrazole compound (101) reacts with isocyanate (102) to generate compound (103) which undergoes intramolecular cyclization in the presence of a base, such as, but not limited to, potassium carbonate, to afford the cyclized compound (104). Compound (104) reacts with an alkyl halide, an arylalkyl halide, a heterocyclylalkyl halide, a cycloalkyl halide, or a heteroarylalkyl halide under alkylation conditions to generate compound (105) where —VR² is an alkyl, an arylalkyl, a heterocyclylalkyl, a cycloalkyl, or a heteroarylalkyl. Compound (105) undergoes standard hydrolysis known to one skilled in the art to generate compound (106). Compound (106) then undergoes a standard amide formation reaction with an amine compound (107) to afford the compound (108). Removal of the protecting group R¹ generates the compound of Formula (I) of the invention where R² is alkyl, arylalkyl, heterocyclylalkyl, cycloalkylalkyl or heteroarylalkyl, Q is

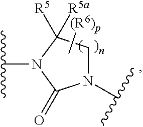

R⁵, R^{5a} and R⁸ are hydrogen, W is —N(R⁸)C(O)— and V is an alkylene.

Alternatively, the triazolone compounds of Formula (1) of this invention can be synthesized following the general procedure as described in Scheme 2 where Q is

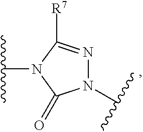

R⁷ is hydrogen, W is —N(R⁸)C(O)— and V is an alkylene.

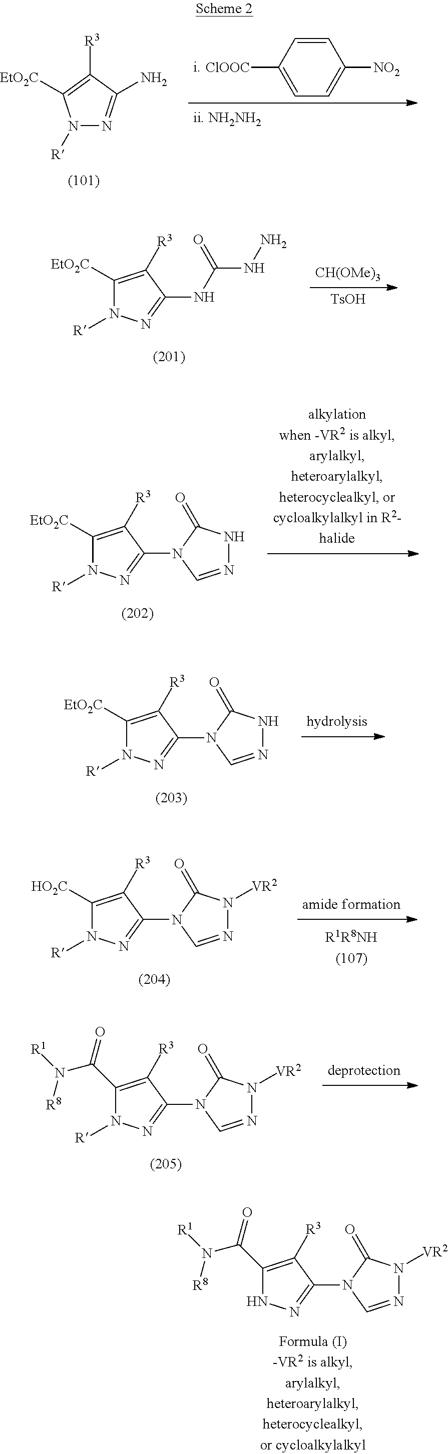

The starting materials for the above reaction scheme are commercially available or can be prepared according to methods known to one skilled in the art or by methods disclosed herein. In general, the compounds of the invention are prepared in the above reaction scheme as follows:

The aminopyrazole compound (101) reacts with chloroformate and then hydrazine to generate compound (201) which is cyclized using trimethyl orthoformate in the presence of p-toluenesulfonic acid to afford the cyclized triazolone compound (202). Compound (202) reacts with an alkyl halide, an arylalkyl halide, a heterocyclylalkyl halide, a cycloalkyl halide, or a heteroarylalkyl halide under alkylation conditions to generate compound (203) where —VR² is an alkyl, an arylalkyl, a heterocyclylalkyl, a cycloalkyl, or a heteroarylalkyl. Compound (203) undergoes standard hydrolysis known to one skilled in the art to generate compound (204). Compound (204) then undergoes a standard amide formation reaction with an amine compound to afford the compound (205). Removal of the protecting group R¹ affords the compound of Formula (I) of the invention where R² is an alkyl, an arylalkyl, a heterocyclylalkyl, a cycloalkyl, or a heteroarylalkyl, where Q is

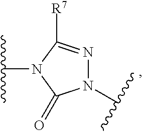

R⁷ is hydrogen, W is —N(R⁶)C(O)— and V is a direct bond.

### PREPARATION 1

### Preparation of methyl 1-(4-methoxybenzyl)-3-(5-oxo-1H-1,2,4-triazol-4(5H)-yl)-1H-pyrazole-5-carboxylate

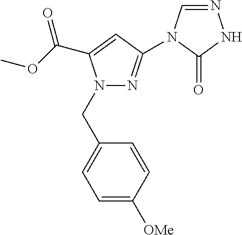 [SMILES: COC(=O)c1cc(-n2cn[nH]c2=O)nn1Cc1ccc(OC)cc1]

1. To a solution of 3-nitro-1H-pyrazole-5-carboxylic acid (2.00 g, 12.73 mmol) in methanol (80 mL) was added thionyl chloride (1.00 mL, 13.70 mmol). The reaction mixture was heated to reflux for 18 hours. The solvent was removed in vacuo, and the residue was dissolved in ethyl acetate and washed with saturated sodium bicarbonate, water and brine. The organic phase was dried over anhydrous sodium sulfate and filtered. The filtrate was concentrated in vacuo to afford methyl 3-nitro-1H-pyrazole-5-carboxylate in 81% yield (1.77 g): ¹H NMR (300 MHz, CDCl₃) δ 9.97 (br s, 1H), 7.39 (s, 1H), 3.99 (s, 3H); MS (ES+) m/z 171.0 (M).

2. To a suspension of methyl 3-nitro-1H-pyrazole-5-carboxylate (1.77 g, 10.35 mmol) and potassium carbonate (2.06 g, 14.91 mmol) in tetrahydrofuran (100 mL) was added 4-methoxybenzyl bromide (1.58 mL, 10.93 mmol). The reaction mixture was stirred for 2 hours at ambient temperature and filtered. The filtrate was concentrated in vacuo to afford methyl 1-(4-methoxybenzyl)-3-nitro-1H-pyrazole-5-carboxylate in 99% yield (3.00 g): ¹H NMR (300 MHz, CDCl₃) δ 7.37-7.32 (m, 3H), 6.86-6.81 (m, 2H), 5.75 (s, 2H), 3.90 (s, 3H), 3.76 (s, 3H); MS (ES+) m/z 292.2 (M+1).

3. A suspension of methyl 1-(4-methoxybenzyl)-3-nitro-1H-pyrazole-5-carboxylate (3.00 g, 10.27 mmol) and 10% Pd/C (0.5 g) in methanol (50 mL) was hydrogenated for 17 hours at ambient temperature and filtered through a pad of celite. The filtrate was concentrated in vacuo to afford methyl 3-amino-1-(4-methoxybenzyl)-1H-pyrazole-5-carboxylate in 99% yield (2.81 g): ¹H NMR (300 MHz, CDCl₃) δ 7.21-7.18 (m, 2H), 6.86-6.79 (m, 2H), 6.14 (s, 1H), 5.48 (s, 2H), 5.28 (s, 2H), 3.81 (s, 3H), 3.75 (s, 3H); MS (ES+) m/z 262.2 (M+1).

4. To a solution of methyl 3-amino-1-(4-methoxybenzyl)-1H-pyrazole-5-carboxylate (2.67 g, 10.21 mmol) and pyridine (1.4 mL, 17.30 mmol) in dichloromethane (100 mL) was added 4-nitrophenyl chloroformate (2.70 g, 13.33 mmol) at 0° C. The reaction mixture was stirred at ambient temperature for 3 hours, and then hydrazine monohydrate (3.0 mL, 59.90 mmol) was added. The reaction mixture was stirred at ambient temperature for 1 hour and washed with water and brine. The organic phase was dried over anhydrous sodium sulfate and filtered. The filtrate was concentrated in vacuo to afford methyl 3-(hydrazinecarboxamido)-1-(4-methoxybenzyl)-1H-pyrazole-5-carboxylate in 87% yield (2.85 g): MS (ES+) m/z 320.2 (M+1).

5. A solution of methyl 3-(hydrazinecarboxamido)-1-(4-methoxybenzyl)-1H-pyrazole-5-carboxylate (2.00 g, 6.26 mmol), trimethyl orthoformate (0.9 mL, 8.20 mmol) and p-toluenesulfonic acid monohydrate (20 mg) in methanol (50 mL) was subjected to microwave irradiation for 10 minutes at 90° C. The solvent was removed in vacuo and the residue was washed with saturated sodium bicarbonate and water and dried in vacuo to afford methyl 1-(4-methoxybenzyl)-3-(5-oxo-1H-1,2,4-triazol-4(5H)-yl)-1H-pyrazole-5-carboxylate in 96% yield (1.98 g): ¹H NMR (300 MHz, CDCl₃) δ 9.55 (br s, 1H), 8.05 (s, 1H), 7.30 (s, 1H), 7.28-7.21 (m, 2H), 6.86-6.78 (m, 2H), 5.65 (s, 2H), 3.87 (s, 3H), 3.76 (s, 3H); MS (ES+) m/z 352.1 (M+23).

### PREPARATION 2

### Preparation of methyl 1-(4-methoxybenzyl)-3-(2-oxoimidazolidin-1-yl)-1H-pyrazole-5-carboxylate

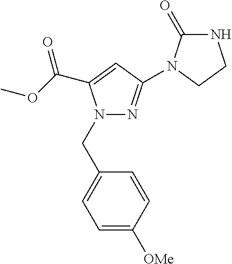 [SMILES: COC(=O)c1cc(N2CCNC2=O)nn1Cc1ccc(OC)cc1]

1. To a solution of methyl 3-amino-1-(4-methoxybenzyl)-1H-pyrazole-5-carboxylate (0.26 g, 1.00 mmol) in dichloromethane (4 mL) was added 2-chloroethylisocyanate (0.13 g, 1.20 mmol) and N,N-diisopropylethylamine (0.16 g, 1.20 mmol) at ambient temperature. The reaction mixture was stirred at ambient temperature for 3 hours. The white precipitate was filtered and washed with dichloromethane (10 mL) to afford methyl 3-(3-(2-chloroethyl)ureido)-1-(4-methoxybenzyl)-1H-pyrazole-5-carboxylate as a white solid in 73% yield (0.27 g): ¹H NMR (300 MHz, DMSO-d₆) δ 9.20 (s, 1H), 7.13 (d, J=8.7 Hz, 2H), 6.87 (d, J=8.7 Hz, 2H), 6.81 (s, 1H), 6.63 (t, J=5.2 Hz, 1H), 5.49 (s, 2H), 3.81 (s, 3H), 3.71 (s, 3H), 3.64 (t, J=6.1 Hz, 2H), 3.45-3.39 (m, 2H); MS (ES+) m/z 388.7 (M+23), 390.6 (M+23).

2. To a suspended mixture of 3-(3-(2-chloroethypureido)-1-(4-methoxybenzyl)-1H-pyrazole-5-carboxylate (0.10 g, 0.27 mmol) in N,N-dimethylformamide (9 mL) was added sodium hydride (60% in mineral oil, 0.013 g, 0.327 mmol) at 0° C. The mixture was stirred at ambient temperature for 16 hours, quenched with 25% ammonium chloride aqueous solution and extracted with dichloromethane. The organic layer was dried over anhydrous sodium sulfate and filtered. The filtrate was concentrated in vacuo and the residue was purified by column chromatography using 1-8% methanol in dichloromethane as an eluent to afford methyl 1-(4-methoxybenzyl)-3-(2-oxoimidazolidin-1-yl)-1H-pyrazole-5-carboxylate as a white solid in 57% yield (0.05 g): ¹H NMR (300 MHz, CDCl₃) δ 7.22-7.19 (m, 3H), 6.81 (d, J=8.5 Hz, 2H), 5.59 (s, 2H), 4.80 (br s, 1H), 4.05-3.99 (m, 2H), 3.83 (s, 3H), 3.76 (s, 3H), 3.61-3.55 (m, 2H); MS (ES+) m/z 352.8 (M+23).

### PREPARATION 3

### Preparation of methyl 3-(1-(4-fluorobenzyl)-5-oxo-1H-1,2,4-triazol-4(5H)-yl)-1-(4-methoxybenzyl)-1H-pyrazole-5-carboxylate

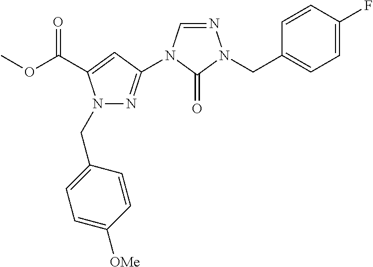 [SMILES: COC(=O)c1cc(-n2cnn(Cc3ccc(F)cc3)c2=O)nn1Cc1ccc(OC)cc1]

A mixture of methyl 1-(4-methoxybenzyl)-3-(5-oxo-1H-1,2,4-triazol-4(5H)-yl)-1H-pyrazole-5-carboxylate (4.48 g, 13.6 mmol), p-fluorobenzylbromide (3.21 g, 17.0 mmol) and potassium carbonate (2.82 g, 20.4 mmol) in acetone (450 mL) was refluxed for 3.5 hours. The hot mixture was filtered and washed with acetone. The filtrate was concentrated under reduced pressure to give a light yellow solid, which was crystallized from acetone and diethyl ether to afford methyl 3-(1-(4-fluorobenzyl)-5-oxo-1H-1,2,4-triazol-4(5H)-yl)-1-(4-methoxybenzyl)-1H-pyrazole-5-carboxylate as a white solid (5.90 g, 98%): mp 139-140° C.; MS (ES+) m/z 459.9 (M+23).

### PREPARATION 4

### Preparation of 3-(1-(4-fluorobenzyl)-5-oxo-1H-1,2,4-triazol-4(5H)-yl)-1-(4-methoxybenzyl)-1H-pyrazole-5-carboxylic acid

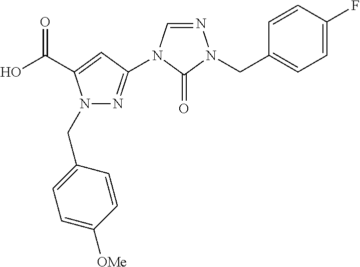 [SMILES: COc1ccc(Cn2nc(-n3cnn(Cc4ccc(F)cc4)c3=O)cc2C(=O)O)cc1]

A mixture of methyl 3-(1-(4-fluorobenzyl)-5-oxo-1H-1,2,4-triazol-4(5H)-yl)-1-(4-methoxybenzyl)-1H-pyrazole-5-carboxylate (3.80 g, 8.69 mmol) and sodium hydroxide (1 N solution, 18.2 mL, 18.2 mmol) in ethanol (100 mL) was refluxed for 2 hours, concentrated to half the original volume. The pH of the resulting solution was adjusted to 2-3 with 1 N hydrochloric acid. The solid was collected and washed with water and diethyl ether to afford 3-(1-(4-fluorobenzyl)-5-oxo-1H-1,2,4-triazol-4(5H)-yl)-1-(4-methoxybenzyl)-1H-pyrazole-5-carboxylic acid as a white solid (3.5 g, 95%): mp 205-207° C.; ¹H NMR (300 MHz, DMSO-d₆) δ 13.83 (s, 1H), 8.44 (s, 1H), 7.38-7.23 (m, 2H), 7.21-7.16 (m, 4H), 7.11 (s, 1H), 6.89-6.86 (m, 2H), 5.64 (s, 2H), 4.94 (s, 2H), 3.71 (s, 3H); MS (ES+) m/z 445.9 (M+23).

### PREPARATION 5

### Preparation of methyl 3-(3-(4-fluorobenzyl)-2-oxoimidazolidin-1-yl)-1-(4-methoxybenzyl)-1H-pyrazole-5-carboxylate

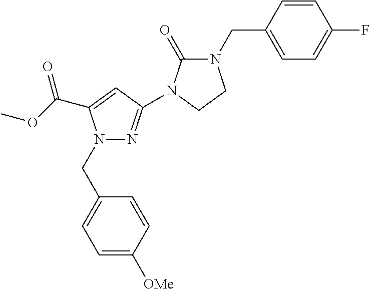 [SMILES: COC(=O)c1cc(N2CCN(Cc3ccc(F)cc3)C2=O)nn1Cc1ccc(OC)cc1]

A suspended mixture of 1-(4-methoxybenzyl)-3-(2-oxoimidazolidin-1-yl)-1H-pyrazole-5-carboxylate (0.95 g, 2.88 mmol), p-fluorobenzyl bromide (0.82 g, 4.31 mmol) and cesium carbonate (1.87 g, 5.75 mmol) in acetone (200 mL) was refluxed for 16 hours. Additional p-fluorobenzyl bromide (0.82 g, 4.31 mmol) and cesium carbonate (1.87 g, 5.75 mmol) were added and the reaction mixture was refluxed for an additional 16 hours. The hot mixture was filtered and washed with acetone. The filtrate was concentrated in vacuo to afford a light yellow solid which was purified by column chromatography using 30-50% ethyl acetate in hexane as an eluent to afford methyl 3-(3-(4-fluorobenzyl)-2-oxoimidazolidin-1-yl)-1-(4-methoxybenzyl)-1H-pyrazole-5-carboxylate as a clear oil in 48% yield (0.60 g): ¹H NMR (300 MHz, CDCl₃) δ 7.27-7.14 (m, 5H), 7.04-6.97 (m, 2H), 6.80-6.78 (m, 2H), 5.56 (s, 2H), 4.40 (s, 2H), 3.90-3.85 (m, 2H), 3.82 (s, 3H), 3.75 (s, 3H), 3.37-3.32 (m, 2H); MS (ES+) in& 438.7 (M+1).

### PREPARATION 6

### Preparation of 3-(3-(4-fluorobenzyl)-2-oxoimidazolidin-1-yl)-1-(4-methoxybenzyl)-1H-pyrazole-5-carboxylic acid

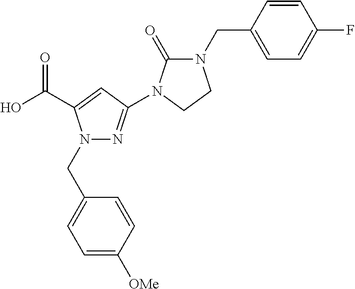 [SMILES: COc1ccc(Cn2nc(N3CCN(Cc4ccc(F)cc4)C3=O)cc2C(=O)O)cc1]

A mixture of 3-(3-(4-fluorobenzyl)-2-oxoimidazolidin-1-yl)-1-(4-methoxybenzyl)-1H-pyrazole-5-carboxylate (0.61 g, 1.39 mmol) and sodium hydroxide (1 N solution, 2.9 mL, 2.92 mmol) in ethanol (15 mL) was refluxed for 1 hour. The reaction mixture was concentrated to half of its original volume and the pH was adjusted to 1 to 2 with 3 N hydrochloric acid solution. The solid was collected by filtration and washed with water and diethyl ether to afford 3-(3-(4-fluorobenzyl)-2-oxoimidazolidin-1-yl)-1-(4-methoxybenzyl)-1H-pyrazole-5-carboxylic acid as a white solid in 83% yield (0.49 g): ¹H NMR (300 MHz, DMSO-d₆) δ 13.47 (s, 1H), 7.35-7.31 (m, 2H), 7.21-7.15 (m, 2H), 7.10 (d, J=8.7 Hz, 2H), 7.03 (s, 1H), 6.86 (d, J=8.7 Hz, 2H), 5.54 (s, 2H), 4.36 (s, 2H), 3.80-3.74 (m, 2H), 3.71 (s, 3H), 3.87-3.32 (m, 2H); MS (ES+) m/z 446.8 (M+23).

### PREPARATION 7

### Preparation of N-(3,4-difluorobenzyl)-3-(1-(4-fluorobenzyl)-5-oxo-1H-1,2,4-triazol-4(5H)-yl)-1-(4-methoxybenzyl)-1H-pyrazole-5-carboxamide

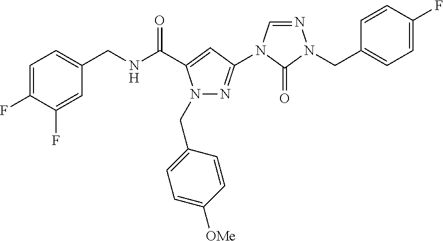 [SMILES: COc1ccc(Cn2nc(-n3cnn(Cc4ccc(F)cc4)c3=O)cc2C(=O)NCc2ccc(F)c(F)c2)cc1]

To a solution of 3-(1-(4-fluorobenzyl)-5-oxo-1H-1,2,4-triazol-4(5H)-yl)-1-(4-methoxybenzyl)-1H-pyrazole-5-carboxylic acid (0.30 g, 0.70 mmol), 1-(3-dimethylaminopropyl)-3-ethylcarbodiimide hydrochloride (0.19 g, 0.92 mmol) and N, N-diisopropylethylamine (0.18 mL, 1.02 mmol) in N,N-dimethylformamide (5 mL) was added 1-hydroxybenzotriazole (0.15 g, 1.10 mmol). The resulting mixture was stirred at ambient temperature for 10 minutes and 3,4-difluorobenzylamine (0.12 g, 0.85 mmol) was added. The reaction mixture was stirred at ambient temperature for 18 hours, diluted with ethyl acetate (30 mL) and washed with water and brine. The organic layer was dried over anhydrous sodium sulfate and filtered. The filtrate was concentrated in vacuo, and the residue was washed with ethyl acetate/hexanes (1/10) to afford N-(3,4-difluorobenzyl)-3-(1-(4-fluorobenzyl)-5-oxo-1H-1,2,4-triazol-4(5H)-yl)-1-(4-methoxybenzyl)-1H-pyrazole-5-carboxamide in 72% yield (0.28 g): mp 156-158° C.; ¹H NMR (300 MHz, CDCl₃) δ8.02 (s, 1H), 7.35-7.29 (m, 2H), 7.24-7.19 (m, 2H), 7.13-6.87 (m, 6H), 6.82-6.76 (m, 2H), 6.50 (t, J=6.0 Hz, 1H), 5.64 (s, 2H), 4.92 (s, 2H), 4.46 (d, J=6.0 Hz, 2H), 3.75 (s, 3H); MS (ES+) m/z 549.2 (M+1).

### PREPARATION 8

### Preparation of 3-(1-(4-Fluorobenzyl)-5-oxo-1H-1,2,4-triazol-4(5H)-yl)-1-(4-methoxybenzyl)-N-(pyridin-3-ylmethyl)-1H-pyrazole-5-carboxamide

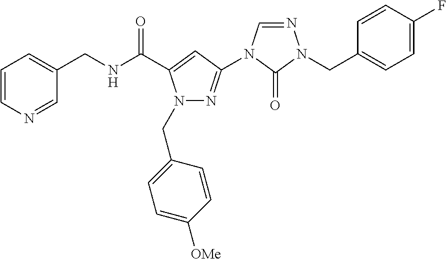 [SMILES: COc1ccc(Cn2nc(-n3cnn(Cc4ccc(F)cc4)c3=O)cc2C(=O)NCc2cccnc2)cc1]

To a solution of 3-(1-(4-fluorobenzyl)-5-oxo-1H-1,2,4-triazol-4(5H)-yl)-1-(4-methoxybenzyl)-1H-pyrazole-5-carboxylic acid (2.00 g, 4.72 mmol) in anhydrous tetrahdrofuran (110 mL) was added 1-hydroxy benzotriazole (HOBt) (1.28 g, 9.45 mmol), 2-(1H-benzotriazole-1-yl)-1,1,3,3-tetramethyluronium tetrafluoroborate (TBTU) (3.03 g, 9.45 mmol), N,N-diisopropylethylamine (3.66 g, 28.3 mmol) and pyridin-3-ylmethanamine (0.77 g, 7.09 mmol). The resulting solution was stirred at ambient temperature for 3 h and concentrated in vacuo. The residue was suspended in saturated aqueous sodium bicarbonate solution (400 mL). The solid was filtered, washed with water (3×100 mL) and dried under vacuum over night to afford 3-(1-(4-fluorobenzyl)-5-oxo-1H-1,2,4-triazol-4(5H)-yl)-1-(4-methoxybenzyl)-N-(pyridin-3-ylmethyl)-1H-pyrazole-5-carboxamide as a colorless solid (2.3 g, 95%): mp 210-211° C.; ¹H NMR (300 MHz, DMSO-d₆) δ 9.37 (t, J=5.9 Hz, 1H), 8.53-8.43 (m, 2H), 8.44 (s, 1H), 7.68-7.64 (m, 1H), 7.37-7.32 (m, 4H), 7.21-7.14 (m, 4H), 6.85-6.82 (m, 2H), 5.65 (s, 2H), 4.95 (s, 2H), 4.45 (d, J=5.9 Hz, 2H), 3.71 (s, 3H); MS (ES+) m/z 514.0 (M+1).

### PREPARATION 8.1

### Preparation of N-benzyl-3-(1-(4-fluorobenzyl)-5-oxo-1H-1,2,4-triazol-4(5H)-yl)-1-(4-methoxybenzyl)-1H-pyrazole-5-carboxamide

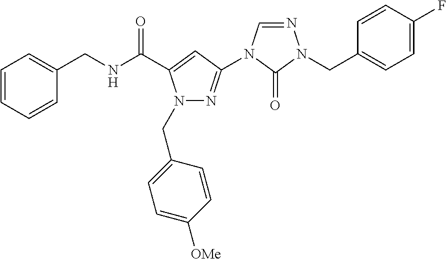 [SMILES: COc1ccc(Cn2nc(-n3cnn(Cc4ccc(F)cc4)c3=O)cc2C(=O)NCc2ccccc2)cc1]

Following the procedure as describe in PREPARATION 8, making variations as required to replace 3,4-difluorobenzylamine with benzylamine to react with 3-(1-4-fluorobenzyl)-5-oxo-1H-1,2,4-triazol-4(5H)-yl)-1-(4-methoxybenzyl)-1H-pyrazole-5-carboxylic acid, the title compound was obtained in 52% yield: ¹H NMR (300 MHz, CDCl₃) δ 8.02 (s, 1H), 7.36-7.18 (m, 9H), 7.04-6.95 (m, 3H), 6.83-6.76 (m, 2H), 6.32 (t, J=5.7 Hz, 1H), 5.65 (s, 2H), 4.92 (s, 2H), 4.54 (d, J=5.7 Hz, 2H), 3.76 (s, 3H); MS (ES+) m/z 513.3 (M+1).

### PREPARATION 8.2

### Preparation of 3-(1-(4-fluorobenzyl)-5-oxo-1H-1,2,4-triazol-4(5H)-yl)-1-(4-methoxybenzyl)-N-methyl-1H-pyrazole-5-carboxamide

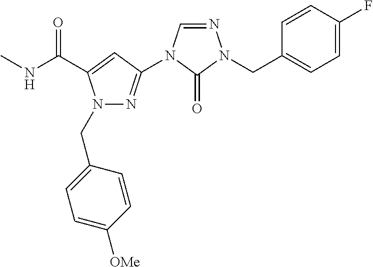 [SMILES: CNC(=O)c1cc(-n2cnn(Cc3ccc(F)cc3)c2=O)nn1Cc1ccc(OC)cc1]

Following the procedure as describe in PREPARATION 8, making variations as required to replace 3,4-difluorobenzylamine with methylamine to react with 3-(1-(4-fluorobenzyl)-5-oxo-1H-1,2,4-triazol-4(5H)-yl)-1-(4-methoxybenzyl)-1H-pyrazole-5-carboxylic acid, the title compound was obtained in 99% yield: MS (ES+) m/z 437.3 (M+1).

### EXAMPLE 1

### Synthesis of N-(3,4-difluorobenzyl)-3-(1-(4-fluorobenzyl)-5-oxo-1H-1,2,4-triazol-4(5H)-yl)-1H-pyrazole-5-carboxamide

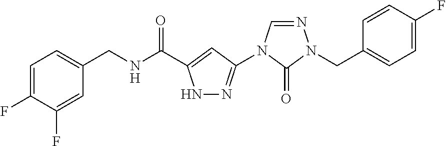 [SMILES: O=C(NCc1ccc(F)c(F)c1)c1cc(-n2cnn(Cc3ccc(F)cc3)c2=O)n[nH]1]

To a solution of N-(3,4-difluorobenzyl)-3-(1-(4-fluorobenzyl)-5-oxo-1H-1,2,4-triazol-4(5H)-yl)-1-(4-methoxybenzyl)-1H-pyrazole-5-carboxamide (0.28 g, 0.51 mmol) in dichloromethane (50 mL) and trifluoroacetic acid (5 mL) was added trifluoromethylsulfonic acid (0.3 mL, 3.37 mmol) at ambient temperature. The resulting reaction mixture was stirred at ambient temperature for 3 hours. The solvent was removed in vacuo, and the residue was neutralized to pH 4˜5 with saturated sodium bicarbonate. The resulting precipitate was filtered and washed with water and ethyl acetate to afford N-(3,4-difluorobenzyl)-3-(1-(4-fluorobenzyl)-5-oxo-1H-1,2,4-triazol-4(5H)-yl)-1H-pyrazole-5-carboxamide in 69% yield (0.15 g): mp 208-209° C.; ¹H NMR (300 MHz, DMSO-d₆) δ 9.13 (t, J=5.8 Hz, 1H), 8.39 (s, 1H), 7.43-7.27 (m, 4H), 7.25-7.04 (m, 4H), 4.92 (s, 2H), 4.40 (d, J=5.8 Hz, 2H); MS (ES+) ink 429.27 (M+1).

### EXAMPLE 2

### Synthesis of 3-(1-(4-fluorobenzyl)-5-oxo-1H-1,2,4-triazol-4(5H)-yl)-N-(pyridin-3-ylmethyl)-1H-pyrazole-3-carboxamide

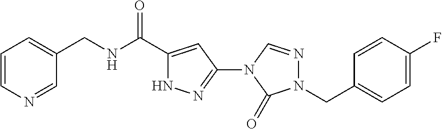 [SMILES: O=C(NCc1cccnc1)c1cc(-n2cnn(Cc3ccc(F)cc3)c2=O)n[nH]1]

To a mixture of 3-(1-(4-fluorobenzyl)-5-oxo-1H-1,2,4-triazol-4(5H)-yl)-1-(4-methoxybenzyl)-N-(pyridin-3-ylmethyl)-1H-pyrazole-5-carboxamide (3.50 g, 6.82 mmol) in dichloromethane (100 mL) was added trifluoroacetic acid (100 mL) and trifluoromethanesulfonic acid (5.30 g, 35.4 mmol). The dark purple solution was stirred at ambient temperature for 1.5 h and concentrated in vacuo. The red solid residue was suspended in a mixture of methanol (30 mL) and saturated aqueous sodium bicarbonate solution (500 mL) at 0° C. The white solid was filtered, washed with water (3×100 mL), ether (2×20 mL) and purified by column chromatography using 2-10% methanol in dichloromethane as an eluent to afford 5-(1-(4-fluorobenzyl)-5-oxo-1H-1,2,4-triazol-4(5H)-yl)-N-(pyridin-3-ylmethyl)-1H-pyrazole-3-carboxamide as an off-white solid (1.72 g, 64%): mp 235-236° C. (ethanol); ¹H NMR (300 MHz, DMSO-d₆) δ 13.84 (br, 1H), 9.31 (t, J=5.7 Hz, 1H), 8.56 (d, J=1.7 Hz, 1H), 8.48 (dd, J=4.7, 1.3 Hz, 1H), 8.45 (s, 1H), 7.78-7.68 (m, 1H), 7.39-7.31 (m, 4H), 7.21-7.16 (m, 2H), 4.96 (s, 2H), 4.49 (d, J=5.8 Hz, 2H); MS (ES+) m/z 394.1 (M+1).

### EXAMPLE 2.1

### Synthesis of N-benzyl-3-(1-(4-fluorobenzyl)-5-oxo-1H-1,2,4-triazol-4(5H)-yl)-1H-pyrazole-5-carboxamide

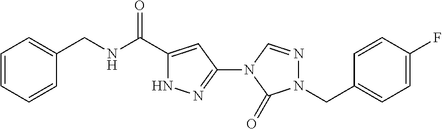 [SMILES: O=C(NCc1ccccc1)c1cc(-n2cnn(Cc3ccc(F)cc3)c2=O)n[nH]1]

Following the procedure as describe in EXAMPLE 2, making variations as required to replace N-(3,4-difluorobenzyl)-3-(1-(4-fluorobenzyl)-5-oxo-1H-1,2,4-triazol-4(5H)-yl)-1-(4-methoxybenzyl)-1H-pyrazole-5-carboxamide with N-benzyl-3-(1-(4-fluorobenzyl)-5-oxo-1H-1,2,4-triazol-4(5H)-yl)-1-(4-methoxybenzyl)-1H-pyrazole-5-carboxamide, the title compound was obtained in 99% yield: ¹H NMR (300 MHz, CDCl₃) δ 208-209° C.; ¹H NMR (300 MHz, DMSO-d₆) δ 9.01 (t, J=5.8 Hz, 1H), 8.37 (s, 1H), 7.48-7.02 (m, 10H), 4.91 (s, 2H), 4.42 (d, J=5.8 Hz, 2H); MS (ES+) m/z 393.3 (M+1).

### EXAMPLE 2.2

### Synthesis of 3-(1-(4-Fluorobenzyl)-5-oxo-1H-1,2,4-triazol-4(5H)-yl)-N-methyl-1H-pyrazole-5-carboxamide

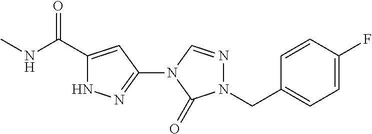 [SMILES: CNC(=O)c1cc(-n2cnn(Cc3ccc(F)cc3)c2=O)n[nH]1]

Following the procedure as describe in EXAMPLE 2, making variations as required to replace N-(3,4-difluorobenzyl)-3-(1-(4-fluorobenzyl)-5-oxo-1H-1,2,4-triazol-4(5H)-yl)-1-(4-methoxybenzyl)-1H-pyrazole-5-carboxamide with 3-(1-(4-fluorobenzyl)-5-oxo-1H-1,2,4-triazol-4(5H)-yl)-1-(4-methoxybenzyl)-N-methyl-1H-pyrazole-5-carboxamide, the title compound was obtained in 29% yield: mp 268-269° C.; ¹H NMR (300 MHz, DMSO-d₆) δ 13.72 (s, 1H), 8.63 (t, J=4.6 Hz, 1H), 8.40 (s, 1H), 7.36-7.28 (m, 2H), 7.21-7.10 (m, 3H), 4.92 (s, 2H), 2.73 (d, J=4.6 Hz, 3H); MS (ES+) m/z 317.2 (M+1).

### EXAMPLE 3

### Synthesis of 3-(3-(4-fluorobenzyl)-2-oxoimidazolidin-1-yl)-N-(pyridin-2-ylmethyl)-1H-pyrazole-5-carboxamide

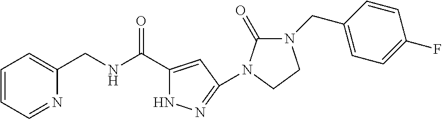 [SMILES: O=C(NCc1ccccn1)c1cc(N2CCN(Cc3ccc(F)cc3)C2=O)n[nH]1]

To a solution of 3-(3-(4-fluorobenzyl)-2-oxoimidazolidin-1-yl)-1-(4-methoxybenzyl)-1H-pyrazole-5-carboxylic acid (0.12 g, 0.28 mmol) in anhydrous tetrahdrofuran (5 mL) was added 1-hydroxybenzotriazole (0.077 g, 0.57 mmol), 2-(1H-benzotriazole-1-yl)-1,1,3,3-tetramethyluronium tetrafluoroborate (0.18 g, 0.57 mmol), N,N-diisopropylethylamine (0.22 g, 1.80 mmol) and pyridin-2-ylmethanamine (0.46 g, 0.43 mmol). The resulting mixture was stirred at ambient temperature for 72 h, and concentrated in vacuo. The residue was suspended in saturated aqueous sodium bicarbonate solution (15 mL). The solid was filtered, washed with water (3×10 mL) and dried in vacuo. This solid was then suspended in dichloromethane (3 mL), followed by the addition of trifluoroacetic acid (2 mL) and trifluoromethanesulfonic acid (0.13 mL). The dark purple solution was stirred at ambient temperature for 2 h and concentrated in vacuo. The red solid residue was suspended in saturated aqueous sodium bicarbonate solution (15 mL) at 0° C. and the resulting white solid was filtered, washed with water (3 x 10 mL), ether (2×10 mL) and then recrystallized from N,N-dimethylformamide/water to afford 3-(3-(4-fluorobenzyl)-2-oxoimidazolidin-1-yl)-N-(pyridin-2-ylmethyl)-1H-pyrazole-5-carboxamide as a white solid in 63% yield (0.07 g): mp 235-237° C. (N,N-dimethylformamide/water); ¹H NMR (300 MHz, DMSO-d₆) δ 13.03 (d, J=1.9 Hz, 1H), 9.19 (t, J=5.9 Hz, 1H), 8.52-8.50 (m, 1H), 7.79-7.74 (m, 1H), 7.37-7.16 (m, 7H), 4.52 (d, J=5.9 Hz, 2H), 4.37 (s, 2H), 3.81-3.75 (m, 2H), 3.38-3.34 (m, 2H); MS (ES+) m/z 395.0 (M+1).

### EXAMPLE 4

### Synthesis of 3-(3-(4-fluorobenzyl)-2-oxoimidazolidin-1-yl)-N-((5-methyl-1H-pyrazol-3-yl)methyl)-1H-pyrazole-5-carboxamide

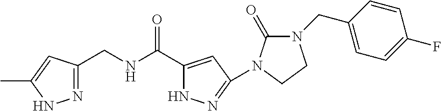 [SMILES: Cc1cc(CNC(=O)c2cc(N3CCN(Cc4ccc(F)cc4)C3=O)n[nH]2)n[nH]1]

To a solution of 3-(3-(4-fluorobenzyl)-2-oxoimidazolidin-1-yl)-1-(4-methoxybenzyl)-1H-pyrazole-5-carboxylic acid (0.12 g, 0.28 mmol) in anhydrous tetrahdrofuran (5 mL) was added 1-hydroxybenzotriazole (0.077 g, 0.57 mmol), 2-(1H-benzotriazole-1-yl)-1,1,3,3-tetramethyluronium tetrafluoroborate (0.18 g, 0.57 mmol), N, N-diisopropylethylamine (0.22 g, 1.80 mmol) and (5-methyl-1H-pyrazol-3-yl)methanamine hydrochloride (0.066 g, 0.43 mmol). The resulting solution was stirred at ambient temperature for 72 h and concentrated in vacuo. The residue was suspended in saturated aqueous sodium bicarbonate solution (15 mL). The solid was collected by filtration, washed with water (3×10 mL) and dried under vacuum. This dried solid was then suspended in dichloromethane (3 mL), followed by the addition of trifluoroacetic acid (2 mL) and trifluoromethanesulfonic acid (0.13 mL). The dark purple solution was stirred at ambient temperature for 2 h and concentrated in vacuo. The red solid residue was suspended in saturated aqueous sodium bicarbonate solution (15 mL) at 0° C. The white solid was collected by filtration, washed with water (3×10 mL), ether (2×10 mL) and then recrystallized from N,N-dimethylformamide/water to afford 3-(3-(4-fluorobenzyl)-2-oxoimidazolidin-1-yl)-N-((5-methyl-1H-pyrazol-3-yl)methyl)-1H-pyrazole-5-carboxamide as a white solid (0.032 g, 29%): mp 254-255° C. (N,N-dimethylformamide/water); ¹H NMR (300 MHz, DMSO-d₆) δ 12.95 (br s, 1H), 12.27 (br s, 1H), 8.93 (br s, 1H), 7.36-7.31 (m, 2H), 7.21-7.15 (m, 3H), 5.90 (s, 1H), 4.36 (s, 2H), 4.33 (d, J=5.8 Hz, 2H), 3.80-3.75 (m, 2H), 3.38-3.32 (m, 2H), 2.16 (s, 3H); MS (ES+) m/z 397.8 (M+1).

### EXAMPLE 5

### Synthesis of 3-(3-(4-fluorobenzyl)-2-oxoimidazolidin-1-yl)-N-((6-methylisoxazol-3-yl)methyl)-1H-pyrazole-5-carboxamide

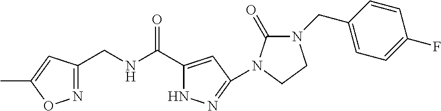 [SMILES: Cc1cc(CNC(=O)c2cc(N3CCN(Cc4ccc(F)cc4)C3=O)n[nH]2)no1]

To a solution of 3-(3-(4-fluorobenzyl)-2-oxoimidazolidin-1-yl)-1-(4-methoxybenzyl)-1H-pyrazole-5-carboxylic acid (0.12 g, 0.28 mmol) in anhydrous tetrahdrofuran (5 mL) was added 1-hydroxy benzotriazole (0.077 g, 0.57 mmol), 2-(1H-benzotriazole-1-yl)-1,1,3,3-tetramethyluronium tetrafluoroborate (0.18 g, 0.57 mmol), N,N-diisopropylethylamine (0.22 g, 1.80 mmol) and (5-methylisoxazol-3-yl)methanamine hydrochloride (0.063 g, 0.43 mmol). The resulting solution was stirred at ambient temperature for 72 h and concentrated in vacuo. The residue was suspended in saturated aqueous sodium bicarbonate solution (15 mL). The solid was collected by filtration, washed with water (3×10 mL) and dried under vacuum. This dried solid was then suspended in dichloromethane (3 mL), followed by the addition of trifluoroacetic acid (2 mL) and trifluoromethanesulfonic acid (0.13 mL). The dark purple solution was stirred at ambient temperature for 2 h and concentrated in vacuo. The red solid residue was suspended in saturated aqueous sodium bicarbonate solution (15 mL) at 0° C. The white solid was collected by filtration, washed with water (3×10 mL), ether (2×10 mL) and recrystallized from N,N-dimethylformamide/water to afford 3-(3-(4-fluorobenzyl)-2-oxoimidazolidin-1-yl)-N-((5-methylisoxazol-3-yl)methyl)-1H-pyrazole-5-carboxamide as an off-white solid (0.069 g, 62%): mp 294-296° C. (dec.) (N,N-dimethylformamide/water); ¹H NMR (300 MHz, DMSO-d₆) δ 13.04 (d, J=1.7 Hz, 1H), 9.14 (t, J=5.9 Hz, 1H), 7.36-7.31 (m, 2H), 7.21-7.16 (m, 3H), 6.15 (s, 1H), 4.41 (d, J=5.9 Hz, 2H), 4.37 (s, 2H), 3.81-3.75 (m, 2H), 3.38-3.34 (m, 2H), 2.37 (s, 3H); MS (ES+) ink 398.8 (M+1).

### EXAMPLE 6

### Synthesis of 3-(3-(4-fluorobenzyl)-2-oxoimidazolidin-1-yl)-N-(pyridin-3-ylmethyl)-1H-pyrazole-5-carboxamide

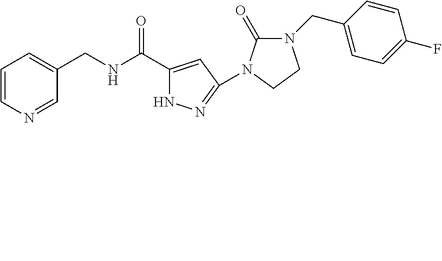 [SMILES: O=C(NCc1cccnc1)c1cc(N2CCN(Cc3ccc(F)cc3)C2=O)n[nH]1]

To a solution of 3-(3-(4-fluorobenzyl)-2-oxoimidazolidin-1-yl)-1-(4-methoxybenzyl)-1H-pyrazole-5-carboxylic acid (0.15 g, 0.35 mmol) in anhydrous tetrahdrofuran (6 mL) was added 1-hydroxybenzotriazole (0.096 g, 0.71 mmol), 2-(1H-benzotriazole-1-yl)-1,1,3,3-tetramethyluronium tetrafluoroborate (0.23 g, 0.71 mmol) and N,N-diisopropylethylamine (0.27 g, 2.12 mmol). The mixture was stirred for 10 minutes, followed by the addition of pyridin-3-ylmethanamine (0.057 g, 0.53 mmol). The resulting solution was stirred at ambient temperature for 2 h and concentrated in vacuo. The residue was suspended in saturated aqueous sodium bicarbonate solution (15 mL). The solid was collected by filtration, washed with water (3×10 mL) and dried under vacuum. This dried solid was then suspended in dichloromethane (3 mL), followed by the addition of trifluoroacetic acid (2 mL) and trifluoromethanesulfonic acid (0.15 mL). The dark purple solution was stirred at ambient temperature for 2 h and concentrated in vacuo. The red solid residue was suspended in saturated aqueous sodium bicarbonate solution (15 mL) at 0° C. The white solid was collected by filtration, washed with water (3×10 mL) and ether (2×10 mL). The solid was purified by column chromatography using 2-10% methanol in dichloromethane as eluent to afford 3-(3-(4-fluorobenzyl)-2-oxoimidazolidin-1-yl)-N-(pyridin-3-ylmethyl)-1H-pyrazole-5-carboxamide as a white solid (0.09 g, 65%): mp 251-253° C.; ¹H NMR (300 MHz, DMSO-d₆) δ 13.03 (s, 1H), 9.16 (t, J=5.8 Hz, 1H), 8.54-8.46 (m, 2H), 7.72-7.70 (m, 1H), 7.39-7.31 (m, 3H), 7.21-7.15 (m, 3H), 4.45 (d, J=5.8 Hz, 2H), 4.37 (s, 2H), 3.81-3.75 (m, 2H), 3.38-3.32 (m, 2H); MS (ES+) m/z 395.1 (M+1).

### EXAMPLE 7

### Synthesis of 3-(1-(4-fluorobenzyl)-5-oxo-1H-1,2,4-triazol-4(5H)-yl)-N-((5-methylisoxazol-3-yl)methyl)-1H-pyrazole-5-carboxamide

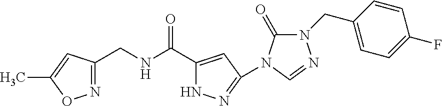 [SMILES: Cc1cc(CNC(=O)c2cc(-n3cnn(Cc4ccc(F)cc4)c3=O)n[nH]2)no1]

To a solution of 3-(3-(4-fluorobenzyl)-2-oxoimidazolidin-1-yl)-1-(4-methoxybenzyl)-1H-pyrazole-5-carboxylic acid (0.12 g, 0.28 mmol) in anhydrous tetrahdrofuran (5 mL) was added 1-hydroxybenzotriazole (0.077 g, 0.57 mmol), 2-(1H-benzotriazole-1-yl)-1,1,3,3-tetramethyluronium tetrafluoroborate (0.18 g, 0.57 mmol), N,N-diisopropylethylamine (0.22 g, 1.80 mmol) and (5-methylisoxazol-3-yl)methanamine hydrochloride (0.063 g, 0.43 mmol). The resulting solution was stirred at ambient temperature for 72 h and concentrated in vacuo. The residue was suspended in saturated aqueous sodium bicarbonate solution (15 mL). The solid was collected by filtration, washed with water (3×10 mL) and dried under vacuum. This dried solid was then suspended in dichloromethane (3 mL), followed by the addition of trifluoroacetic acid (3 mL) and trifluoromethanesulfonic acid (0.13 mL). The dark purple solution was stirred at ambient temperature for 2 h and concentrated in vacuo. The red solid residue was suspended in saturated aqueous sodium bicarbonate solution (15 mL) at 0° C. The white solid was collected by filtration, washed with water (3×10 mL), ether (2×10 mL) and recrystallized from N,N-dimethylformamide/water to afford 3-(1-(4-fluorobenzyl)-5-oxo-1H-1,2,4-triazol-4(5H)-yl)-N-((5-methylisoxazol-3-yl)methyl)-1H-pyrazole-5-carboxamide as an off-white solid (0.09 g, 81%): mp 276-277° C. (N,N-dimethylformamide/water); ¹H NMR (300 MHz, DMSO-d₆) δ 13.85 (s, 1H), 9.31 (t, J=5.5 Hz, 1H), 8.45 (s, 1H), 7.38-7.31 (m, 3H), 7.22-7.16 (m, 2H), 6.19-6.15 (m, 1H), 4.96 (s, 2H), 4.45 (d, J=5.9 Hz, 2H), 2.37 (s, 3H); MS (ES+) m/z 397.8 (M+1).

### EXAMPLE 8

### Synthesis of 3-(1-(4-fluorobenzyl)-5-oxo-1H-1,2,4-triazol-4(5H)-yl)-N-(pyridin-2-ylmethyl)-1H-pyrazole-5-carboxamide

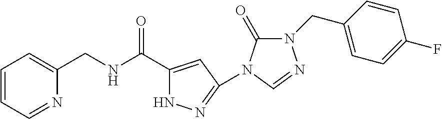 [SMILES: O=C(NCc1ccccn1)c1cc(-n2cnn(Cc3ccc(F)cc3)c2=O)n[nH]1]

To a solution of 3-(3-(4-fluorobenzyl)-2-oxoimidazolidin-1-yl)-1-(4-methoxybenzyl)-1H-pyrazole-5-carboxylic acid (0.12 g, 0.28 mmol) in anhydrous tetrahdrofuran (5 mL) was added 1-hydroxybenzotriazole (0.077 g, 0.57 mmol), 2-(1H-benzotriazole-1-yl)-1,1,3,3-tetramethyluronium tetrafluoroborate (0.18 g, 0.57 mmol), N,N-diisopropylethylamine (0.22 g, 1.80 mmol) and pyridin-2-ylmethanamine (0.046 g, 0.43 mmol). The resulting solution was stirred at ambient temperature for 72 h and concentrated in vacuo. The residue was suspended in saturated aqueous sodium bicarbonate solution (15 mL). The solid was collected by filtration, washed with water (3×10 mL) and dried under vacuum. This dried solid was then suspended in dichloromethane (3 mL), followed by the addition of trifluoroacetic acid (3 mL) and trifluoromethanesulfonic acid (0.13 mL). The dark purple solution was stirred at ambient temperature for 2 h and it was concentrated in vacuo. The red solid residue was suspended in saturated aqueous sodium bicarbonate solution (15 mL) at 0° C. The white solid was collected by filtration, washed with water (3×10 mL), ether (2×10 mL) and recrystallized from N,N-dimethylformamide/water to afford 3-(1-(4-fluorobenzyl)-5-oxo-1H-1,2,4-triazol-4(5H)-yl)-N-(pyridin-2-ylmethyl)-1H-pyrazole-5-carboxamide as an off-white solid (0.087 g, 79%): mp 224-225° C. (N,N-dimethylformamide/water); ¹H NMR (300 MHz, DMSO-d₆) δ 13.83 (br, 1H), 9.36 (t, J=5.9 Hz, 1H), 8.52 (d, J=4.2 Hz, 1H), 8.46 (s, 1H), 7.80-7.74 (m, 1H), 7.38-7.26 (m, 5H), 7.22-7.16 (m, 2H), 4.97 (s, 2H), 4.56 (d, J=5.8 Hz, 2H); MS (ES+) m/z 393.8 (M+1).

### EXAMPLE 8.1

### Synthesis of 3-(3-(4-fluorobenzyl)-2-oxoimidazolidin-1-yl)-N-((1-methyl-1H-pyrazol-4-yl)methyl)-1H-pyrazole-5-carboxamide

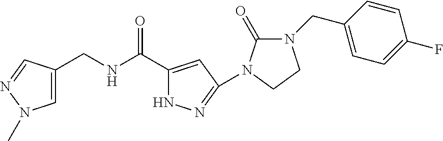 [SMILES: Cn1cc(CNC(=O)c2cc(N3CCN(Cc4ccc(F)cc4)C3=O)n[nH]2)cn1]

Following the procedure as described in EXAMPLE 8, making variations as required to replace pyridin-2-ylmethanamine with (1-methyl-1H-pyrazol-3-yl)methanamine hydrochloride to react with 3-(3-(4-fluorobenzyl)-2-oxoimidazolidin-1-yl)-1-(4-methoxybenzyl)-1H-pyrazole-5-carboxylic acid, the title compound was obtained as an off-white solid in 49% yield: mp 244-245° C. (N,N-dimethylformamide/water); ¹H NMR (300 MHz, DMSO-d₆) δ 12.95 (br s, 1H), 8.89 (1, J=5.7 Hz, 1H), 7.58 (s, 1H), 7.36-7.31 (m, 3H), 7.21-7.13 (m, 3H), 4.36 (s, 2H), 4.24 (d, J=5.7 Hz, 2H), 3.80-3.74 (m, 5H), 3.37-3.32 (m, 21-1); MS (ES+) m/z 397.8 (M+1).

### EXAMPLE 8.2

### Synthesis of 3-(3-(4-fluorobenzyl)-2-oxoimidazolidin-1-yl)-N-(thiazol-2-ylmethyl)-1H-pyrazole-5-carboxamide

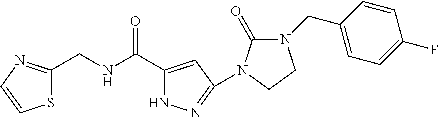 [SMILES: O=C(NCc1nccs1)c1cc(N2CCN(Cc3ccc(F)cc3)C2=O)n[nH]1]

Following the procedure as described in EXAMPLE 8, making variations as required to replace pyridin-2-ylmethanamine with thiazol-2-ylmethanamine hydrochloride to react with 3-(3-(4-fluorobenzyl)-2-oxoimidazolidin-1-yl)-1-(4-methoxybenzyl)-1H-pyrazole-5-carboxylic acid, the title compound was obtained as a white solid in 76% yield: mp 272-274° C. (N,N-dimethylformamide/water); ¹H NMR (300 MHz, DMSO-d₆) δ 13.11 (d, J=1.7 Hz, 1H), 9.47 (t, J=6.0 Hz, 1H), 7.74 (d, J=3.3 Hz, 1H), 7.64 (d, J=3.3 Hz, 1H), 7.37-7.32 (m, 2H), 7.23-7.16 (m, 3H), 4.71 (d, J=6.0 Hz, 2H), 4.37 (s, 2H), 3.82-3/6 (m, 2H), 3.38-3.34 (m, 2H); MS (ES+) m/z 400.9 (M+1).

### EXAMPLE 8.3

### Synthesis of N-benzyl-3-(3-(4-fluorobenzyl)-2-oxoimidazolidin-1-yl)-1H-pyrazole-5-carboxamide

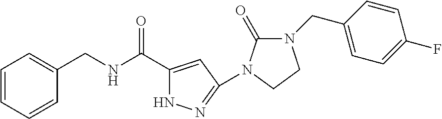 [SMILES: O=C(NCc1ccccc1)c1cc(N2CCN(Cc3ccc(F)cc3)C2=O)n[nH]1]

Following the procedure as described in EXAMPLE 8, making variations as required to replace pyridin-2-ylmethanamine with benzylamine to react with 3-(3-(4-fluorobenzyl)-2-oxoimidazolidin-1-yl)-1-(4-methoxybenzyl)-1H-pyrazole-5-carboxylic acid, the title compound was obtained as a white solid in 66% yield: mp 262-263° C. (N,N-dimethylformamide/water); ¹H NMR (300 MHz, DMSO-d₆) δ 13.00 (d, J=1.8 Hz, 1H), 9.12 (t, J=6.0 Hz, 1H), 7.36-7.15 (m, 10H), 4.43 (d, J=6.0 Hz, 2H), 4.37 (s, 2H), 7.81-3.75 (m, 2H), 3.38-3.32 (m, 2H); MS (ES+) m/z 399.8 (M+1).

### EXAMPLE 8.4

### Synthesis of 3-(3-(4-fluorobenzyl)-2-oxoimidazolidin-1-yl)-1H-pyrazole-5-carboxamide

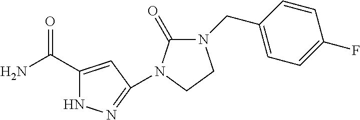 [SMILES: NC(=O)c1cc(N2CCN(Cc3ccc(F)cc3)C2=O)n[nH]1]

Following the procedure as described in EXAMPLE 8, making variations as required to replace pyridin-2-ylmethanamine with ammonium chloride to react with 3-(3-(4-fluorobenzyl)-2-oxoimidazolidin-1-yl)-1-(4-methoxybenzyl)-1H-pyrazole-5-carboxylic acid, the title compound was obtained as an off-white solid in 61% yield: mp 256-258° C. (N,N-dimethylformamide/water); ¹H NMR (300 MHz, DMSO-d₆) δ 12.91 (d, J=1.6 Hz, 1H), 7.98 (s, 1H), 7.44 (s, 1H), 7.36-7.31 (m, 2H), 7.21-7.16 (m, 2H), 7.11 (d, J=2.2 Hz, 1 H), 4.37 (s, 2H), 3.80-3.75 (m, 2H), 3.37-3.32 (m, 2H); MS (ES+) m/z 303.8 (M+1).

### EXAMPLE 8.5

### Synthesis of 3-(1-(4-fluorobenzyl)-5-oxo-1H-1,2,4-triazol-4(5H)-yl)-1H-pyrazole-5-carboxamide

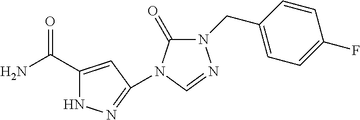 [SMILES: NC(=O)c1cc(-n2cnn(Cc3ccc(F)cc3)c2=O)n[nH]1]

Following the procedure as described in EXAMPLE 8, making variations as required to replace pyridin-2-ylmethanamine with ammonium chloride to react with 3-(1-(4-fluorobenzyl)-5-oxo-1H-1,2,4-triazol-4(5H)-yl)-1-(4-methoxybenzyl)-1H-pyrazole-5-carboxylic acid, the title compound was obtained as a white solid in 40% yield: mp>270° C. (N,N-dimethylformamide/water); ¹H NMR (300 MHz, DMSO-d₆) δ 13.73 (s, 1H), 8.43 (s, 1H), 8.14 (s, 1H), 7.63 (s, 1H), 7.38-7.33 (m, 2H), 7.24-7.16 (m, 3H), 4.96 (s, 2H); MS (ES+) m/z 302.9 (M+1).

### EXAMPLE 8.6

### Synthesis of 3-(1-(4-fluorobenzyl)-5-oxo-1H-1,2,4-triazol-4(5H)-yl)-N-(oxazol-4-ylmethyl)-1H-pyrazole-5-carboxamide

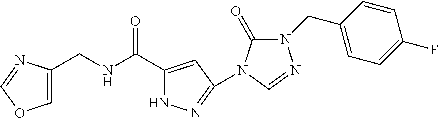 [SMILES: O=C(NCc1cocn1)c1cc(-n2cnn(Cc3ccc(F)cc3)c2=O)n[nH]1]

Following the procedure as described in EXAMPLE 8, making variations as required to replace pyridin-2-ylmethanamine with oxazol-4-ylmethanamine hydrochloride to react with 3-(1-(4-fluorobenzyl)-5-oxo-1H-1,2,4-triazol-4(5H)-yl-1-(4-methoxybenzyl)-1H-pyrazole-5-carboxylic acid, the title compound was obtained as a white solid in 66% yield: mp 267-268° C. (N,N-dimethylformamide/water); ¹H NMR (300 MHz, DMSO-d₆) δ 13.80 (s, 1H), 9.18 (t, J=5.6 Hz, 1H), 8.44 (s, 1H), 8.34 (s, 1H), 8.00 (s, 1H), 7.38-7.32 (m, 3H), 7.22-7.16 (m, 2H), 4.96 (s, 2H), 4.36 (d, J=5.6 Hz, 2H); MS (ES+) m/z 383.8 (M+1).

### EXAMPLE 8.7

### Synthesis of 3-(1-(4-fluorobenzyl)-5-oxo-1H-1,2,4-triazol-4(5H)-yl)-N-((3-methyl-1H-pyrazol-5-yl)methyl)-1H-pyrazole-5-carboxamide

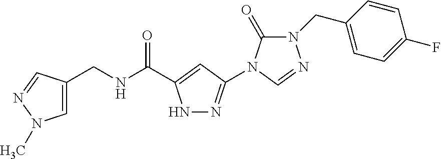 [SMILES: Cn1cc(CNC(=O)c2cc(-n3cnn(Cc4ccc(F)cc4)c3=O)n[nH]2)cn1]

Following the procedure as described in EXAMPLE 8, making variations as required to replace pyridin-2-ylmethanamine with (1-methyl-1H-pyrazol-4-yl)methanamine to react with 3-(1-(4-fluorobenzyl)-5-oxo-1H-1,2,4-triazol-4(5H)-yl)-1-(4-methoxybenzyl)-1H-pyrazole-5-carboxylic acid, the title compound was obtained as a white solid in 53% yield: mp 239-241° C. (N,N-dimethylformamide/water); ¹H NMR (300 MHz, DMSO-d₆) δ 13.77 (s, 1H), 9.04 (t, J=5.6 Hz, 1H), 8.44 (s, 1H), 7.61 (s, 1H), 7.37-7.27 (m, 4H), 7.22-7.16 (m, 2H), 4.95 (s, 2H), 4.27 (d, J=5.6 Hz, 2H), 3.78 (s, 3H); MS (ES+) m/z 396.9 (M+1).

### EXAMPLE 8.8

### Synthesis of 3-(1-(4-fluorobenzyl)-5-oxo-1H-1,2,4-triazol-4(5H)-yl)-N-(pyridin-4-ylmethyl)-1H-pyrazole-5-carboxamide

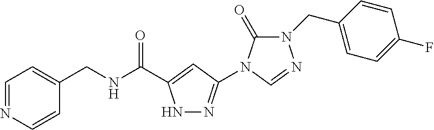 [SMILES: O=C(NCc1ccncc1)c1cc(-n2cnn(Cc3ccc(F)cc3)c2=O)n[nH]1]

Following the procedure as described in EXAMPLE 8, making variations as required to replace pyridin-2-ylmethanamine with pyridin-4-ylmethanamine to react with 3-(1-(4-fluorobenzyl)-5-oxo-1H-1,2,4-triazol-4(5H)-yl)-1-(4-methoxybenzyl)-1H-pyrazole-5-carboxylic acid, the title compound was obtained as a white solid in 77% yield: mp 262-263° C. (N,N-dimethylformamide/water); ¹H NMR (300 MHz, DMSO-d₆) δ 13.86 (br s, 1H), 9.36 (t, J=5.8 Hz, 1H), 8.53-8.51 (m, 2H), 8.46 (s, 1H), 7.38-7.30 (m, 5H), 7.22-7.16 (m, 2H), 4.97 (s, 2H), 4.49 (d, J=5.8 Hz, 2H); MS (ES+) m/z 393.8 (M+1).

### EXAMPLE 9

### Measuring Stearoyl-CoA Desaturase Inhibition Activity of a Test Compound Using Mouse Liver Microsomes

The identification of compounds of the invention as SCD inhibitors was readily accomplished using the SCD microsomal assay procedure described in Shanklin J. and Summerville C., Proc. Natl. Acad. Sci. USA (1991), Vol. 88, pp. 2510-2514.

### Preparation of Mouse Liver Microsomes:

Male ICR outbread mice, on a high-carbohydrate, low fat diet, under light halothane (15% in mineral oil) anesthesia are sacrificed by exsanguination during periods of high enzyme activity. Livers are immediately rinsed with cold 0.9% NaCl solution, weighed and minced with scissors. All procedures are performed at 4° C. unless specified otherwise. Livers are homogenized in a solution (1/3 w/v) containing 0.25 M sucrose, 62 mM potassium phosphate buffer (pH 7.0), 0.15 M KCl, 15 mM N-acetyleysteine, 5 mM MgCl₂, and 0.1 mM EDTA using 4 strokes of a Potter-Elvehjem tissue homogenizer. The homogenate is centrifuged at 10,400×g for 20 min to eliminate mitochondria and cellular debris. The supernatant is filtered through a 3-layer cheesecloth and centrifuged at 105,000×g for 60 min. The microsomal pellet is gently resuspended in the same homogenization solution with a small glass/teflon homogenizer and stored at ˜70° C. The absence of mitochondrial contamination is enzymatically assessed. The protein concentration is measured using bovine serum albumin as the standard.

Incubation of Mouse Liver Microsomes with Test Compounds:

Desaturase activity is measured as the release of ³H₂O from [9,10-³H]stearoyl-CoA. Reactions per assay point conditions are as follows: 2 μL 1.5 mM stearoyl-CoA, 0.25 μL 1 mCi/mL ³H stearoyl CoA, 10 μL 20 mM NADH, 36.75 μL 0.1 M PK buffer (K₂HPO₄/NaH₂PO₄, pH 7.2). The test compound or control solution is added in a 1 μL volume. Reactions are started by adding 50 μL of microsomes (1.25 mg/mL). The plates are mixed and after 15 min incubation on a heating block (25° C.), the reactions are stopped by the addition of 10 μL 60% PCA. An aliquot of 100 μL is then transferred to a filter plate pretreated with charcoal and the plate centrifuged at 4000 rpm for 1 minute. The flow through containing the ³H₂O released by the SCD1 desaturation reaction is added to scintillation fluid and the radioactivity measured in a Packard TopCount. The data is analysed to identify the IC₅₀ for test compounds and reference compounds.

Representative compounds of the invention showed activity as inhibitors of SCD when tested in this assay. The activity was defined in terms of % SCD enzyme activity remaining at the desired concentration of the test compound or as the IC₅₀ concentration. The IC₅₀ (affinity) of the example compounds toward the stearoyl-CoA desaturase is comprised between around 20 mM and 0.0001 μM or between around 5 μM and 0.0001 μM or between around 1 μM and 0.0001 μM.

The following Table provides data that exemplifies representative compounds and their Microsomal IC₅₀ (μM) data.

| Example | Compound name | Microsomal IC₅₀ (μM) |
|---|---|---|
| 1 | N-(3,4-difluorobenzyl)-3-(1-(4-fluorobenzyl)-5-oxo- | 0.008 |
| | 1H-1,2,4-triazol-4(5H)-yl)-1H-pyrazole-5- | |
| | carboxamide | |
| 2 | 3-(1-(4-fluorobenzyl)-5-oxo-1H-1,2,4-triazol-4(5H)- | 0.007 |
| | yl)-N-(pyridin-3-ylmethyl)-1H-pyrazole-3- | |
| | carboxamide | |
| 2.1 | N-Benzyl-3-(1-(4-fluorobenzyl)-5-oxo-1H-1,2,4- | 0.006 |
| | triazol-4(5H)-yl)-1H-pyrazole-5-carboxamide | |
| 2.2 | 3-(1-(4-Fluorobenzyl)-5-oxo-1H-1,2,4-triazol-4(5H)- | 0.385 |
| | yl)-N-methyl-1H-pyrazole-5-carboxamide | |
| 3 | 3-(3-(4-fluorobenzyl)-2-oxoimidazolidin-1-yl)-N- | 0.045 |
| | (pyridin-2-ylmethyl)-1H-pyrazole-5-carboxamide | |
| 4 | 3-(3-(4-fluorobenzyl)-2-oxoimidazolidin-1-yl)-N-((5- | 0.088 |
| | methyl-1H-pyrazol-3-yl)methyl)-1H-pyrazole-5- | |
| | carboxamide | |
| 5 | 3-(3-(4-fluorobenzyl)-2-oxoimidazolidin-1-yl)-N-((5- | 0.037 |
| | methylisoxazol-3-yl)methyl)-1H-pyrazole-5- | |
| | carboxamide | |
| 6 | 3-(3-(4-fluorobenzyl)-2-oxoimidazolidin-1-yl)-N- | 0.003 |
| | (pyridin-3-ylmethyl)-1H-pyrazole-5-carboxamide | |
| 7 | 3-(1-(4-fluorobenzyl)-5-oxo-1H-1,2,4-triazol-4(5H)- | 0.022 |
| | yl)-N-((5-methylisoxazol-3-yl)methyl)-1H-pyrazole- | |
| | 5-carboxamide | |
| 8 | 3-(1-(4-fluorobenzyl)-5-oxo-1H-1,2,4-triazol-4(5H)- | 0.023 |
| | yl)-N-(pyridin-2-ylmethyl)-1H-pyrazole-5- | |
| | carboxamide | |
| 8.1 | 3-(3-(4-fluorobenzyl)-2-oxoimidazolidin-1-yl)-N-((1- | 0.122 |
| | methyl-1H-pyrazol-4-yl)methyl)-1H-pyrazole-5- | |
| | carboxamide | |
| 8.2 | 3-(3-(4-fluorobenzyl)-2-oxoimidazolidin-1-yl)-N- | 0.008 |
| | (thiazol-2-ylmethyl)-1H-pyrazole-5-carboxamide | |
| 8.3 | N-benzyl-3-(3-(4-fluorobenzyl)-2-oxoimidazolidin-1- | 0.011 |
| | yl)-1H-pyrazole-5-carboxamide | |
| 8.4 | 3-(3-(4-fluorobenzyl)-2-oxoimidazolidin-1-yl)-1H- | 0.368 |
| | pyrazole-5-carboxamide | |
| 8.5 | 3-(1-(4-fluorobenzyl)-5-oxo-1H-1,2,4-triazol-4(5H)- | 0.488 |
| | yl)-1H-pyrazole-5-carboxamide | |
| 8.6 | 3-(1-(4-fluorobenzyl)-5-oxo-1H-1,2,4-triazol-4(5H)- | 0.165 |
| | yl)-N-(oxazol-4-ylmethyl)-1H-pyrazole-5- | |
| | carboxamide | |
| 8.7 | 3-(1-(4-fluorobenzyl)-5-oxo-1H-1,2,4-triazol-4(5H)- | 0.147 |
| | yl)-N-((3-methyl-1H-pyrazol-5-yl)methyl)-1H- | |
| | pyrazole-5-carboxamide | |
| 8.8 | 3-(1-(4-fluorobenzyl)-5-oxo-1H-1,2,4-triazol-4(5H)- | 0.065 |
| | yl)-N-(pyridin-4-ylmethyl)-1H-pyrazole-5- | |
| | carboxamide | |

Those skilled in the art are aware of a variety of modifications to this assay that can be useful for measuring inhibition of stearoyl-CoA desaturase activity in microsomes or in cells by test compounds.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound of Formula (I): 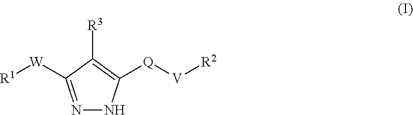
wherein,
Q is 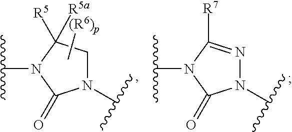
W is —N(R)C(O)—, —C(O)N(R⁸)—, C₁-C₆alkylene, C₂-C₆alkeneylene, C₂-C₆alkynylene or a direct bond;
V is selected from a C₁-C₆alkylene;
n is 1, 2, or 3;
p is 0, 1, 2, 3, 4, 5, or 6;
R¹ is hydrogen, an optionally substituted C₁-C₇alkyl, C₂-C₆alkenyl, C₂-C₆alkynyl, C₁-C₇alkoxy, hydroxyC₁-C₄alkyl, C₁-C₇alkoxyC₁-C₄alkyl, an optionally substituted C₃-C₇cycloalkyl, an optionally substituted C₃-C₇cycloalkylC₁-C₄alkyl, an optionally substituted C₆-C₁₀aryl, haloC₁-C₄alkyl, an optionally substituted C₆-C₁₀arylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
R² is C₃-C₇alkyl, C₂-C₆alkenyl, C₂-C₆alkynyl, C₁-C₇alkoxy, hydroxy, hydroxyC₁-C₄alkyl, haloC₁-C₄alkyl, C₁-C₆alkoxyC₁-C₄alkyl, an optionally substituted C₃-C₇cycloalkyl, an optionally substituted C₆-C₁₀aryl, an optionally substituted C₂-C₁₀heterocyclyl, or and optionally substituted C₁-C₁₀heteroaryl, provided that V—R² is not quinolin-4-ylmethyl when R¹ is an alkyl;
R³ is hydrogen, C₁-C₆alkyl, C₂-C₆alkenyl, C₂-C₆alkynyl, C₁-C₆alkoxy, hydroxyC₁-C₄alkyl, C₁-C₆alkoxyC₁-C₄alkyl, C₃-C₇cycloalkyl, C₃-C₇cycloalkylC₁-C₄alkyl, C₂-C₁₀heterocyclyl, C₆-C₁₀aryl, C₆-C₁₀arylC₁-C₄alkyl, C₁-C₁₀heteroaryl, halo, haloC₁-C₄alkyl, trifluoromethoxy, cyano, hydroxy, or —N(R⁸)₂;
R⁵ and R^{5a} are independently selected from hydrogen, C₁-C₆alkyl, haloC₁-C₄alkyl, hydroxy, hydroxyC₁-C₄alkyl, C₁-C₆alkoxy, C₃-C₇cycloalkylC₁-C₄alkyl and C₆-C₁₀arylC₁-C₄alkyl;
or R⁵ and R^{5a} are together to form an oxo (═O) group, or to form a C₃-C₇cycloalkyl;
R⁶, for each occurrence, is independently selected from C₁-C₆alkyl, C₆-C₁₀aryl, C₃-C₇cycloalkyl, C₁-C₁₀heteroaryl, C₂-C₁₀heterocyclyl, hydroxyC₁-C₄alkyl, haloC₁-C₄alkyl, C₁-C₆alkoxy, C₃-C₇cycloalkylC₁-C₄alkyl, C₆-C₁₀arylC₁-C₄alkyl-N(R⁸)C(O)R¹², —C(O)N(R⁸)R¹², —OC(O)N(R⁸)R¹², —N(R⁸)C(O)OR¹², —N(R⁸)C(O)N(R⁸)R¹², —OR¹², —SR¹², —N(R⁸)R¹², —S(O)ₜR¹², —N(R⁸)S(O)₂R¹², —S(O)₂N(R⁸)R¹², —OS(O)₂N(R⁸)R¹², —C(O)R¹², —OC(O)R¹², —N(R⁸)C(═N(R^{8a}))N(R⁸)R¹², —N(R⁸)C(═S)N(R⁸)R¹², —N(R⁸)((R^{8a})N═)CR¹², and —C(═N(R^{8a}))N(R⁸)R¹²;
or R⁵ and R⁶ on adjacent carbons together to form a C₃-C₇cycloalkyl or C₆-C₁₀aryl;
R⁷ is hydrogen, C₁-C₇alkyl, haloC₁-C₄alkyl, C₆-C₁₀aryl, C₃-C₇cycloalkyl, C₁-C₁₀heteroaryl, C₂-C₁₀heterocyclyl, hydroxyC₁-C₄alkyl, C₃-C₇cycloalkylC₁-C₄-C₄alkyl or aralkyl;
R⁸, for each occurrence, is independently selected from hydrogen, C₁-C₇alkyl, hydroxyC₁-C₄alkyl, C₃-C₇cycloalkyl, C₃-C₇cycloalkylC₁-C₄alkyl, C₆-C₁₀aryl, C₁-C₁₀heteroaryl, C₂-C₁₀heterocyclyl and aralkyl; and
R^{8a}, for each occurrence, is independently selected from hydrogen, C₁-C₇alkyl, C₃-C₇cycloalkyl, C₃-C₇cycloalkylC₁-C₄alkyl, and cyano;
R¹², for each occurrence, is independently selected from hydrogen, C₃-C₇alkyl, C₂-C₆alkenyl, C₂-C₆alkynyl, C₁-C₇alkoxy, hydroxy, hydroxyC₁-C₄alkyl, C₁-C₆alkoxyC₁-C₄alkyl, C₃-C₇cycloalkyl, C₃-C₇cycloalkylC₁-C₄alkyl, C₆-C₁₀aryl, haloC₁-C₄alkyl, aralkyl, aralkyloxy, C₂-C₁₀heterocyclyl, C₂-C₁₀heterocyclylC₁-C₄alkyl, C₁-C₁₀heteroaryl, and C₁-C₁₀heteroarylC₁-C₄alkyl; or a pharmaceutically acceptable salt thereof.

2. The compound, or a pharmaceutically acceptable salt thereof, according to claim 1, wherein Q is

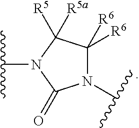

3. The compound, or a pharmaceutically acceptable salt thereof, according to claim 2, wherein Q is

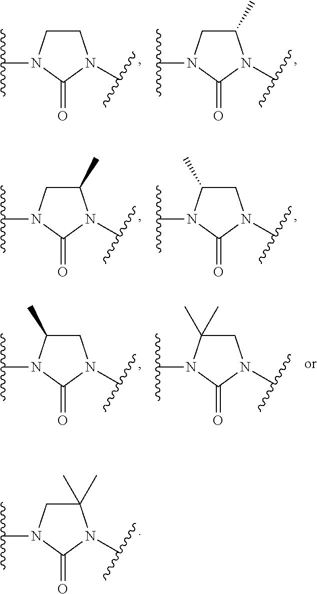

4. The compound, or a pharmaceutically acceptable salt thereof, according to claim 1, wherein Q is

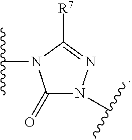

5. The compound, or a pharmaceutically acceptable salt thereof, according to claim 4, wherein Q is

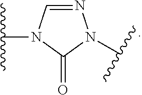 [SMILES: *n1cnn(*)c1=O]

6. The compound or a pharmaceutically acceptable salt thereof, according to claim 1, wherein W is —N(R⁸)C(O)—, and R¹ is hydrogen, C₁-C₇alkyl, an optionally substituted C₆-C₁₀aryl, an optionally substituted C₆-C₁₀arylC₁-C₄alkyl or an optionally substituted C₁-C₁₀heteroarylC₁-C₄alkyl.

7. The compound, or a pharmaceutically acceptable salt thereof, according to claim 6, wherein the aryl or heteroaryl group of C₆-C₁₀aryl, C₆-C₁₀arylC₁-C₄alkyl, C₁-C₁₀heteroaryl or C₁-C₁₀heteroarylC₁-C₄alkyl are optionally substituted with one or more substituents independently selected from the group consisting of C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄a141, C₂-C₆heterocyclyl, C₂-C₆heterocyclylC₁-C₄alkyl, C₁-C₁₀heteroaryl or C₁-C₁₀heteroarylalkyl; and where each t is 1 to 2.

8. The compound, or a pharmaceutically acceptable salt thereof, according to claim 1, wherein W is a direct bond and R¹ is an optionally substituted C₆-C₁₀aryl or an optionally substituted C₁-C₁₀heteroaryl.

9. The compound, or a pharmaceutically acceptable salt thereof, according to claim 8, wherein the aryl or heteroaryl group of R¹ are optionally substituted with one or more substituents independently selected from the group consisting of C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₂-C₆heterocyclyl, C₂-C₆heterocyclylC₁-C₄-C₄, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₇heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

10. The compound according to claim 1, wherein R² is hydroxy, an optionally substituted C₃-C₇cycloalkyl, haloC₁-C₄alkyl, an optionally substituted C₆-C₁₀aryl, an optionally substituted C₆-C₁₀arylC₁-C₄alkyloxy or an optionally substituted C₁-C₁₀heteroaryl.

11. The compound, or a pharmaceutically acceptable salt thereof, according to claim 10, wherein the aryl group of the arylalkyloxy, the cycloalkyl, aryl and heteroaryl are optionally substituted with one or more substituents independently selected from the group consisting of of C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁶—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁶ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆ heterocyclylC₁-C₄alkyl, C₁-C₁₀heteroaryl or C₁-C₁₀heteroarylalkyl; and where each t is 1 to 2.

12. The compound, or a pharmaceutically acceptable salt thereof, according to claim 1, wherein V—R² is selected from the group consisting of:

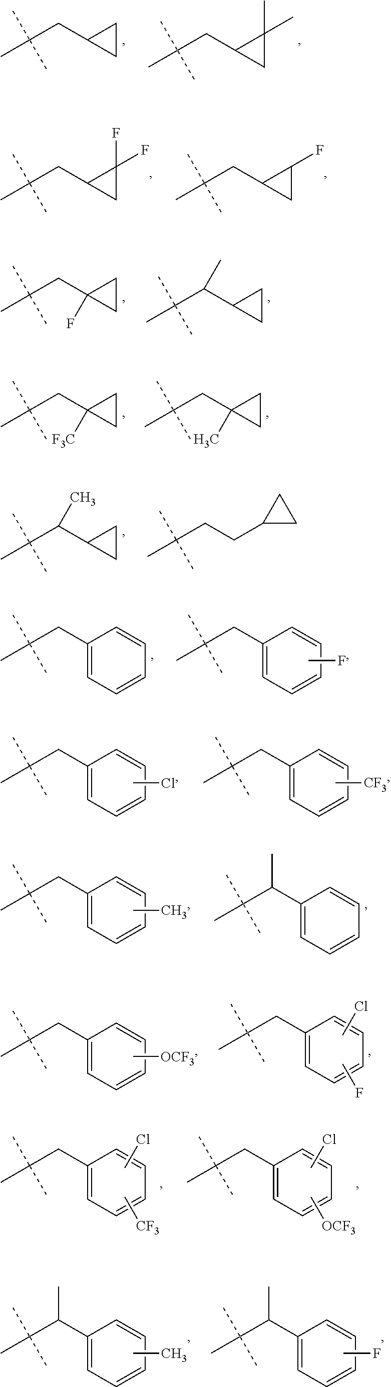

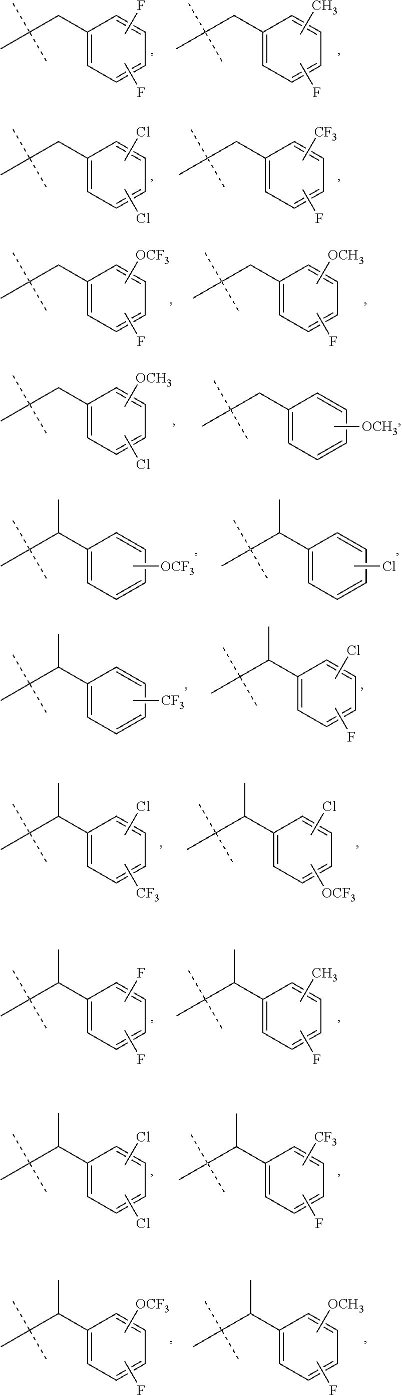

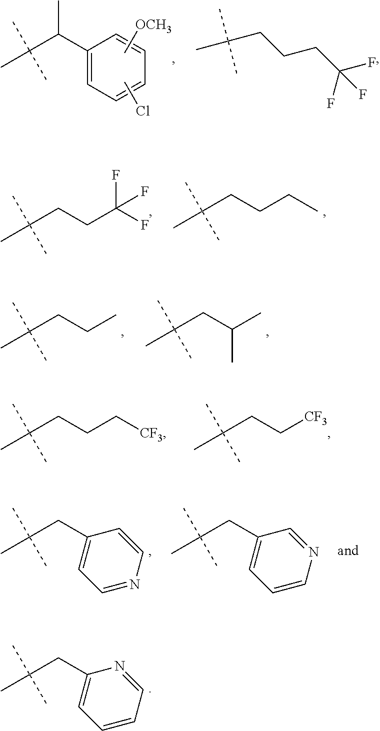

13. The compound, or a pharmaceutically acceptable salt thereof, according to claim 1, wherein
—V—R² is

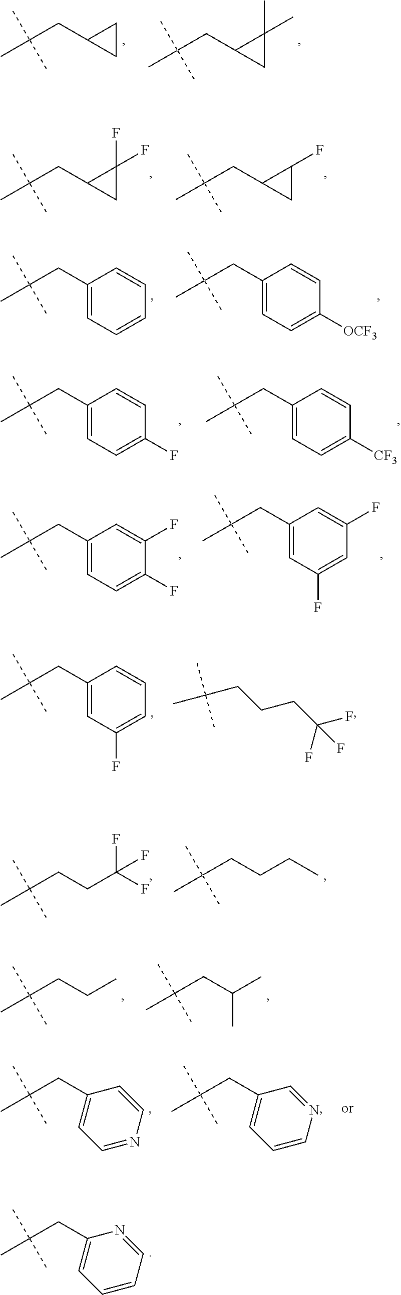

14. The compound, or a pharmaceutically acceptable salt thereof, according to claim 1, wherein; 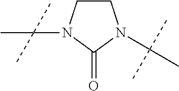
Q is
W is —N(R⁸)C(O)—;
V is a C₁-C₆alkylene;
R¹ is hydrogen, C₁-C₇alkyl, an optionally substituted C₆-C₁₀aryl, an optionally substituted C₆-C₁₀arylC₁-C₄alkyl, an optionally substituted C₃-C₇cycloalkyl, an optionally substituted C₃-C₇cycloalkylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
R² is hydroxy, C₃-C₇alkyl, haloC₁-C₄alkyl, an optionally substituted C₃-C₇cycloalkyl, an optionally substituted C₆-C₁₀aryl, or an optionally substituted C₁-C₁₀heteroaryl;
R³ is hydrogen; and
R⁸ is hydrogen or C₁-C₄alkyl.

15. The compound, or a pharmaceutically acceptable salt thereof, according to claim 1, wherein
Q is 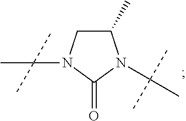
W is —N(R⁸)C(O)—;
V is a C₁-C₆alkylene;
R¹ is hydrogen, an optionally substituted aralkyl, or an optionally substituted C₁-C₁₀heteroarylC₁-C₄alkyl;
R² is C₃-C₇ alkyl, haloC₁-C₄alkyl, an optionally substituted C₃-C₇cycloalkyl, an optionally substituted C₆-C₁₀aryl, or an optionally substituted C₁-C₁₀heteroaryl;
R³ is hydrogen; and
R⁸ is hydrogen.

16. The compound, or a pharmaceutically acceptable salt thereof, according to claim 1, wherein
Q is 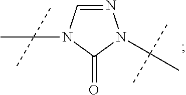
W is —N(R⁸)C(O)— or a direct bond;
V is a C₁-C₆alkylene;
R¹ is hydrogen, C₁-C₄alkyl, an optionally substituted C₆-C₁₀aryl, an optionally substituted C₆-C₁₀arylC₁-C₄alkyl, an optionally substituted C₃-C₇cycloalkyl, an optionally substituted C₃-C₇cycloalkylC₁-C₄alkyl, an optionally substituted C₂-C₁₀heterocyclyl, an optionally substituted C₂-C₁₀heterocyclylC₁-C₄alkyl, an optionally substituted C₁-C₁₀heteroaryl, or an optionally substituted C₁-C₁₀heteroarylC₁-C₄alkyl;
R² is a C₃-C₇ alkyl, haloC₁-C₄alkyl, an optionally substituted C₃-C₇cycloalkyl, an optionally substituted C₆-C₁₀aryl, or an optionally substituted C₁-C₁₀heteroaryl;
R³ is hydrogen; and
R⁸ is hydrogen or C₁-C₄alkyl.

17. The compound, or a pharmaceutically acceptable salt thereof, according to claim 1, wherein each aryl, cycloalkyl, heterocyclyl, or heteroaryl portion of an R¹ or R² group is independently optionally substituted with one or more substituents selected from C₁-C₆alkyl, C₂-C₆alkenyl, C₂-C₆alkynyl, halo, C₁-C₆haloalkyl, cyano, nitro, C₆-C₁₀aryl, C₆-C₁₀aryl C₁-C₄alkyl, C₃-C₇cycloalkyl, C₃-C₇cycloalkylC₁-C₄alkyl, C₂-C₆heterocyclyl, C₂-C₆heterocyclylC₁-C₄alkyl, C₁-C₁₀heteroaryl, C₁-C₁₀heteroarylC₁-C₆alkyl, —R¹⁵—OR¹⁴, —R¹⁵—OC(O)—R¹⁴, —R¹⁵—N(R¹⁴)₂, —R¹⁵—C(O)R¹⁴, —R¹⁵—C(O)OR¹⁴, —R¹⁵—C(O)N(R¹⁴)₂, —R¹⁵—N(R¹⁴)C(O)OR¹⁶, —R¹⁵—N(R¹⁴)C(O)R¹⁶, —R¹⁵—N(R¹⁴)(S(O)ₜR¹⁶), —R¹⁵—SR¹⁶, —R¹⁵—S(O)ₜR¹⁶, and —R¹⁵—S(O)ₜN(R¹⁴)₂, where each R¹⁴ is independently hydrogen, C₁-C₆alkyl, C₁-C₆haloalkyl, C₃-C₇cycloalkyl, C₃-C₇cycloalkylalkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl, or C₁-C₁₀heteroarylC₁-C₄alkyl; each R¹⁵ is independently a direct bond or a straight or branched C₁-C₆alkylene or C₁-C₆alkenylene chain; and each R¹⁶ is C₁-C₆alkyl, C₁-C₆haloalkyl, C₃-C₇cycloalkyl, C₃-C₇cycloalkylC₁-C₄alkyl, C₆-C₁₀aryl, C₆-C₁₀arylC₁-C₄alkyl, C₂-C₆heterocyclyl, C₂-C₆heterocyclylC₁-C₄alkyl, C₁-C₁₀heteroaryl or C₁-C₁₀heteroarylalkyl; and where each t is 1 to 2.

18. The compound, or a pharmaceutically acceptable salt thereof, according to claim 1, wherein —V—R² is selected from the group consisting of:

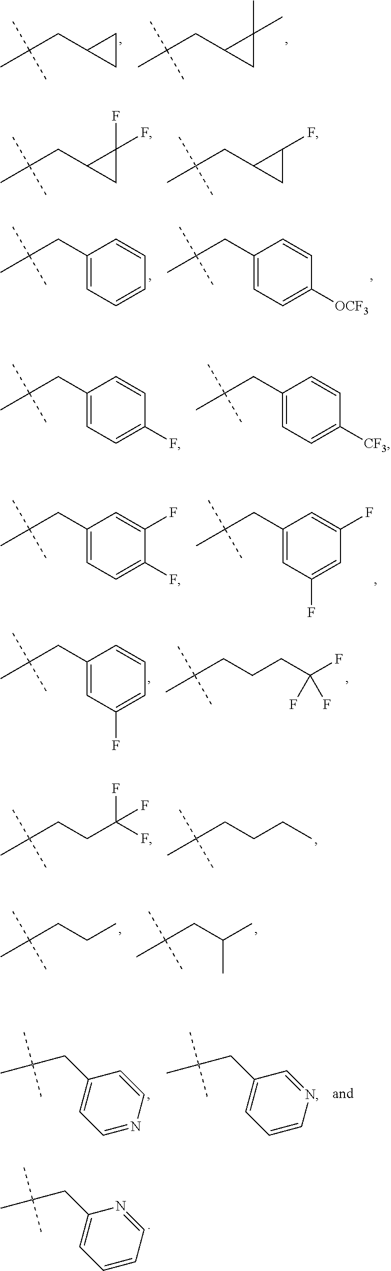

19. The compound, or a pharmaceutically acceptable salt thereof, according to claim 6, wherein
W is —N(R⁸)C(O)—, and
R¹ is hydrogen, C₁-C₄alkyl,

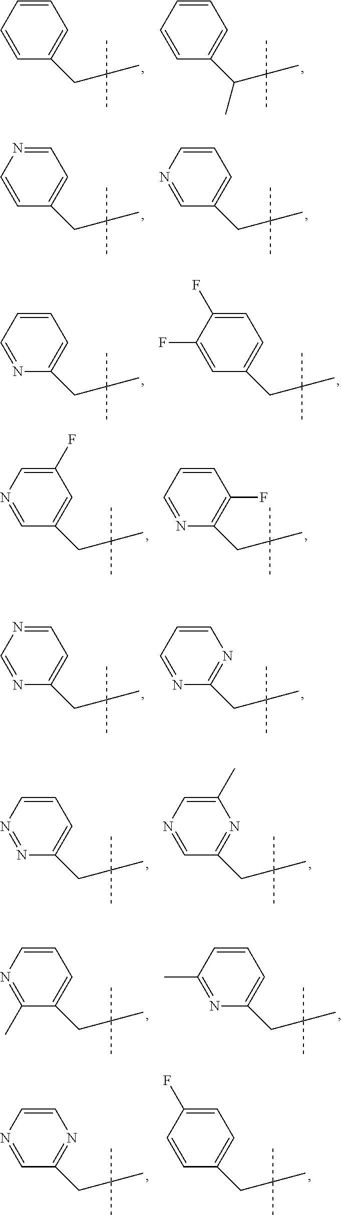

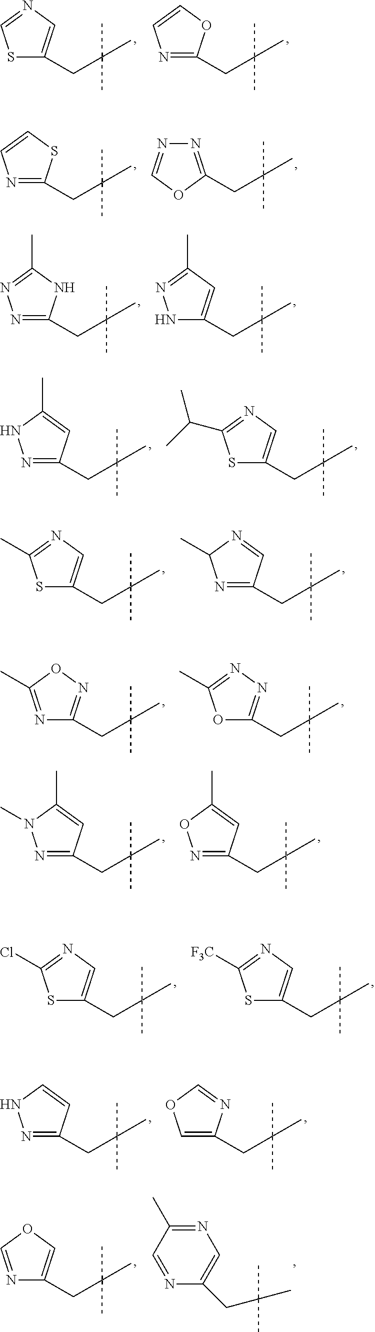

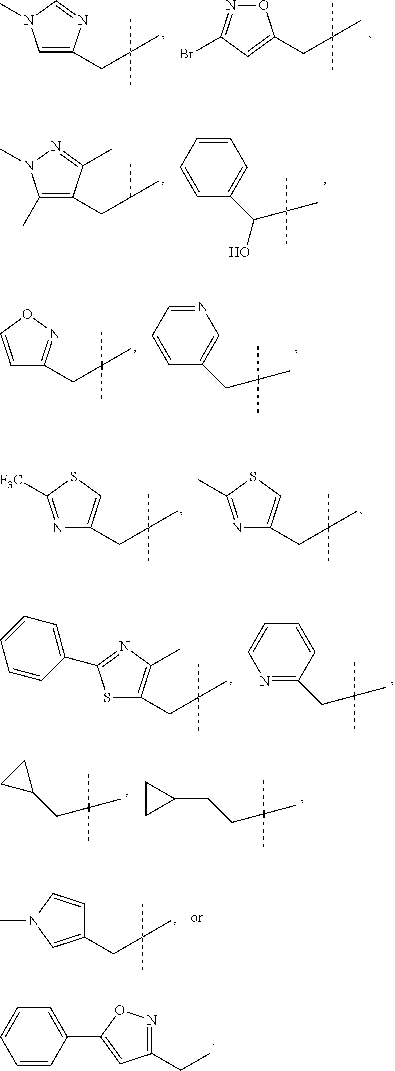

20. The compound, or a pharmaceutically acceptable salt thereof, according to claim 8 wherein
W is a direct bond and
R₁ is

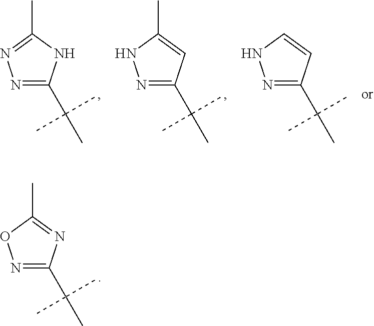

21. The compound, or a pharmaceutically acceptable salt thereof, according to claim 1, wherein —V—R² is selected from the group consisting of:

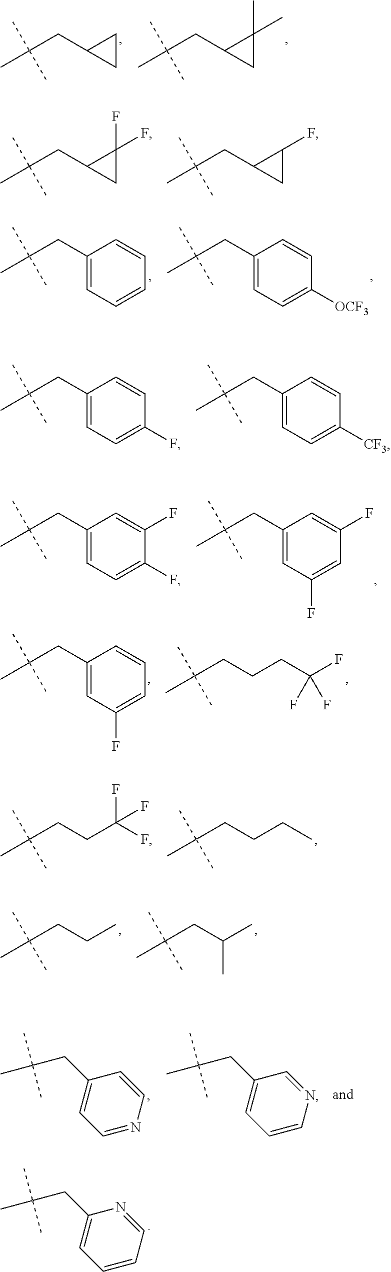

22. The compound according to claim 1, wherein the compound is
N-(3,4-Difluorobenzyl)-3-(1-(4-fluorobenzyl)-5-oxo-1H-1,2,4-triazol-4(5H)-yl)-1H-pyrazole-5-carboxamide,
3-(1-(4-Fluorobenzyl)-5-oxo-1H-1,2,4-triazol-4(5H)-yl)-N-(pyridin-3-ylmethyl)-1H-pyrazole-5-carboxamide,
N-Benzyl-3-(1-(4-fluorobenzyl)-5-oxo-1H-1,2,4-triazol-4(5H)-yl)-1H-pyrazole-5-carboxamide,
3-(1-(4-Fluorobenzyl)-5-oxo-1H-1,2,4-triazol-4(5H)-yl)-N-methyl-1H-pyrazole-5-carboxamide,
3-(3-(4-Fluorobenzyl)-2-oxoimidazolidin-1-yl)-N-(pyridin-2-ylmethyl)-1H-pyrazole-5-carboxamide,
3-(3-(4-Fluorobenzyl)-2-oxoimidazolidin-1-yl)-N-((1-methyl-1H-pyrazol-4-yl)methyl)-1H-pyrazole-5-carboxamide,
3-(1-(4-Fluorobenzyl)-5-oxo-1H-1,2,4-triazol-4(5H)-yl)-N-(oxazol-4-ylmethyl)-1H-pyrazole-5-carboxamide,
3-(1-(4-Fluorobenzyl)-5-oxo-1H-1,2,4-triazol-4(5H)-yl)-N-((3-methyl-1H-pyrazol-5-yl)methyl)-1H-pyrazole-5-carboxamide,
3-(1-(4-Fluorobenzyl)-5-oxo-1H-1,2,4-triazol-4(5H)-yl)-N-(pyridin-4-ylmethyl)-1H-pyrazole-5-carboxamide,
3-(1-(4-Fluorobenzyl)-5-oxo-1H-1-1,2,4-triazol-4(5H)-yl)-N-(pyridin-2-ylmethyl)-1H-pyrazole-5-carboxamide,
3-(3-(4-Fluorobenzyl)-2-oxoimidazolidin-1-yl)-N-((5-methyl-1H-pyrazol-3-yl)methyl)-1H-pyrazole-5-carboxamide,
3-(3-(4-Fluorobenzyl)-2-oxoimidazolidin-1-yl)-N-(thiazol-2-ylmethyl)-1H-pyrazole-5-carboxamide,
N-Benzyl-3-(3-(4-fluorobenzyl)-2-oxoimidazolidin-1-yl)-1H-pyrazole-5-carboxamide,
3-(3-(4-Fluorobenzyl)-2-oxoimidazolidin-1-yl)-N-((5-methylisoxazol-3-yl)methyl)-1H-pyrazole-5-carboxamide,
3-(3-(4-Fluorobenzyl)-2-oxoimidazolidin-1-yl)-1H-pyrazole-5-carboxamide,
3-(3-(4-Fluorobenzyl)-2-oxoimidazolidin-1-yl)-N-(pyridin-3-ylmethyl)-1H-pyrazole-5-carboxamide,
3-(1-(4-Fluorobenzyl)-5-oxo-1H-1,2,4-triazol-4(5H)-yl)-1H-pyrazole-5-carboxamide, and
3-(1-(4-Fluorobenzyl)-5-oxo-1H-1,2,4-triazol-4(5H)-yl)-N-((5-methylisoxazol-3-yl)m ethyl)-1H-pyrazole-5-carboxamide;
or a pharmaceutically acceptable salt thereof.

23. A pharmaceutical composition, comprising:
the compound of Formula (I), or a pharmaceutically acceptable salt thereof, according to claim 1 and
a pharmaceutically acceptable excipient or carrier.

24. A method of inhibiting human stearoyl-CoA desaturase (hSCD) activity comprising:
contacting a source of hSCD with the compound of Formulae (I) or
pharmaceutically acceptable salt thereof, according to claim 1.

25. A method of treating a disease or condition mediated by stearoyl-CoA desaturase (SCD) in a mammal, comprising:
administering to the mammal in need thereof a therapeutically effective amount of the compound of Formula (I), or a pharmaceutically acceptable salt thereof, according to claim 1.

26. The method according to claim 25, wherein the disease or condition is metabolic syndrome, Syndrome X, diabetes, insulin resistance, hyperinsulinanemia, reperfusion injury, angiplastic restenosis, thrombosis, decreased glucose tolerance, non-insulin-dependent diabetes mellitus, Type II diabetes, Type I diabetes, diabetic complications, body weight disorders, weight loss, body mass index or leptin related diseases.

27. The method according to claim 26, wherein the metabolic syndrome is dyslipidemia, obesity, insulin resistance, hypertension, microalbuminemia, hyperuricaemia, or hypercoagulability.

28. The method according to claim 26, wherein the bodyweight disorder is obesity, overweight, cachexia or anorexia.

29. The method according to claim 25, where the disease or condition is a skin disorder.

30. The method according to claim 29, wherein the skin disorder is eczema, acne, psoriasis, or keloid scar formation or prevention.

31. A pharmaceutical composition comprising a therapeutically effective amount of a compound, or a pharmaceutically acceptable sett thereof, of claim 1 in combination with a therapeutically effective amount of insulin, an insulin derivative or mimetic; an insulin secretagogue; an insulinotropic sulfonylurea receptor ligand; a PPAR ligand; an insulin sensitizer; biguanide; an alpha-glucosidase inhibitors; GLP-1, a GLP-1 analog or mimetic; a DPPIV inhibitor; a HMG-CoA reductase inhibitor; a squalene synthase inhibitor; an FXR or LXR ligand; cholestyramine; a fibrate; nicotinic acid; or aspirin.

32-40. (canceled)
